(19) United States
(12) Patent Application Publication
(10) Pub. No.: US 2022/0001026 A1
(43) Pub. Date: Jan. 6, 2022

(54) USE OF MRNA ENCODING OX40L TO TREAT CANCER IN HUMAN PATIENTS

(71) Applicant: ModernaTX, Inc., Cambridge, MA (US)

(72) Inventors: Robert MEEHAN, Cambridge, MA (US); Sima ZACHAREK, Cambridge, MA (US); Kristen HOPSON, Arlington, MA (US); Joshua P. FREDERICK, Charlestown, MA (US)

(21) Appl. No.: 17/290,571

(22) PCT Filed: Nov. 8, 2019
(86) PCT No.: PCT/US2019/060381

§ 371 Date: Apr. 30, 2021

Related U.S. Application Data
(60) Provisional application No. 62/883,522, filed on Aug. 6, 2019.
(60) Provisional application No. 62/757,671, filed on Nov. 8, 2018.

(51) Int. Cl. A61K 48/00; A61K 45/06; A61P 35/00; A61K 9/50

(52) U.S. Cl. CPC A61K 48/005; A61K 9/5015; A61P 35/00; A61K 45/06

(57) ABSTRACT

The disclosure features methods for treating ovarian cancer, as well as other cancers such as solid tumors, lymphomas and epithelial origin cancers, by administering mRNA encoding an OX40L polypeptide. The disclosure also features compositions for use in the methods. The disclosure also features combination therapies, such as use of mRNA encoding an OX40L polypeptide in combination with a checkpoint inhibitor, such as an anti-PD-L1 antibody.

### CROSS-REFERENCE TO RELATED APPLICATIONS

This application claims the benefit of U.S. Provisional Patent Application Ser. No. 62/757,671 filed on Nov. 8, 2018, and U.S. Provisional Patent Application Ser. No. 62/883,522 filed on Aug. 6, 2019, the contents of each of which are herein incorporated by reference in their entireties.

### BACKGROUND

Cancer is a disease characterized by uncontrolled cell division and growth within the body. In the United States, roughly a third of all women and half of all men will experience cancer in their lifetime. Cancer cells utilize a number of mechanisms to evade the immune system, which results in persistence of tumor cells. Much research has focused on methods of stimulating the immune system to allow it to recognize and attack tumor cells. One area of intense research is the use of immune checkpoint blockade (CTLA-4, PD-1, and PD-L1) to turn on immune responses to tumor cells. The tumor necrosis factor receptor superfamily also contains molecules which may be useful as immunomodulators. For example, several anti-OX40 antibodies have been tested in the clinic for their ability to treat cancer. However, it is often difficult to reconcile in vitro and in vivo data in this area of research. Further data from human patients is required to determine what immunomodulatory therapies work best for what types of cancers and what therapeutic modalities (protein therapy, gene therapy, mRNA therapy) are most effective.

### SUMMARY OF DISCLOSURE

The present disclosure is based, in part, on the discovery that messenger RNA (mRNA) encoding human OX40 ligand (OX40L) administered by intratumoral injection to human ovarian cancer patients resulted in a significant reduction in tumor size or complete resolution of the tumor at the site of injection. As demonstrated herein, mRNA encoding human OX40L induced significant human OX40L expression by tumor cells and/or immune cells following intratumoral administration to human ovarian cancer patients. Significantly, a local regional abscopal effect was observed for proximal, uninjected tumors within the vicinity of the injection site. In addition, in many patients intratumoral injection of LNPs comprising mRNA encoding OX40L was found to promote a pro-inflammatory response post-treatment in multiple cases, including demonstration of interferon Type I (IFN-I) responses. Without being bound by theory, it is believed that the induction of OX40L expression by tumor cells and/or cells presenting tumor antigens following administration of an mRNA encoding human OX40L induces a specific cell-mediated immune response with systemic anti-tumor effects, resulting in a reduction in tumor size of both treated and untreated tumors in human ovarian cancer patients.

Accordingly, in some aspects the disclosure provides a method for treating ovarian cancer, or other cancers such as solid tumors, lymphomas or epithelial origin cancers (e.g., an epithelial cancer of ovary, fallopian tube or peritoneum), in a human patient by inducing or enhancing an anti-tumor immune response, comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating ovarian cancer, or other cancers such as solid tumors, lymphomas or epithelial origin cancers (e.g., an epithelial cancer of ovary, fallopian tube or peritoneum), in the patient by inducing or enhancing an anti-tumor immune response. In one embodiment, the treatment is given in combination with a checkpoint inhibitor.

In some aspects, the patient is administered a dose of mRNA selected from 1.0-8.0 mg, 1.0-6.0 mg, 1.0-4.0 mg, and 1.0-2.0 mg of mRNA. In some aspects, the patient is administered a dose of mRNA from 1.0-8.0 mg. In some aspects, the patient is administered a dose of mRNA from 1.0-6.0 mg. In some aspects, the patient is administered a dose of mRNA from 1.0-4.0 mg. In some aspects, the patient is administered a dose of mRNA from 1.0-2.0 mg.

In any of the foregoing or related aspects, the mRNA is administered in a dosing regimen selected from 7 to 28 days, 7 to 21 days, 7 to 14 days, 28 days, 21 days, 14 days and 7 days. In some aspects, the mRNA is administered in a dosing regimen of 28 days. In some aspects, the mRNA is administered in a dosing regimen of 21 days. In some aspects, the mRNA is administered in a dosing regimen of 14 days. In some aspects, the mRNA is administered in a dosing regimen of 7 days. In other aspects, the mRNA is administered every 2 weeks in a 28-day cycle.

In another aspects, the disclosure provides a method for treating ovarian cancer, or other cancers such as solid tumors, lymphomas or epithelial origin cancers (e.g., an epithelial cancer of ovary, fallopian tube or peritoneum), in a human patient by inducing or enhancing an anti-tumor immune response, comprising administering to the patient by intratumoral injection an effective amount of a pharmaceutical composition comprising: an LNP comprising an mRNA encoding a human OX40L polypeptide; and a pharmaceutically acceptable carrier, wherein the patient is administered a dose of 1.0-8.0 mg of mRNA in a dosing regimen from 7 to 21 days, thereby treating ovarian cancer, or other cancers such as solid tumors, lymphomas or epithelial origin cancers (e.g., an epithelial cancer of ovary, fallopian tube or peritoneum), in the patient by inducing or enhancing an anti-tumor immune response.

In some aspects, the patient is administered a dose of 1.0-6.0 mg mRNA. In other aspects, the patient is administered a dose of 1.0-4.0 mg mRNA. In yet other aspects, the patient is administered a dose of 1.0-2.0 mg mRNA. In some aspects, the patient is administered a dose of 1.0 mg mRNA. In other aspects, the patient is administered a dose of 2.0 mg mRNA. In yet other aspects, the patient is administered a dose of 4.0 mg mRNA. In some aspects, the patient is administered a dose of 8.0 mg mRNA.

In any of the foregoing or related aspects, the dose is administered every 14 days.

In any of the foregoing or related aspects, the mRNA is administered every 2 weeks in a 28-day cycle. In some aspects, the mRNA is administered every 2 weeks for 1-6 months. In other aspects, the mRNA is administered on day 1 and day 15 (±2 days) of a 28-day cycle until the tumor lesion resolves.

In any of the foregoing or related aspects, the mRNA is administered every 2 weeks in a 28-day cycle at a dose of 8.0 mg. In some aspects, the mRNA is administered at a dose of 8.0 mg on day 1 and day 15 (±2 days) of a 28-day cycle until the tumor lesion resolves. In some aspects, the mRNA is administered at a dose of 8.0 mg on day 1 and day 15 (±2 days) of cycle 1 of a 28-day cycle and on day 1 of subsequent 28 day cycles until the tumor lesion resolves.

In any of the foregoing or related aspects, treatment results in a reduction in tumor size or inhibition in tumor growth in the injected tumor in the patient. In some aspects, treatment results in a reduction in size or inhibition of growth in an uninjected tumor in the patient. In some aspects, the uninjected tumor is at a location proximal to the injected tumor in the patient. In other aspects, the uninjected tumor is at a location distal to the injected tumor in the patient. In some aspects, treatment results in a reduction in size or inhibition of growth of an uninjected tumor through an abscopal effect in the patient.

In any of the foregoing or related aspects, treatment results in increased expression of human OX40L polypeptide in the tumor. In some aspects, treatment results in increased expression of human OX40L polypeptide in immune cells in the tumor microenvironment.

In any of the foregoing or related aspects, the anti-tumor immune response in the patient comprises T cell activation, T cell proliferation, and/or T cell expansion. In some aspects, the T cells are CD4+ T cells. In other aspects, the T cells are CD8+ T cells. In yet other aspects, the T cells are CD4+ T cells and CD8+ T cells. In some aspects, the anti-tumor immune response results in a reduction in size or inhibition of growth of the injected tumor. In some aspects, the anti-tumor immune response results in a reduction in size or inhibition of growth of an uninjected tumor through an abscopal effect in the patient.

In any of the foregoing or related aspects, treatment results in a pro-inflammatory response post-treatment in multiple cases, including demonstration of IFN-I responses (e.g., INFα and/or INFβ induced genes).

In any of the foregoing or related aspects, the patient has a superficial tumor lesion amenable to injection. In another embodiment, in any of the foregoing or related aspects, the patient has a visceral tumor lesion. In one embodiment, imaging guidance (e.g., ultrasound, computer tomography and the like) is used to facilitate intratumoral injection of a visceral tumor lesion.

In any of the foregoing or related aspects, the mRNA is administered by a single injection. In some aspects, the mRNA is administered by multiple injections into one or more different sites within the same tumor lesion or divided across several tumor lesions. For example, in some embodiments the mRNA is split across several lesions when no single lesion is available that is large enough to receive the entire dose in the maximum injection volume per lesion size.

In any of the foregoing or related aspects, the pharmaceutically acceptable carrier is a solution suitable for intratumoral injection. In some aspects, the solution comprises a buffer.

In any of the foregoing or related aspects, the patient to be treated has not responded to at least one prior anti-cancer treatment or at least one prior anti-cancer treatment has become ineffective (e.g., no longer inhibits tumor progression effectively). In some aspects, the prior anti-cancer treatment is a chemotherapy treatment. In some aspects, the prior anti-cancer treatment is a radiotherapy treatment. In some aspects, the prior anti-cancer treatment is an immunotherapy treatment.

In any of the foregoing or related aspects, the human OX40L polypeptide comprises the amino acid sequence set forth in SEQ ID NO: 1.

In any of the foregoing or related aspects, the mRNA comprises an open reading frame comprising a nucleotide sequence at least 90% identical to the nucleotide sequence set forth in SEQ ID NO: 4. In any of the foregoing or related aspects, the mRNA comprises an open reading frame comprising a nucleotide sequence at least 91%, 92%, 93%, 94%, 95%, 96%, 97%, 98% or 99% identical to the nucleotide sequence set forth in SEQ ID NO: 4. In some aspects, the mRNA comprises an open reading frame comprising the nucleotide sequence set forth in SEQ ID NO: 4. In some aspects, the mRNA comprises a 3′ untranslated region (UTR) comprising at least one microRNA-122 (miR-122) binding site. In some aspects, the miR-122 binding site is a miR-122-3p binding site. In other aspects, the miR-122 binding site is a miR-122-5p binding site. In some aspects, the miR-122-5p binding site comprises the nucleotide sequence set forth in SEQ ID NO: 20. In some aspects, the 3′UTR comprising a miR-122 binding site comprises the nucleotide sequence set forth in SEQ ID NO: 17. In some aspects, the mRNA comprises a 5′ untranslated region (UTR) comprising the nucleotide sequence set forth in SEQ ID NO: 15. In some aspects, the mRNA comprises a 5′ cap. In some aspects, the mRNA comprises a poly-A tail of about 100 nucleotides in length.

In some aspects, the mRNA comprises (i) a 5′UTR comprising the nucleotide sequence set forth in SEQ ID NO: 15; (ii) an open reading frame comprising the nucleotide sequence set forth in SEQ ID NO: 4; and (iii) a 3′UTR comprising the nucleotide sequence set forth in SEQ ID NO: 17.

In some aspects, the mRNA comprises (i) a 5′UTR comprising a nucleotide sequence having at least 90% identity to the nucleotide sequence set forth in SEQ ID NO: 15; (ii) an open reading frame comprising a nucleotide sequence having at least 90% identity to the nucleotide sequence set forth in SEQ ID NO: 4; and (iii) a 3′UTR comprising a nucleotide sequence having at least 90% identity to the nucleotide sequence set forth in SEQ ID NO: 17.

In some aspects, the mRNA comprises (i) a 5′UTR comprising a nucleotide sequence having at least 91%, 92%, 93%, 94%, 95%, 96%, 97%, 98% or 99% identity to the nucleotide sequence set forth in SEQ ID NO: 15; (ii) an open reading frame comprising a nucleotide sequence having at least 91%, 92%, 93%, 94%, 95%, 96%, 97%, 98% or 99% identity to the nucleotide sequence set forth in SEQ ID NO: 4; and (iii) a 3′UTR comprising a nucleotide sequence having at least 91%, 92%, 93%, 94%, 95%, 96%, 97%, 98% or 99% identity to the nucleotide sequence set forth in SEQ ID NO: 17.

In any of the foregoing or related aspects, the mRNA comprises a nucleotide sequence at least 90% identical to the nucleotide sequence set forth in SEQ ID NO: 5. In any of the foregoing or related aspects, the mRNA comprises a nucleotide sequence at least 91%, 92%, 93%, 94%, 95%, 96%, 97%, 98% or 99% identical to the nucleotide sequence set forth in SEQ ID NO: 5. In some aspects, the mRNA comprises the nucleotide sequence set forth in SEQ ID NO: 5.

In some aspects the disclosure provides a method for treating ovarian cancer in a human patient by inducing or enhancing an anti-tumor immune response, comprising administering to the patient by intratumoral injection an effective amount of a pharmaceutical composition comprising: a lipid nanoparticle (LNP) comprising a messenger RNA (mRNA) encoding a human OX40L polypeptide comprising a nucleotide sequence selected from the group consisting of the nucleotide sequence set forth in SEQ ID NO: 5 and a nucleotide sequence having at least 90%, 91%, 92%, 93%, 94%, 95%, 96%, 97%, 98% or 99% identity to the nucleotide sequence set forth in SEQ ID NO: 5; and a pharmaceutically acceptable carrier, thereby treating ovarian cancer in the patient by inducing or enhancing an anti-tumor immune response.

In any of the foregoing or related aspects, the mRNA is chemically modified. In some aspects, the mRNA is fully modified with chemically-modified uridines. In some aspects, the chemically-modified uridines are N1-methylpseudouridines (m1ψ). In some aspects, the mRNA is fully modified with 5-methylcytosine or is fully modified with N1-methylpseudouridines (m1ψ) and 5-methylcytosine.

In some aspects the disclosure provides a method for treating ovarian cancer, or other cancers such as solid tumors, lymphomas or epithelial origin cancers (e.g., an epithelial cancer of ovary, fallopian tube or peritoneum), in a human patient by inducing or enhancing an anti-tumor immune response, comprising administering to the patient by intratumoral injection an effective amount of a pharmaceutical composition comprising: a lipid nanoparticle (LNP) comprising a messenger RNA (mRNA) encoding a human OX40L polypeptide comprising a nucleotide sequence selected from the group consisting of the nucleotide sequence set forth in SEQ ID NO: 5 and a nucleotide sequence having at least 90% identity to the nucleotide sequence set forth in SEQ ID NO: 5, wherein the mRNA is fully modified with N1-methylpseudouridines (m1ψ) and 5-methylcytosine; and a pharmaceutically acceptable carrier, thereby treating ovarian cancer, or other cancers such as solid tumors, lymphomas or epithelial origin cancers (e.g., an epithelial cancer of ovary, fallopian tube or peritoneum), in the patient by inducing or enhancing an anti-tumor immune response.

In any of the foregoing or related aspects, the LNP comprises a compound having the formula:

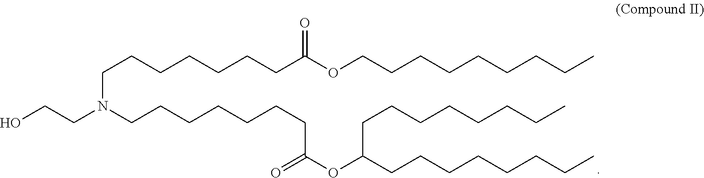 [SMILES: CCCCCCCCCOC(=O)CCCCCCCN(CCO)CCCCCCCC(=O)OC(CCCCCCCC)CCCCCCCC]

In some aspects, the LNP further comprising a phospholipid, a structural lipid, and a PEG lipid.

In any of the foregoing or related aspects, the LNP comprises a molar ratio of about 20-60% ionizable amino lipid, about 5-25% phospholipid, about 25-55% structural lipid, and about 0.5-1.5% PEG lipid. In some aspects, the LNP comprises a molar ratio of about 50% ionizable amino lipid, about 10% phospholipid, about 38.5% structural lipid, and about 1.5% PEG lipid. In other aspects, the LNP comprises a molar ratio of about 50% ionizable amino lipid, about 10% phospholipid, about 38.5% cholesterol, and about 1.5% PEG-DMG. In some aspects, the ionizable amino lipid comprises a compound having the formula:

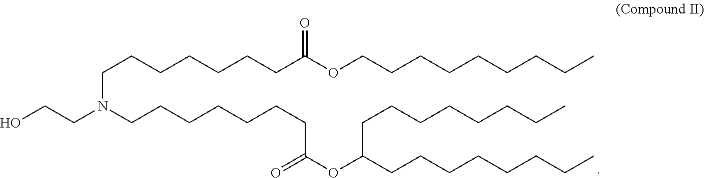 [SMILES: CCCCCCCCCOC(=O)CCCCCCCN(CCO)CCCCCCCC(=O)OC(CCCCCCCC)CCCCCCCC]

In any of the foregoing or related aspects, treatment further comprises administering an effective amount of a checkpoint inhibitor, e.g., PD-1 antagonist, a PD-L1 antagonist or a CTLA-4 antagonist. In some aspects, the PD-1 antagonist is an antibody or antigen binding portion thereof that specifically binds to PD-1. In some aspects, the PD-1 antagonist is selected from the group consisting of nivolumab, pembrolizumab, and pidilizumab. In some aspects, the PD-L1 antagonist is an antibody or antigen binding portion thereof that specifically binds to PD-L1. In some aspects, the PD-L1 antagonist is selected from the group consisting of durvalumab, avelumab, and atezolizumab. In some aspects, the PD-L1 antagonist is druvalumab. In some aspects, the CTLA-4 antagonist is an antibody or antigen binding portion thereof that specifically binds to CTLA-4. In some aspects, the CTLA-4 antagonist is selected from the group consisting of ipilimumab and tremelimumab.

In some aspects the disclosure provides a method for treating ovarian cancer, or other cancers such as solid tumors, lymphomas or epithelial origin cancers (e.g., an epithelial cancer of ovary, fallopian tube or peritoneum), in a human patient by inducing or enhancing an anti-tumor immune response, comprising administering to the patient (i) by intratumoral injection an effective amount of a pharmaceutical composition comprising: an LNP comprising an mRNA encoding a human OX40L polypeptide; and a pharmaceutically acceptable carrier; and (ii) by intravenous injection an effective amount of a PD-L1 antagonist; thereby treating ovarian cancer, or other cancers such as solid tumors, lymphomas or epithelial origin cancers (e.g., an epithelial cancer of ovary, fallopian tube or peritoneum), in the patient by inducing or enhancing an anti-tumor immune response.

In some aspects, the patient is administered a dose of 1.0-8.0 mg mRNA. In other aspects, the patient is administered a dose of 4.0-8.0 mg mRNA. In yet other aspects, the patient is administered a dose of 6.0-8.0 mg mRNA. In some aspects, the patient is administered a dose of 2.0 mg mRNA. In other aspects, the patient is administered a dose of 4.0 mg mRNA. In yet other aspects, the patient is administered a dose of 6.0 mg mRNA. In some aspects, the patient is administered a dose of 8.0 mg mRNA.

In any of the foregoing or related aspects, the mRNA dose is administered every 14 days.

In any of the foregoing or related aspects, the mRNA is administered every 2 weeks in a 28-day cycle. In some aspects, the mRNA is administered every 2 weeks for 1-6 months. In other aspects, the mRNA is administered on day 1 and day 15 (±2 days) of a 28-day cycle until the tumor lesion resolves.

In any of the foregoing or related aspects, the mRNA is administered at a dose of 1.0-8.0 mg in a dosing regimen from 7 to 21 days. In some aspects, the mRNA is administered at a dose of 8.0 mg in a dosing regimen of once every two weeks or once every four weeks.

In any of the foregoing or related aspects, the mRNA is administered every 2 weeks in a 28-day cycle at a dose of 8.0 mg. In some aspects, the mRNA is administered at a dose of 8.0 mg on day 1 and day 15 (±2 days) of a 28-day cycle until the tumor lesion resolves. In some aspects, the mRNA is administered at a dose of 8.0 mg on day 1 and day 15 (±2 days) of cycle 1 of a 28-day cycle and on day 1 of subsequent 28 day cycles until the tumor lesion resolves.

In any of the foregoing or related aspects, the PD-L1 antagonist is selected from the group consisting of durvalumab, avelumab, and atezolizumab. In some aspects, the PD-L1 antagonist is durvalumab. In some aspects, the PD-L1 antagonist (e.g., durvalumab) is administered at a dose of 1500 mg. In some aspects, the PD-L1 antagonist (e.g., durvalumab) is administered in a dosing regimen of once every four weeks.

In some aspects, the patient is treated with the OX40L mRNA and the PD-L1 antagonist concurrently (i.e., treatment with the OX40L mRNA and the PD-L1 antagonist begins on the same day, or overlaps for at least a portion of the treatment period). In some aspects, treatment of the patient with the OX40L mRNA is initiated prior to treatment with the PD-L1 antagonist. In some aspects, treatment of the patient with the PD-L1 antagonist is initiated prior to treatment with the OX40L mRNA.

In another aspect, the disclosure pertains to use of the mRNA of the disclosure, or an LNP comprising an mRNA of the disclosure as well as methods of treatment, wherein the use or method comprises treating ovarian cancer in a human patient by administering to the patient, optionally by intratumoral injection, an effective amount of a pharmaceutical composition comprising: a lipid nanoparticle (LNP) comprising a messenger RNA (mRNA) encoding a human OX40L polypeptide; and a pharmaceutically acceptable carrier, thereby treating ovarian cancer in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg, optionally 8.0 mg; and

(iv) the mRNA is administered in a dosing regimen selected from once every 7 to 28 days, once every 7 to 21 days, once every 7 to 14 days, once every 28 days, once every 21 days, once every 14 days and once every 7 days.

In another aspect, the disclosure pertains to use of the mRNA of the disclosure, or an LNP comprising an mRNA of the disclosure as well as methods of treatment, wherein the use or method comprises treating ovarian cancer in a human patient by administering to the patient, optionally by intratumoral injection, an effective amount of a pharmaceutical composition comprising: a lipid nanoparticle (LNP) comprising a messenger RNA (mRNA) encoding a human OX40L polypeptide; and a pharmaceutically acceptable carrier, thereby treating ovarian cancer in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg, optionally 8 mg;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In another aspect, the disclosure pertains to use of the mRNA of the disclosure, or an LNP comprising an mRNA of the disclosure as well as methods of treatment, wherein the use or method comprises treating a solid tumor in a human patient by administering to the patient, optionally by intratumoral injection, an effective amount of a pharmaceutical composition comprising: a lipid nanoparticle (LNP) comprising a messenger RNA (mRNA) encoding a human OX40L polypeptide; and a pharmaceutically acceptable carrier, thereby treating the solid tumor in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg, optionally 8 mg; and

(iv) the mRNA is administered in a dosing regimen selected from once every 7 to 28 days, once every 7 to 21 days, once every 7 to 14 days, once every 28 days, once every 21 days, once every 14 days and once every 7 days.

In another aspect, the disclosure pertains to use of the mRNA of the disclosure, or an LNP comprising an mRNA of the disclosure as well as methods of treatment, wherein the use or method comprises treating a solid tumor in a human patient by administering to the patient, optionally by intratumoral injection, an effective amount of a pharmaceutical composition comprising: a lipid nanoparticle (LNP) comprising a messenger RNA (mRNA) encoding a human OX40L polypeptide; and a pharmaceutically acceptable carrier, thereby treating a solid tumor in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg, optionally 8 mg;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In another aspect, the disclosure pertains to use of the mRNA of the disclosure, or an LNP comprising an mRNA of the disclosure as well as methods of treatment, wherein the use or method comprises treating a lymphoma in a human patient by administering to the patient an effective amount of a pharmaceutical composition comprising: a lipid nanoparticle (LNP) comprising a messenger RNA (mRNA) encoding a human OX40L polypeptide; and a pharmaceutically acceptable carrier, thereby treating the lymphoma in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg, optionally 8 mg, optionally by intratumoral injection; and

(iv) the mRNA is administered in a dosing regimen selected from once every 7 to 28 days, once every 7 to 21 days, once every 7 to 14 days, once every 28 days, once every 21 days, once every 14 days and once every 7 days.

In another aspect, the disclosure pertains to use of the mRNA of the disclosure, or an LNP comprising an mRNA of the disclosure as well as methods of treatment, wherein the use or method comprises treating a lymphoma in a human patient, by administering to the patient an effective amount of a pharmaceutical composition comprising: a lipid nanoparticle (LNP) comprising a messenger RNA (mRNA) encoding a human OX40L polypeptide; and a pharmaceutically acceptable carrier, thereby treating the lymphoma in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg, optionally 8 mg, optionally by intratumoral injection;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In another aspect, the disclosure pertains to use of the mRNA of the disclosure, or an LNP comprising an mRNA of the disclosure as well as methods of treatment, wherein the use or method comprises treating an epithelial origin cancer in a human patient by administering to the patient, optionally by intratumoral injection, an effective amount of a pharmaceutical composition comprising: a lipid nanoparticle (LNP) comprising a messenger RNA (mRNA) encoding a human OX40L polypeptide; and a pharmaceutically acceptable carrier, thereby treating the epithelial origin cancer in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg, optionally 8 mg; and

(iv) the mRNA is administered in a dosing regimen selected from once every 7 to 28 days, once every 7 to 21 days, once every 7 to 14 days, once every 28 days, once every 21 days, once every 14 days and once every 7 days.

In another aspect, the disclosure pertains to use of the mRNA of the disclosure, or an LNP comprising an mRNA of the disclosure as well as methods of treatment, wherein the use or method comprises treating an epithelial origin cancer in a human patient by administering to the patient, optionally by intratumoral injection, an effective amount of a pharmaceutical composition comprising: a lipid nanoparticle (LNP) comprising a messenger RNA (mRNA) encoding a human OX40L polypeptide; and a pharmaceutically acceptable carrier, thereby treating the epithelial origin cancer in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg, optionally 8 mg;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In one embodiment of any of the foregoing methods, the epithelial origin cancer is an epithelial cancer of the ovary. In one embodiment of any of the foregoing methods, the epithelial origin cancer is an epithelial cancer of the fallopian tube. In one embodiment of any of the foregoing methods, the epithelial origin cancer is an epithelial cancer of the peritoneum.

In any of the foregoing or related aspects of the methods, the mRNA comprises an open reading frame at least 91%, 92%, 93%, 94%, 95%, 96%, 97%, 98% or 99% identical to the nucleotide sequence set forth in SEQ ID NO: 4. In one embodiment, the mRNA comprises an open reading frame comprising the nucleotide sequence set forth in SEQ ID NO: 4.

In any of the foregoing or related aspects of the methods, the mRNA comprises a nucleotide sequence at least 91%, 92%, 93%, 94%, 95%, 96%, 97%, 98% or 99% identical to the nucleotide sequence set forth in SEQ ID NO: 5. In one embodiment, the mRNA comprises the nucleotide sequence set forth in SEQ ID NO: 5.

In any of the foregoing or related aspects of the methods, the mRNA is administered at a dose of 8.0 mg. In any of the foregoing or related aspects, the mRNA is administered on day 1 and day 15 (±2 days) of a 28-day cycle for multiple cycles until the tumor lesion resolves or is administered on day 1 and day 15 (±2 days) of a 28-day cycle for one cycle and on day 1 of a 28-day cycle for multiple subsequent cycles until the tumor lesion resolves.

In any of the foregoing or related aspects of the methods, the mRNA is administered in combination with an immune checkpoint inhibitor. In one embodiment, the immune checkpoint inhibitor is an antagonist of PD-1/PD-L1 interaction. In one embodiment, the immune checkpoint inhibitor is a PD-1 antagonist. In one embodiment, the PD-1 antagonist is selected from the group consisting of nivolumab, pembrolizumab, and pidilizumab. In one embodiment, the immune check point inhibitor is a PD-L1 antagonist. In one embodiment, the PD-L1 antagonist is selected from the group consisting of durvalumab, avelumab, and atezolizumab. In one embodiment, the PD-L1 antagonist is durvalumab. In one embodiment, durvalumab is administered at a dose of 1500 mg in a dosing regimen of once every four weeks. In other embodiments, any of the other immune checkpoint inhibitors described herein (e.g., PD-L2 antagonists, CTLA-4 antagonists) is administered in combination with the mRNA.

In other aspects, the disclosure provides a kit comprising a container comprising a pharmaceutical composition comprising: a lipid nanoparticle comprising an mRNA encoding a human OX40L polypeptide; and a pharmaceutically acceptable carrier, wherein the pharmaceutical composition comprises 2 mg/ml of the mRNA, and a package insert comprising instructions for administration of the mRNA by intratumoral injection to treat or delay progression of ovarian cancer, or other cancers such as solid tumors, lymphomas or epithelial origin cancers (e.g., an epithelial cancer of ovary, fallopian tube or peritoneum), in a human patient.

In yet other aspects, the disclosure provides a kit comprising a container comprising a pharmaceutical composition comprising: a lipid nanoparticle comprising an mRNA encoding a human OX40L polypeptide; and a pharmaceutically acceptable carrier, wherein the pharmaceutical composition comprises 2 mg/ml of the mRNA, and a package insert comprising instructions for administration of the mRNA by intratumoral injection and instruction for use in combination with a second composition comprising a PD-1 antagonist, a PD-L1 antagonist or a CTLA-4 antagonist, for use in treating or delaying progression of ovarian cancer, or other cancers such as solid tumors, lymphomas or epithelial origin cancers (e.g., an epithelial cancer of ovary, fallopian tube or peritoneum), in a human patient. In some aspects, the PD-1 antagonist is an antibody or antigen binding portion thereof that specifically binds to PD-1. In some aspects, the PD-1 antagonist is selected from the group consisting of nivolumab, pembrolizumab, and pidilizumab. In some aspects, the PD-L1 antagonist is an antibody or antigen binding portion thereof that specifically binds to PD-L1. In some aspects, the PD-L1 antagonist is selected from the group consisting of durvalumab, avelumab, and atezolizumab. In some aspects, the PD-L1 antagonist is durvalumab. In some aspects, the CTLA-4 antagonist is an antibody or antigen binding portion thereof that specifically binds to CTLA-4. In some aspects, the CTLA-4 antagonist is selected from the group consisting of ipilimumab and tremelimumab.

In some aspects, the instructions provide administration of the lipid nanoparticle in a dosing regimen selected from 7 to 28 days, 7 to 21 days, 7 to 14 days, 28 days, 21 days, 14 days and 7 days. In some aspects, the instructions provide administration of the lipid nanoparticle every 2 weeks in a 28-day cycle. In some aspects, the instructions provide administration of the lipid nanoparticle at an mRNA dose of 1.0-8.0 mg, e.g., at 2.0 mg, 4.0 mg, 6.0 mg or 8.0 mg. In some aspects, the instructions provide administration of the PD-L1 antagonist (e.g., durvalumab) at a dose of 1500 mg.

In any of the foregoing or related aspects, the human OX40L polypeptide comprises the amino acid sequence set forth in SEQ ID NO: 1.

In any of the foregoing or related aspects, the mRNA comprises an open reading frame comprising a nucleotide sequence at least 90% identical to the nucleotide sequence set forth in SEQ ID NO: 4. In some aspects, the mRNA comprises an open reading frame comprising the nucleotide sequence set forth in SEQ ID NO: 4. In some aspects, the mRNA comprises a 3′ untranslated region (UTR) comprising at least one microRNA-122 (miR-122) binding site. In some aspects, the miR-122 binding site is a miR-122-3p binding site. In other aspects, the miR-122 binding site is a miR-122-5p binding site. In some aspects, the miR-122-5p binding site comprises the nucleotide sequence set forth in SEQ ID NO: 20. In some aspects, the 3′UTR comprising a miR-122 binding site comprises the nucleotide sequence set forth in SEQ ID NO: 17. In some aspects, the mRNA comprises a 5′ untranslated region (UTR) comprising the nucleotide sequence set forth in SEQ ID NO: 15. In some aspects, the mRNA comprises a 5′ cap. In some aspects, the mRNA comprises a poly-A tail of about 100 nucleotides in length.

In any of the foregoing or related aspects, the mRNA comprises a nucleotide sequence at least 90% identical to the nucleotide sequence set forth in SEQ ID NO: 5. In some aspects, the mRNA comprises the nucleotide sequence set forth in SEQ ID NO: 5.

In any of the foregoing or related aspects, the mRNA is chemically modified. In some aspects, the mRNA is fully modified with chemically-modified uridines. In some aspects, the chemically-modified uridines are N1-methylpseudouridines (m1ψ). In some aspects, the mRNA is fully modified with 5-methylcytosine or is fully modified with N1-methylpseudouridines (m1ψ) and 5-methylcytosine.

In any of the foregoing or related aspects, the LNP comprises a compound having the formula:

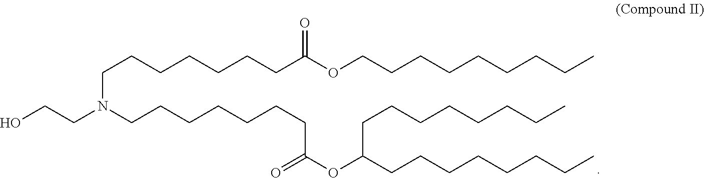 [SMILES: CCCCCCCCCOC(=O)CCCCCCCN(CCO)CCCCCCCC(=O)OC(CCCCCCCC)CCCCCCCC]

In some aspects, the LNP further comprising a phospholipid, a structural lipid, and a PEG lipid.

In any of the foregoing or related aspects, the LNP comprises a molar ratio of about 20-60% ionizable amino lipid, about 5-25% phospholipid, about 25-55% structural lipid, and about 0.5-1.5% PEG lipid. In some aspects, the LNP comprises a molar ratio of about 50% ionizable amino lipid, about 10% phospholipid, about 38.5% structural lipid, and about 1.5% PEG lipid. In other aspects, the LNP comprises a molar ratio of about 50% ionizable amino lipid, about 10% phospholipid, about 38.5% cholesterol, and about 1.5% PEG-DMG. In some aspects, the ionizable amino lipid comprises a compound having the formula:

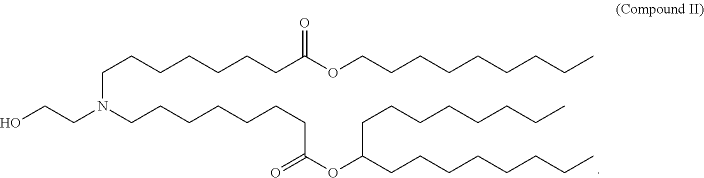 [SMILES: CCCCCCCCCOC(=O)CCCCCCCN(CCO)CCCCCCCC(=O)OC(CCCCCCCC)CCCCCCCC]

In another aspect, the disclosure pertains to a kit for the treatment of ovarian cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA, optionally by intratumoral injection, to treat or delay progression of ovarian cancer in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg, optionally 8.0 mg; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

In another aspect, the disclosure pertains to a kit for the treatment of ovarian cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of ovarian cancer in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In another aspect, the disclosure pertains to a kit for the treatment of a solid tumor in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the solid tumor in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

In another aspect, the disclosure pertains to a kit for the treatment of a solid tumor in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the solid tumor in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In another aspect, the disclosure pertains to a kit for the treatment of a lymphoma in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA to treat or delay progression of the lymphoma in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg, optionally by intratumoral injection; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

In another aspect, the disclosure pertains to a kit for the treatment of a lymphoma in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA to treat or delay progression of the lymphoma in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg, optionally by intratumoral injection;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In another aspect, the disclosure pertains to a kit for the treatment of an epithelial origin cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the epithelial origin cancer in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

In another aspect, the disclosure pertains to a kit for the treatment of an epithelial origin cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the epithelial origin cancer in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

In one embodiment of the foregoing kits, the epithelial origin cancer is an epithelial cancer of ovary, fallopian tube or peritoneum.

In any of the foregoing or related aspects of the kits, the mRNA can comprise an open reading frame at least 91%, 92%, 93%, 94%, 95%, 96%, 97%, 98% or 99% identical to the nucleotide sequence set forth in SEQ ID NO: 4. In one embodiment, the mRNA comprises an open reading frame comprising the nucleotide sequence set forth in SEQ ID NO: 4.

In any of the foregoing or related aspects of the kits, the mRNA can comprise a nucleotide sequence at least 91%, 92%, 93%, 94%, 95%, 96%, 97%, 98% or 99% identical to the nucleotide sequence set forth in SEQ ID NO: 5. In one embodiment, the mRNA comprises comprises the nucleotide sequence set forth in SEQ ID NO: 5.

In any of the foregoing or related aspects of the kits, the package insert can instruct administration of the mRNA at a dose of 8.0 mg. In any of the foregoing or related aspects, the package insert can instruct administration of the mRNA on day 1 and day 15 (±2 days) of a 28-day cycle for multiple cycles until the tumor lesion resolves or is administered on day 1 and day 15 (±2 days) of a 28-day cycle for one cycle and on day 1 of a 28-day cycle for multiple subsequent cycles until the tumor lesion resolves.

In any of the foregoing or related aspects of the kits, the package insert can instruct administration of the mRNA in combination with an immune checkpoint inhibitor. In one embodiment, the immune checkpoint inhibitor is an antagonist of PD-1/PD-L1 interaction. In one embodiment, the immune checkpoint inhibitor is a PD-1 antagonist. In one embodiment, the PD-1 antagonist is selected from the group consisting of nivolumab, pembrolizumab, and pidilizumab. In one embodiment, the immune check point inhibitor is a PD-L1 antagonist. In one embodiment, the PD-L1 antagonist is selected from the group consisting of durvalumab, avelumab, and atezolizumab. In one embodiment, the PD-L1 antagonist is durvalumab. In one embodiment, the package insert instructs administration of durvalumab at a dose of 1500 mg in a dosing regimen of once every four weeks. In other embodiments, any of the other immune checkpoint inhibitors described herein (e.g., PD-L2 antagonists, CTLA-4 antagonists) can be used in combination with the mRNA.

In some aspects, the disclosure provides an LNP comprising an mRNA encoding a human OX40L polypeptide, and an optional pharmaceutically acceptable carrier, for use in treating ovarian cancer in a human patient, wherein the LNP is administered by intratumoral injection. In some aspects, the disclosure provides a composition comprising an LNP comprising an mRNA encoding a human OX40L polypeptide, and a pharmaceutically acceptable carrier, for use in treating ovarian cancer in a human patient by inducing or enhancing an anti-tumor immune response, wherein the LNP is administered by intratumoral injection.

In some aspects, the disclosure provides an LNP comprising an mRNA encoding a human OX40L polypeptide, and an optional pharmaceutically acceptable carrier, for use in treating ovarian cancer in a human patient, wherein the LNP is administered by intratumoral injection at a dose of 1.0-8.0 mg of RNA in a dosing regimen from 7-21 days. In some aspects, the disclosure provides a composition comprising an LNP comprising an mRNA encoding a human OX40L polypeptide, and a pharmaceutically acceptable carrier, for use in treating ovarian cancer in a human patient by inducing or enhancing an anti-tumor immune response, wherein the LNP is administered by intratumoral injection at a dose of 1.0-8.0 mg of RNA in a dosing regimen from 7-21 days.

In some aspects, the disclosure provides an LNP comprising an mRNA encoding a human OX40L polypeptide, and an optional pharmaceutically acceptable carrier, for use in treating a solid tumor in a human patient, wherein the LNP is administered by intratumoral injection. In some aspects, the disclosure provides a composition comprising an LNP comprising an mRNA encoding a human OX40L polypeptide, and a pharmaceutically acceptable carrier, for use in treating a solid tumor in a human patient by inducing or enhancing an anti-tumor immune response, wherein the LNP is administered by intratumoral injection.

In some aspects, the disclosure provides an LNP comprising an mRNA encoding a human OX40L polypeptide, and an optional pharmaceutically acceptable carrier, for use in treating a solid tumor in a human patient, wherein the LNP is administered by intratumoral injection at a dose of 1.0-8.0 mg of RNA in a dosing regimen from 7-21 days. In some aspects, the disclosure provides a composition comprising an LNP comprising an mRNA encoding a human OX40L polypeptide, and a pharmaceutically acceptable carrier, for use in treating a solid tumor in a human patient by inducing or enhancing an anti-tumor immune response, wherein the LNP is administered by intratumoral injection at a dose of 1.0-8.0 mg of RNA in a dosing regimen from 7-21 days.

In some aspects, the disclosure provides an LNP comprising an mRNA encoding a human OX40L polypeptide, and an optional pharmaceutically acceptable carrier, for use in treating a lymphoma in a human patient, wherein the LNP is administered by intratumoral injection. In some aspects, the disclosure provides a composition comprising an LNP comprising an mRNA encoding a human OX40L polypeptide, and a pharmaceutically acceptable carrier, for use in treating a lymphoma in a human patient by inducing or enhancing an anti-tumor immune response, wherein the LNP is administered by intratumoral injection.

In some aspects, the disclosure provides an LNP comprising an mRNA encoding a human OX40L polypeptide, and an optional pharmaceutically acceptable carrier, for use in treating a lymphoma in a human patient, wherein the LNP is administered by intratumoral injection at a dose of 1.0-8.0 mg of RNA in a dosing regimen from 7-21 days. In some aspects, the disclosure provides a composition comprising an LNP comprising an mRNA encoding a human OX40L polypeptide, and a pharmaceutically acceptable carrier, for use in treating a lymphoma in a human patient by inducing or enhancing an anti-tumor immune response, wherein the LNP is administered by intratumoral injection at a dose of 1.0-8.0 mg of RNA in a dosing regimen from 7-21 days.

In some aspects, the disclosure provides an LNP comprising an mRNA encoding a human OX40L polypeptide, and an optional pharmaceutically acceptable carrier, for use in treating an epithelial origin cancer in a human patient, wherein the LNP is administered by intratumoral injection. In some aspects, the disclosure provides a composition comprising an LNP comprising an mRNA encoding a human OX40L polypeptide, and a pharmaceutically acceptable carrier, for use in treating an epithelial origin cancer in a human patient by inducing or enhancing an anti-tumor immune response, wherein the LNP is administered by intratumoral injection.

In some aspects, the disclosure provides an LNP comprising an mRNA encoding a human OX40L polypeptide, and an optional pharmaceutically acceptable carrier, for use in treating an epithelial origin cancer in a human patient, wherein the LNP is administered by intratumoral injection at a dose of 1.0-8.0 mg of RNA in a dosing regimen from 7-21 days. In some aspects, the disclosure provides a composition comprising an LNP comprising an mRNA encoding a human OX40L polypeptide, and a pharmaceutically acceptable carrier, for use in treating an epithelial origin cancer in a human patient by inducing or enhancing an anti-tumor immune response, wherein the LNP is administered by intratumoral injection at a dose of 1.0-8.0 mg of RNA in a dosing regimen from 7-21 days.

In some aspects, the disclosure provides a composition comprising an LNP comprising an mRNA encoding a human OX40L polypeptide and a pharmaceutically acceptable carrier for use in treating ovarian cancer in a human patient in a treatment regimen with a PD-L1 antagonist, wherein the composition is administered by intratumoral injection and wherein the PD-L1 antagonist is administered by intravenous injection.

In some aspects, the disclosure provides a composition comprising an LNP comprising an mRNA encoding a human OX40L polypeptide and a pharmaceutically acceptable carrier for use in treating a solid tumor in a human patient in a treatment regimen with a PD-L1 antagonist, wherein the composition is administered by intratumoral injection and wherein the PD-L1 antagonist is administered by intravenous injection.

In some aspects, the disclosure provides a composition comprising an LNP comprising an mRNA encoding a human OX40L polypeptide and a pharmaceutically acceptable carrier for use in treating a lymphoma in a human patient in a treatment regimen with a PD-L1 antagonist, wherein the composition is administered by intratumoral injection and wherein the PD-L1 antagonist is administered by intravenous injection.

In some aspects, the disclosure provides a composition comprising an LNP comprising an mRNA encoding a human OX40L polypeptide and a pharmaceutically acceptable carrier for use in treating an epithelial origin cancer in a human patient in a treatment regimen with a PD-L1 antagonist, wherein the composition is administered by intratumoral injection and wherein the PD-L1 antagonist is administered by intravenous injection.

### BRIEF DESCRIPTION OF FIGURES

FIG. 1 provides a schematic depicting the clinical study design showing the dosing and tumor biopsy schedule for each biopsy cohort, as indicated.

FIG. 2A provides a panel of photographic images of ovarian cancer in patient 009-001 showing a complex subcutaneous tumor nest comprising multiple tumors at baseline (top left panel), then at dose C1D15 (top left panel), C2D15 (bottom left panel), and C4D1 (bottom right panel). Primary injected tumor is indicated by a circle and OX40L mRNA injection site is indicated by an arrow.

FIG. 2B provides a panel of transverse abdominal/pelvis CT scan images of the tumor lesions presented by patient 009-001, showing a reduction in tumor size at day 56 post-initial dose (FIG. 2B, right panels) relative to baseline (FIG. 2B, left panels).

FIG. 3A provides a panel of transverse abdominal/pelvis CT scan images of the tumor lesions presented by patient 007-002, showing a reduction in sternal tumor lesion size day ˜56 post-initial dose (FIG. 3A, right panels) relative to baseline (FIG. 3B, left panels).

FIG. 3B provides a photographic image of a sternal ulcer presented by patient 007-002 showing resolution of the underlying injected tumor.

FIG. 4A provides a graph depicting the OX40L expression from pre- and post-treatment tumor biopsies from cancer patients, as indicated, as determined by quantitative immunofluorescent assay (QIF).

FIGS. 4B-4C shows a representative immunofluorescence image showing localized, focal increases in OX40L expression at one day post-C1D1 dose (FIG. 4C) in injected tumor relative to baseline (FIG. 4B) in patient 007-002. (AQ=OX40L AQUA score; DAPI=DNA nuclear stain 4′,6-diamidino-2-phenylindole; CK=cytokeratin).

### DETAILED DESCRIPTION

The present disclosure is directed to methods of treating ovarian cancer, or other cancers such as solid tumors, lymphomas or epithelial origin cancers (e.g., an epithelial cancer of ovary, fallopian tube or peritoneum), in a human patient by administering an effective amount of an mRNA encoding human OX40L polypeptide, alone or in combination with another agent, such as an immune checkpoint inhibitor. In some aspects, the mRNA is encapsulated in a lipid nanoparticle. In some aspects, administering an effective amount of an mRNA encoding human OX40L polypeptide reduces or decreases the size of a tumor (e.g., the tumor which has been injected and/or a proximal, un-injected tumor) in an ovarian, solid tumor, lymphoma or epithelial origin cancer patient. In some aspects, administering an effective amount of an mRNA encoding human OX40L polypeptide induces a specific cell-mediated immune response with systemic anti-tumor effects in an ovarian, solid tumor, lymphoma or epithelial origin cancer patient. In some aspects, the expression of human OX40L in tumor cells and/or in immune cells in the tumor microenvironment is increased after administration of an mRNA encoding human OX40L.

### Methods of Use and Dosing Regimens

In some embodiments, the present disclosure provides methods of administering an mRNA encoding human OX40L polypeptide for treating ovarian cancer, or other cancers such as solid tumors, lymphomas or epithelial origin cancers (e.g., an epithelial cancer of ovary, fallopian tube or peritoneum) in a subject. The most common type of ovarian cancer (about 90% of ovarian cancer) is epithelial in origin. These cancers begin in the thin layer of tissue that covers the outside of the ovaries. Stromal tumors can also occur and result in about 7 percent of ovarian tumors. These tumors begin in the ovarian tissue that contains hormone-producing cells. Germ cells tumors, which begin in egg-producing cells also occur, although rarely.

Clinical diagnosis is based on a biopsy, which is usually performed under computerized tomography scan or ultrasound. The poor outcome of this illness is due in particular to a late diagnosis, due in particular to the absence of early signs and symptoms.

Table 1 summarizes the various stages of ovarian cancer:

TABLE 1
Ovarian Cancer Stages

| AJCC | Stage | FIGO | |
|---|---|---|---|
| Stage | grouping | Stage | Stage description |
| I | T1 | I | The cancer is only in the ovary (or ovaries) or fallopian |
| | N0 | | tube(s) (T1). |
| | M0 | | It has not spread to nearby lymph nodes (N0) or to distant |
| | | | sites (M0). |
| IA | T1a | IA | The cancer is in one ovary, and the tumor is confined to the |
| | N0 | | inside of the ovary; or the cancer is in in one fallopian tube, |
| | M0 | | and is only inside the fallopian tube. There is no cancer on |
| | | | the outer surfaces of the ovary or fallopian tube. No cancer |
| | | | cells are found in the fluid (ascites) or washings from the |
| | | | abdomen and pelvis (T1a). |
| | | | It has not spread to nearby lymph nodes (N0) or to distant |
| | | | sites (M0). |
| IB | T1b | IB | The cancer is in both ovaries or fallopian tubes but not on |
| | N0 | | their outer surfaces. No cancer cells are found in the fluid |
| | M0 | | (ascites) or washings from the abdomen and pelvis (T1b). It |
| | | | has not spread to nearby lymph nodes (N0) or to distant sites |
| | | | (M0). |
| IC | T1c | IC | The cancer is in one or both ovaries or fallopian tubes and |
| | N0 | | any of the following are present: |
| | M0 | | The tissue (capsule) surrounding the tumor broke |
| | | | during surgery, which could allow cancer cells to leak |
| | | | into the abdomen and pelvis (called surgical spill). |
| | | | This is stage IC1. |
| | | | Cancer is on the outer surface of at least one of the |
| | | | ovaries or fallopian tubes or the capsule (tissue |
| | | | surrounding the tumor) has ruptured (burst) before |
| | | | surgery (which could allow cancer cells to spill into |
| | | | the abdomen and pelvis). This is stage IC2. |
| | | | Cancer cells are found in the fluid (ascites) or |
| | | | washings from the abdomen and pelvis. This is |
| | | | stage IC3. |
| | | | It has not spread to nearby lymph nodes (N0) or to distant |
| | | | sites (M0). |
| II | T2 | II | The cancer is in one or both ovaries or fallopian tubes and |
| | N0 | | has spread to other organs (such as the uterus, bladder, the |
| | M0 | | sigmoid colon, or the rectum) within the pelvis or there is |
| | | | primary peritoneal cancer (T2). It has not spread to nearby |
| | | | lymph nodes (N0) or to distant sites (M0). |
| IIA | T2a | IIA | The cancer has spread to or has invaded (grown into) the |
| | N0 | | uterus or the fallopian tubes, or the ovaries. (T2a). It has not |
| | M0 | | spread to nearby lymph nodes (N0) or to distant sites (M0). |
| IIB | T2b | IIB | The cancer is on the outer surface of or has grown into other |
| | N0 | | nearby pelvic organs such as the bladder, the sigmoid colon, |
| | M0 | | or the rectum (T2b). It has not spread to nearby lymph nodes |
| | | | (N0) or to distant sites (M0). |
| IIIA1 | T1 or T2 | IIIA1 | The cancer is in one or both ovaries or fallopian tubes, or |
| | N1 | | there is primary peritoneal cancer (T1) and it may have |
| | M0 | | spread or grown into nearby organs in the pelvis (T2). It has |
| | | | spread to the retroperitoneal (pelvic and/or para-aortic) lymph |
| | | | nodes only. It has not spread to distant sites (M0). |
| IIIA2 | T3a | IIIA2 | The cancer is in one or both ovaries or fallopian tubes, or |
| | N0 or N1 | | there is primary peritoneal cancer and it has spread or grown |
| | M0 | | into organs outside the pelvis. During surgery, no cancer is |
| | | | visible in the abdomen (outside of the pelvis) to the naked |
| | | | eye, but tiny deposits of cancer are found in the lining of the |
| | | | abdomen when it is examined in the lab (T3a). |
| | | | The cancer might or might not have spread to retroperitoneal |
| | | | lymph nodes (N0 or N1), but it has not spread to distant sites |
| | | | (M0). |
| IIIB | T3b | IIIB | There is cancer in one or both ovaries or fallopian tubes, or |
| | N0 or N1 | | there is primary peritoneal cancer and it has spread or grown |
| | M0 | | into organs outside the pelvis. The deposits of cancer are |
| | | | large enough for the surgeon to see, but are no bigger than 2 |
| | | | cm (about ¾ inch) across. (T3b). |
| | | | It may or may not have spread to the retroperitoneal lymph |
| | | | nodes (N0 or N1), but it has not spread to the inside of the |
| | | | liver or spleen or to distant sites (M0). |
| IIIC | T3c | IIIC | The cancer is in one or both ovaries or fallopian tubes, or |
| | N0 or N1 | | there is primary peritoneal cancer and it has spread or grown |
| | M0 | | into organs outside the pelvis. The deposits of cancer are |
| | | | larger than 2 cm (about ¾ inch) across and may be on the |
| | | | outside (the capsule) of the liver or spleen (T3c). |
| | | | It may or may not have spread to the retroperitoneal lymph |
| | | | nodes (N0 or N1), but it has not spread to the inside of the |
| | | | liver or spleen or to distant sites (M0). |
| IVA | Any T | IVA | Cancer cells are found in the fluid around the lungs (called a |
| | Any N | | malignant pleural effusion) with no other areas of cancer |
| | M1a | | spread such as the liver, spleen, intestine, or lymph nodes |
| | | | outside the abdomen (M1a). |
| IVB | Any T | IVB | The cancer has spread to the inside of the spleen or liver, to |
| | Any N | | lymph nodes other than the retroperitoneal lymph nodes, |
| | M1b | | and/or to other organs or tissues outside the peritoneal cavity |
| | | | such as the lungs and bones (M1b). |

Accordingly, ovarian cancers to be treated according to the disclosure include cancers originating in any ovarian tissue, including tumors of ovarian epithelial origin, ovarian stromal origin and ovarian germ cell origin and at any stage, including stage I, IA, IB, IC, II, IIA, IIB, IIIA1, IIIA2, IIIB, IIIC, IVA and IVB ovarian cancers.

In some embodiments, other cancers of epithelial origin, such as epithelial cancers of the ovary or of tissue in the vicinity of the ovary, including the fallopian tube(s) and the peritoneum, are treated according to the disclosure.

Still further solid malignancies and lymphomas are treated according to the disclosure.

In some embodiments, the patient selected for treatment according to the disclosure has undergone one or more cancer treatments prior to treatment with an mRNA of the disclosure or an LNP comprising an mRNA of the disclosure. In some embodiments, the patient to be treated has not responded to at least one prior anti-cancer treatment or at least one prior anti-cancer treatment has become ineffective (e.g., no longer inhibits tumor progression). In one embodiment, the prior anti-cancer treatment is a chemotherapy treatment. In one embodiment, the prior anti-cancer treatment is a radiotherapy treatment. In one embodiment, the prior anti-cancer treatment is an immunotherapy treatment (e.g., treatment with an immune checkpoint inhibitor alone, treatment with an immunostimulatory cytokine, treatment with an anti-cancer vaccine, treatment with CAR-T cell therapy).

In some embodiments, the methods described herein comprise administering to the subject an mRNA encoding human OX40L of the disclosure, or lipid nanoparticles comprising said mRNA, and pharmaceutical compositions comprising said lipid nanoparticle.

Compositions of the disclosure are administered to the subject in an effective amount. In general, an effective amount of the composition will allow for efficient production of the encoded polypeptide in cells of the subject. Metrics for efficiency may include polypeptide translation (indicated by polypeptide expression), level of mRNA degradation, and immune response indicators.

The methods of the disclosure for treating an ovarian cancer, or other cancers such as solid tumors, lymphomas or epithelial origin cancers (e.g., an epithelial cancer of ovary, fallopian tube or peritoneum), are used in a variety of clinical or therapeutic applications. For example, the methods are used to stimulate anti-tumor immune responses in a subject with ovarian cancer, or other cancers such as solid tumors, lymphomas or epithelial origin cancers (e.g., an epithelial cancer of ovary, fallopian tube or peritoneum). The mRNA and compositions of the present disclosure are useful in methods for treating or delaying progression of an ovarian cancer, or other cancers such as solid tumors, lymphomas or epithelial origin cancers (e.g., an epithelial cancer of ovary, fallopian tube or peritoneum), in a subject, e.g., a human patient by intratumoral injection. The injection can be in a single injection at a single site or multiple injections at one or more sites (one or more tumors). The injection can be a bolus injection or a continuous infusion. Preferably, the mRNA is administered in a single injection; however, multiple injections into different sites within the same lesion or split across several lesions are used when no single lesion is available that is large enough to receive the entire dose in the maximum injection volume per lesion size.

A suitable dose of an mRNA is a dose which treats or delays progression of ovarian cancer, or other cancers such as solid tumors, lymphomas or epithelial origin cancers (e.g., an epithelial cancer of ovary, fallopian tube or peritoneum), in a human patient, and may be affected by a variety of factors including, e.g., the age, sex, and weight of a subject to be treated and the particular mRNA to be used. Other factors affecting the dose administered to the subject include, e.g., the type or severity of the ovarian cancer in the patient. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject.

In some embodiments, a subject is administered at least one mRNA composition described herein. In related embodiments, the subject is provided with or administered a nanoparticle (e.g., a lipid nanoparticle) comprising the mRNA(s). In further related embodiments, the subject is provided with or administered a pharmaceutical composition of the disclosure to the subject. In particular embodiments, the pharmaceutical composition comprises an mRNA(s) as described herein, or it comprises a nanoparticle comprising the mRNA(s). In particular embodiments, the mRNA(s) is present in a nanoparticle, e.g., a lipid nanoparticle. In particular embodiments, the mRNA(s) or nanoparticle is present in a pharmaceutical composition, e.g., a composition suitable for intratumoral injection.

In some embodiments, the mRNA(s), nanoparticle, or pharmaceutical composition is administered to the patient parenterally, e.g., intratumorally. In particular embodiments, the subject is a mammal, e.g., a human. In various embodiments, the subject is provided with an effective amount of the mRNA(s).

Suitable doses for human patients can be evaluated in, e.g., a Phase I dose escalation study. Data obtained from the cell culture assays and animal studies can be used in formulating a range of dosage for use in humans. The dosage of such mRNA described herein lies generally within a range of local concentrations of the mRNA that include the ED50 with little or no toxicity. The dosage may vary within this range depending upon the dosage form employed and the route of administration utilized. For the mRNA and compositions described herein, the therapeutically effective dose can be estimated initially from cell culture assays. A dose can be formulated in animal models to achieve a therapeutically effective concentration within the local site that includes the IC50 (i.e., the concentration of the mRNA which achieves a half-maximal inhibition of symptoms) as determined in cell culture. Such information can be used to more accurately determine useful doses in humans.

In some embodiments, the frequency of dosing will take into account the pharmacokinetic parameters of the mRNA in the formulation used. In certain embodiments, a clinician will administer the composition until a dosage is reached that achieves or maintains the desired effect. In some embodiments, the desired effect is tumor size reduction or resolution of the injected or uninjected tumors. In some embodiments, the desired effect is expression of OX40L within the tumor. In some embodiments, achievement of a desired effect occurs immediately after administration of a dose. In some embodiments, achievement occurs at any point in time following administration. In some embodiments, achievement occurs at any point in time during a dosing interval. In some embodiments, achievement of a desired effect is determined by analyzing a biological sample (e.g., tumor biopsy) immediately after administration of a dose, at any point in time following administration of a dose, at any point in time during a doing interval, or combinations thereof.

In some embodiments, maintenance of a desired effect (e.g., OX40L protein expression) is determined by analyzing a biological sample (e.g., tumor biopsy) at least once during a dosing interval. In some embodiments, maintenance of a desired effect (e.g., OX40L protein expression.) is determined by analyzing a biological sample (e.g., tumor biopsy) at regular intervals during a dosing interval. In some embodiments, maintenance of a desired effect (e.g., OX40L protein expression) is determined by analyzing a biological sample (e.g., tumor biopsy) before a subsequent dose is administered.

### Tumor Size Reduction or Growth Inhibition

In some embodiments, the subject having ovarian cancer, or other cancers such as solid tumors, lymphomas or epithelial origin cancers (e.g., an epithelial cancer of ovary, fallopian tube or peritoneum), has a tumor as described supra. In some embodiments, the human OX40L encoding mRNA is administered locally to a tumor (i.e., intratumorally). In some embodiments, administration of the human OX40L encoding mRNA to a tumor reduces the size or volume, or inhibits the growth of the injected tumor. In some embodiments, administration of the human OX40L encoding mRNA to a tumor reduces the size or inhibits the growth of the injected tumor and an uninjected tumor in the subject. In some embodiments, the uninjected tumor is located near or proximal to the injected tumor. In some embodiments, the uninjected tumor is located distal to the injected tumor. In some embodiments, the reduction in size or inhibition of growth in an uninjected tumor is through an abscopal effect.

In some embodiments, reduction in tumor size is by at least 25%. In some embodiments, reduction in tumor size is by at least 50%. In some embodiments, reduction in tumor size is by at least 75%. In some embodiments, the tumor is completely resolved.

In some embodiments, a reduction in tumor size is measured by comparison to the size of patient's tumor at baseline, against an expected tumor size, against an expected tumor size based on a large patient population, or against the tumor size of a control population.

In some embodiments, tumor size is determined by visual methods, such as image scanning. Methods for determining tumor size and tumor volume are known to those of skill in the art.

### OX40L Protein Expression

In some embodiments, human OX40L protein expression is enhanced in a tumor administered an OX40L encoding mRNA. In some embodiments, enhancement of human OX40L protein expression is relative to expression prior to administration of the OX40L encoding mRNA. In some embodiments, a biopsy is obtained from the tumor before and after administration of the OX40L encoding mRNA, and protein expression is assessed.

In some embodiments, human OX40L protein expression is increased by at least 1-fold, 2-fold, 3-fold, 4-fold, 5-fold, 6-fold, 7-fold, 8-fold, 9-fold, or 10-fold, on tumor cells or immune cells within a tumor at any point in time following administration of an OX40L encoding mRNA or composition of the disclosure, as determined by a method described herein. In some embodiments, OX40L protein expression is increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval, as determined by a method described herein. In some embodiments, OX40L protein expression is increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval comprising a duration of 7-35 days, as determined by a method described herein. In some embodiments, OX40L protein expression is increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval comprising a duration of 14-28 days, as determined by a method described herein. In some embodiments, OX40L protein expression is increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval comprising a duration of 21-28 days, as determined by a method described herein. In some embodiments, OX40L protein expression is increased by at least 1-fold, 2-fold, 3-fold, 4-fold, 5-fold, 6-fold, 7-fold, 8-fold, 9-fold, or 10-fold, on tumor cells or immune cells within a tumor on, during or after day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or day 35 of the dosing interval, as determined by a method described herein.

Methods for determining human OX40L protein expression on appropriate cell types, such as immune cells or tumor cells located within a tumor are known to those of skill in the art and described herein. Such methods include, but are not limited to, quantitative immunofluorescence (QIF), flow cytometry, reverse transcription polymerase chain reaction (RT-PCR), competitive RT-PCR, real-time RT-PCR, RNase protection assay (RPA), northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

### Enhancing Anti-Tumor Immune Responses

In some embodiments, the disclosure provides a method for enhancing an immune response in a subject with ovarian cancer, or other cancers such as solid tumors, lymphomas or epithelial origin cancers (e.g., an epithelial cancer of ovary, fallopian tube or peritoneum). In some embodiments, the disclosure provides a method for enhancing an immune response to an ovarian tumor, or other cancers such as solid tumors, lymphomas or epithelial origin cancers (e.g., an epithelial cancer of ovary, fallopian tube or peritoneum). In some embodiments, enhancing an immune response comprises stimulating cytokine production. In another embodiment, enhancing an immune response comprises enhancing cellular immunity (T cell responses), such as activating T cells. In some embodiments, enhancing an immune response comprises activating NK cells. Enhancement of an immune response in a subject can be evaluated by a variety of methods established in the art for assessing immune response, including but not limited to determining the level of T cell activation and NK cell activation by intracellular staining of activation markers in the area of the tumor.

In some embodiments, local administration of an mRNA encoding a human OX40L polypeptide to a tumor induces T cell activation within the tumor. In some embodiments, the activation of T cells results in an anti-tumor immune response in the subject. In some embodiments, the activated T cells in the subject reduce or decrease the size of a tumor or inhibit the growth of a tumor in the subject. Activation of T cells can be measured using applications in the art such as measuring T cell proliferation; measuring cytokine production with enzyme-linked immunosorbant assays (ELISA) or enzyme-linked immunospot assays (ELISPOT); or detection of cell-surface markers associated with T cell activation (e.g., CD69, CD40L, CD137, CD25, CD71, CD26, CD27, CD28, CD30, CD154, and CD134) with techniques such as flow cytometry.

In some embodiments, the activated T cells are CD4⁺ cells, CD8⁺ cells, CD62⁺ (L-selectin⁺) cells, CD69⁺ cells, CD40L⁺ cells, CD137⁺ cells, CD25⁺ cells, CD71⁺ cells, CD26⁺ cells, CD27⁺ cells, CD28⁺ cells, CD30⁺ cells, CD45⁺ cells, CD45RA⁺ cells, CD45RO⁺ cells, CD11b⁺ cells, CD154⁺ cells, CD134⁺ cells, CXCR3⁺ cells, CCR4⁺ cells, CCR6⁺ cells, CCR7⁺ cells, CXCR5⁺ cells, Crth2⁺ cells, gamma delta T cells, or any combination thereof. In some embodiments, the activated T cells are Th₁ cells. In other embodiments, the T cells are Th₂ cells. In other embodiments, the activated T cells activated are cytotoxic T cells.

In some embodiments, local administration of an mRNA encoding a human OX40L polypeptide to a tumor induces T cell proliferation within the tumor. In some embodiments, T cell proliferation results in an anti-tumor immune response in the subject. In some embodiments, T cell proliferation in the subject reduce or decrease the size of a tumor or inhibit the growth of a tumor in the subject. T cell proliferation can be measured using applications in the art such as cell counting, viability staining, optical density assays, or detection of cell-surface markers associated with T cell activation (e.g., CD69, CD40L, CD137, CD25, CD71, CD26, CD27, CD28, CD30, CD154, and CD134) with techniques such as flow cytometry.

In some embodiments, local administration of an mRNA encoding a human OX40L polypeptide to a tumor induces infiltration of T cells to the tumor. In some embodiments, T cell infiltration results in an anti-tumor immune response in the subject. In some embodiments, T cell infiltration in the subject reduce or decrease the size of a tumor or inhibit the growth of a tumor in the subject. T cell infiltration in a tumor can be measured using applications in the art such as tissue sectioning and staining for cell markers, measuring local cytokine production at the tumor site, or detection of T cell-surface markers with techniques such as flow cytometry.

In some embodiments, the infiltrating T cells are CD4⁺ cells, CD8⁺ cells, CD62⁺ (L-selectin⁺) cells, CD69⁺ cells, CD40L⁺ cells, CD137⁺ cells, CD25⁺ cells, CD71⁺ cells, CD26⁺ cells, CD27⁺ cells, CD28⁺ cells, CD30⁺ cells, CD45⁺ cells, CD45RA⁺ cells, CD45R0⁺ cells, CD11b⁺ cells, CD154⁺ cells, CD134⁺ cells, CXCR3⁺ cells, CCR4⁺ cells, CCR6⁺ cells, CCR7⁺ cells, CXCR5⁺ cells, Crth2⁺ cells, gamma delta T cells, or any combination thereof. In some embodiments, the infiltrating T cells are Th₁ cells. In other embodiments, the infiltrating T cells are Th₂ cells. In other embodiments, the infiltrating T cells are cytotoxic T cells.

In some embodiments, local administration of an mRNA encoding a human OX40L polypeptide to a tumor increases the number of Natural Killer (NK) cells within the tumor. In some embodiments, the increase in the number of NK cells results in an anti-tumor immune response in the subject. In some embodiments, the increase in the number of NK cells reduces or decreases the size of a tumor or inhibits the growth of a tumor in the subject. Increases in the number of NK cells in a subject can be measured using applications in the art such as detection of NK cell-surface markers (e.g., CD335/NKp46; CD336/NKp44; CD337/NPp30) or intracellular NK cell markers (e.g., perforin; granzymes; granulysin).

In some embodiments, administration of the mRNA encoding an OX40L polypeptide increases the total number of NK cells in the tumor compared to the number of NK cells in a tumor that is not administered with the mRNA encoding an OX40L polypeptide.

In certain embodiments, the number of NK cells is increased at least about two-fold, at least about three-fold, at least about four-fold, at least about five-fold, at least about six-fold, at least about seven-fold, at least about eight-fold, at least about nine-fold, or at least about ten-fold compared to a control (e.g., saline or an mRNA without OX40L expression).

Dosing

In some embodiments, the mRNA or composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In some embodiments, the dosing regimen is determined by the pharmacodynamics effects of the human OX40L polypeptide. In some embodiments, the pharmacodynamics effects include an increase in T cells within tumors after administration. In some embodiments, the increase in T cells is maintained over a specified period of time (e.g., 14 days).

In some embodiments, the composition comprising an mRNA encoding human OX40L is administered at a dosing interval comprising a duration of about 14-28 days or about 21-28 days. In some embodiments, the composition comprising an mRNA encoding human OX40L is administered at a dosing interval comprising a duration of about 7-42 days, about 7-21 days, about 14-28 days, about 21-28 days, about 21-35 days, about 28-35 days, about 21-42 days, or about 28-42 days. In some embodiments, the dosing interval is about 7 days. In some embodiments, the dosing interval is about 14 days. In some embodiments, the dosing interval is about 21 days. In some embodiments, the dosing interval is about 28 days. In some embodiments, the dosing interval is about 35 days. In some embodiments, the dosing interval is about 42 days. In some embodiments, the dosing interval is at least about 7 days. In some embodiments, the dosing interval is at least about 14 days. In some embodiments, the dosing interval is at least about 21 days. In some embodiments, the dosing interval is at least about 28 days. In some embodiments, the dosing interval is at least about 35 days. In some embodiments, the dosing interval is at least about 42 days.

In some embodiments, the composition comprising an mRNA encoding human OX40L is administered to a subject about every 7-42 days for a specified time period. In some embodiments, the composition comprising an mRNA encoding human OX40L is administered to a subject about every 7-21 days for a specified time period. In some embodiments, the composition comprising an mRNA encoding human OX40L is administered to a subject about every 14-21 days for a specified time period. In some embodiments, the composition comprising an mRNA encoding human OX40L is administered to a subject about every 14-28 days for a specified time period. In some embodiments, the composition comprising an mRNA encoding human OX40L is administered to a subject about every 21-28 days for a specified time period. In some embodiments, the composition comprising an mRNA encoding human OX40L is administered to a subject about every 21-35 days for a specified time period. In some embodiments, the composition comprising an mRNA encoding human OX40L is administered to a subject about every 28-42 days for a specified time period.

In some embodiments, the composition is administered to a subject about every 7 days for a specified time period. In some embodiments, the composition is administered to a subject about every 14 days for a specified time period. In some embodiments, the composition is administered to a subject about every 21 days for a specified time period. In some embodiments, the composition is administered to a subject about every 28 days for a specified time period. In some embodiments, the composition is administered to a subject about every 35 days for a specified time period. In some embodiments, the composition is administered to a subject about every 42 days for a specified time period.

In some embodiments, the specified time period is determined by a clinician. In some embodiments, dosing occurs until a positive therapeutic outcome is achieved. For example, in some embodiments, dosing occurs until growth of cancer cells, tumor cells or tumors is inhibited. In some embodiments, dosing occurs until growth of cancer cells, tumor cells or tumors is reduced. In some embodiments, dosing occurs until there is no detection of cancer cells, tumor cells or tumors in a biological sample. In some embodiments, dosing occurs until progression of a cancer is delayed. In some embodiments, dosing occurs until progression of a cancer is inhibited. In some embodiments, the specified time period is determined once a positive therapeutic outcome is achieved.

In some embodiments, dosing of a composition comprising an mRNA encoding human OX40L will occur indefinitely, or until a positive therapeutic outcome is achieved. In some embodiments, the dosing interval remains consistent. In some embodiments, the dosing interval changes as needed based on a clinician's assessment. In some embodiments, dosing occurs indefinitely to maintain remission of a cancer.

In some embodiments, the composition comprising an mRNA encoding human OX40L is administered to a subject about every 7-42 days indefinitely, or until a positive therapeutic outcome is achieved. In some embodiments, the composition comprising an mRNA encoding human OX40L is administered to a subject about every 7-21 days indefinitely, or until a positive therapeutic outcome is achieved. In some embodiments, the composition comprising an mRNA encoding human OX40L is administered to a subject about every 14-21 days indefinitely, or until a positive therapeutic outcome is achieved. In some embodiments, the composition comprising an mRNA encoding human OX40L is administered to a subject about every 14-28 days indefinitely, or until a positive therapeutic outcome is achieved. In some embodiments, the composition comprising an mRNA encoding human OX40L is administered to a subject about every 21-28 days indefinitely, or until a positive therapeutic outcome is achieved. In some embodiments, the composition comprising an mRNA encoding human OX40L is administered to a subject about every 21-35 days indefinitely, or until a positive therapeutic outcome is achieved. In some embodiments, the composition comprising an mRNA encoding human OX40L is administered to a subject about every 28-42 days indefinitely, or until a positive therapeutic outcome is achieved.

In some embodiments, the composition is administered to a subject about every 7 days indefinitely, or until a positive therapeutic outcome is achieved. In some embodiments, the composition is administered to a subject about every 14 days indefinitely, or until a positive therapeutic outcome is achieved. In some embodiments, the composition is administered to a subject about every 21 days indefinitely, or until a positive therapeutic outcome is achieved. In some embodiments, the composition is administered to a subject about every 28 days indefinitely, or until a positive therapeutic outcome is achieved. In some embodiments, the composition is administered to a subject about every 35 days indefinitely, or until a positive therapeutic outcome is achieved. In some embodiments, the composition is administered to a subject about every 42 days indefinitely, or until a positive therapeutic outcome is achieved.

In some embodiments, the composition is administered on days 1 and 15 (±2 days) of a 28 day cycle for multiple cycles (e.g., 6 cycles total) or until a positive therapeutic outcome is achieved. In some embodiments, the composition is administered on days 1 and 15 (±2 days) of cycle 1 of a 28 day cycle and then on day 1 for multiple subsequent 28 day cycles (e.g., 5 more cycles for a total of 6 cycles) or until a positive therapeutic outcome is achieved.

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the disclosure may be administrated.

In some embodiments, an mRNA composition is administered at a dose between about 0.010 mg/kg to about 0.5 mg/kg for a human patient. In some embodiments, the mRNA composition is administered at a dose between about 0.015 mg/kg to about 0.4 mg/kg. In some embodiments, the mRNA composition is administered at a dose between about 0.020 mg/kg to about 0.3 mg/kg. In some embodiments, the mRNA composition is administered at a dose between about 0.025 mg/kg to about 0.2 mg/kg. In some embodiments, the mRNA composition is administered at a dose between about 0.030 mg/kg to about 0.1 mg/kg.

In some embodiments, an mRNA composition is administered at a dose between about 0.5 mg to about 10.0 mg of mRNA for a human patient. In some embodiments, a composition is administered at a dose of 1.0 mg. In some embodiments, a composition is administered at a dose of 2.0 mg. In some embodiments, a composition is administered at a dose of 4.0 mg. In some embodiments, a composition is administered at a dose of 8.0 mg.

In some embodiments, the composition is administered at a dose of 8.0 mg mRNA on days 1 and 15 (±2 days) of a 28 day cycle for multiple cycles (e.g., 6 cycles total) or until a positive therapeutic outcome is achieved. In some embodiments, the composition is administered at a dose of 8.0 mg RNA on days 1 and 15 (±2 days) of cycle 1 of a 28 day cycle and then on day 1 for multiple subsequent 28 day cycles (e.g., 5 more cycles for a total of 6 cycles) or until a positive therapeutic outcome is achieved.

In some embodiments, a single dose may be administered, for example, prior to or after, or in lieu of a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

### Combination Therapy

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

Exemplary therapeutic agents that may be administered in combination with the compositions of the disclosure include, but are not limited to, cytotoxic, chemotherapeutic, hypomethylating agents, pro-apoptotic agents, small molecules/kinase inhibitors, immunostimulatory agents and other therapeutic agents including therapeutics approved for ovarian cancer, now or at a later date. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

In some embodiments, the human OX40L encoding mRNA is administered to a subject having ovarian cancer, or other cancers such as solid tumors, lymphomas or epithelial origin cancers (e.g., an epithelial cancer of ovary, fallopian tube or peritoneum), wherein the subject has received or is receiving treatment with one or more anti-cancer agents. In some embodiments, the human OX40L encoding mRNA is administered in combination with one or more anti-cancer agents to a subject having ovarian cancer, or other cancers such as solid tumors, lymphomas or epithelial origin cancers (e.g., an epithelial cancer of ovary, fallopian tube or peritoneum). In some embodiments, the OX40L encoding mRNA and one or more anti-cancer agents are administered simultaneously or sequentially. In some embodiments, the OX40L encoding mRNA is administered after administration of one or more anti-cancer agents. In some embodiments, the OX40L encoding mRNA is administered before administration of one or more anti-cancer agents.

In some embodiments, the one or more anti-cancer agents are approved by the United States Food and Drug Administration. In other embodiments, the one or more anti-cancer agents are pre-approved by the United States Food and Drug Administration.

In some embodiments, the subject for the present methods has been treated with one or more standard of care therapies. In other embodiments, the subject for the present methods has not been responsive to one or more standard of care therapies or anti-cancer therapies.

In some embodiments, the subject has been previously treated with a PD-1 antagonist prior to an OX40L encoding mRNA. In some embodiments, the subject is treated with a monoclonal antibody that binds to PD-1 simultaneously with or subsequent to an OX40L encoding mRNA. In some embodiments, the subject has been treated with an anti-PD-1 monoclonal antibody therapy prior to an OX40L encoding mRNA. In some embodiments, the anti-PD-1 monoclonal antibody therapy comprises Nivolumab, Pembrolizumab, Pidilizumab, or any combination thereof.

In some embodiments, the anti-PD-1 antibody (or an antigen-binding portion thereof) useful for the disclosure is pembrolizumab. Pembrolizumab (also known as KEYTRUDA®, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. No. 8,900,587. Pembrolizumab has been approved by the FDA for the treatment of relapsed or refractory melanoma and advanced NSCLC.

In some embodiments, the anti-PD-1 antibody useful for the disclosure is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). Nivolumab has shown activity in a variety of advanced solid tumors including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223.

In other embodiments, the anti-PD-1 antibody is MEDI0680 (formerly AMP-514), which is a monoclonal antibody against the PD-1 receptor. MEDI0680 is described, for example, in U.S. Pat. No. 8,609,089B2.

In some embodiments, the anti-PD-1 antibody is BGB-A317, which is a humanized monoclonal antibody. BGB-A317 is described in U.S. Publ. No. 2015/0079109.

In some embodiments, a PD-1 antagonist is AMP-224, which is a B7-DC Fc fusion protein. AMP-224 is discussed in U.S. Publ. No. 2013/0017199.

In some embodiments, the subject has been treated with a monoclonal antibody that binds to PD-L1 prior to an OX40L encoding mRNA. In some embodiments, the subject has been treated with an anti-PD-L1 monoclonal antibody therapy simultaneously with or subsequent to an OX40L encoding mRNA. In some embodiments, the anti-PD-L1 monoclonal antibody therapy comprises Durvalumab, Avelumab, MEDI473, BMS-936559, Atezolizumab, or any combination thereof. In some embodiments, the anti-PD-L1 monoclonal antibody therapy comprises Durvalumab.

In some embodiments, the anti-PD-L1 antibody useful for the disclosure is MSB0010718C (also called Avelumab; See US 2014/0341917) or BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446) (see, e.g., Herbst et al. (2013) J Clin Oncol 31(suppl):3000. Abstract; U.S. Pat. No. 8,217,149), MEDI4736 (also called Durvalumab; Khleif (2013) In: Proceedings from the European Cancer Congress 2013; Sep. 27-Oct. 1, 2013; Amsterdam, The Netherlands.

In some embodiments, the subject has been treated with a CTLA-4 antagonist prior to an OX40L encoding mRNA. In some embodiments, the subject has been previously treated with a monoclonal antibody that binds to CTLA-4 prior to an OX40L encoding mRNA. In some embodiments, the subject has been treated with an anti-CTLA-4 monoclonal antibody simultaneously with or subsequent to an OX40L encoding mRNA. In other aspects, the anti-CTLA-4 antibody therapy comprises Ipilimumab or Tremelimumab.

An exemplary clinical anti-CTLA-4 antibody is the human mAb 10D1 (now known as ipilimumab and marketed as YERVOY®) as disclosed in U.S. Pat. No. 6,984,720. Another anti-CTLA-4 antibody useful for the present methods is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

In some embodiments, an mRNA therapeutic of the invention is administered to a subject having ovarian cancer, or other cancers such as solid tumors, lymphomas or epithelial origin cancers (e.g., an epithelial cancer of ovary, fallopian tube or peritoneum), in combination with a checkpoint inhibitor.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### Messenger RNA Encoding OX40L

The mRNAs of the present application encode an OX40L polypeptide. OX40L is the ligand for OX40 (CD134). OX40L has also been designated CD252 (cluster of differentiation 252), tumor necrosis factor (ligand) superfamily, member 4, tax-transcriptionally activated glycoprotein 1, TXGP1, or gp34. Human OX40L is 183 amino acids in length and contains three domains: a cytoplasmic domain of amino acids 1-23; a transmembrane domain of amino acids 24-50, and an extracellular domain of amino acids 51-183.

Human OX40L was first identified on the surface of human lymphocytes infected with human T-cell leukemia virus type-I (HTLV-I) by Tanaka et al. (Tanaka et al., International Journal of Cancer (1985), 36(5):549-55). Human OX40L is a 34 kDa glycosylated type II transmembrane protein that exists on the surface of cells as a trimer. OX40L comprises a cytoplasmic domain (amino acids 1-23), a transmembrane domain (amino acids 24-50) and an extracellular domain (amino acids 51-183). OX40L is also referred to as Tumor Necrosis Factor Superfamily (ligand) Member 4 (TNFSF4), CD252, CD134L, Tax-Transcriptionally Activated Glycoprotein 1 (TXGP1), Glycoprotein 34 (GP34), and ACT-4-L.

In some embodiments, a composition suitable for use in the methods of the disclosure comprises an mRNA encoding a mammalian OX40L polypeptide. In some embodiments, the mammalian OX40L polypeptide is a murine OX40L polypeptide. In some embodiments, the mammalian OX40L polypeptide is a human OX40L polypeptide. In some embodiments, the OX40L polypeptide comprises an amino acid sequence set forth in SEQ ID NOs: 1-3.

In some embodiments, the mRNA encoding a human OX40L polypeptide encodes a human OX40L polypeptide comprising an amino acid sequence at least 50%, at least 60%, at least 70%, at least 80%, at least 85%, at least 90%, at least 95%, at least 98%, at least 99%, or 100% identical to an amino acid sequence set forth in SEQ ID NOs: 1-3 or an amino acid sequence encoded by a nucleotide sequence set forth in SEQ ID NOs: 4-10, wherein the human OX40L polypeptide is capable of binding to an OX40 receptor. In some embodiments, the mRNA encoding a human OX40L polypeptide encodes a human OX40L polypeptide comprising an amino acid sequence at least 50%, at least 60%, at least 70%, at least 80%, at least 85%, at least 90%, at least 95%, at least 98%, at least 99%, or 100% identical to SEQ ID NO: 1 and is capable of binding to an OX40 receptor. In some embodiments, the mRNA encoding a human OX40L polypeptide encodes a human OX40L polypeptide that consists essentially of SEQ ID NO: 1 and is capable of binding to an OX40 receptor.

In certain embodiments, the mRNA encoding a human OX40L polypeptide encodes a human OX40L polypeptide comprising an amino acid sequence set forth in SEQ ID NOs: 1-3, optionally with one or more conservative substitutions, wherein the conservative substitutions do not significantly affect the binding activity of the OX40L polypeptide to its receptor, i.e., the OX40L polypeptide binds to the OX40 receptor after the substitutions. In some embodiments, the mRNA encoding a human OX40L polypeptide encodes a human OX40L polypeptide comprising an amino acid sequence having at least 80%, at least 85%, at least 90%, at least 95%, at least 99% or 100% identity to any one of the amino acid sequences set forth in SEQ ID NOs: 1-3.

In other embodiments, an mRNA encoding a human OX40L polypeptide comprises a nucleotide sequence at least 50%, at least 60%, at least 70%, at least 80%, at least 85%, at least 90%, at least 95%, at least 98%, at least 99%, or 100% identical to any one of the nucleic acid sequences set forth in SEQ ID NOs: 4-10. In some embodiments, the mRNA encoding a human OX40L polypeptide comprises an open reading frame comprising a nucleotide sequence selected from any one of SEQ ID NOs: 4 and 8-10. In some embodiments, the mRNA encoding a human OX40L polypeptide comprises an open reading frame comprising a nucleotide sequence having at least 80%, at least 85%, at least 90%, at least 95%, at least 99%, or 100% identity to a nucleotide sequence selected from any one of SEQ ID NOs: 4 and 8-10. In some embodiments, the mRNA encoding a human OX40L polypeptide comprises an open reading frame comprising the nucleotide sequence set forth in SEQ ID NO: 4. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 4. In some embodiments, the mRNA encoding a human OX40L polypeptide comprises an open reading frame comprising the nucleotide sequence set forth in SEQ ID NO: 8. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 8. In some embodiments, the mRNA encoding a human OX40L polypeptide comprises an open reading frame comprising the nucleotide sequence set forth in SEQ ID NO: 9. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 9. In some embodiments, the mRNA encoding a human OX40L polypeptide comprises an open reading frame comprising the nucleotide sequence set forth in SEQ ID NO: 10. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 10.

In some embodiments, the mRNA useful for the methods and compositions described herein comprises an open reading frame encoding an extracellular domain of OX40L. In other embodiments, the mRNA comprises an open reading frame encoding a cytoplasmic domain of OX40L. In some embodiments, the mRNA comprises an open reading frame encoding a transmembrane domain of OX40L. In certain embodiments, the mRNA comprises an open reading frame encoding an extracellular domain of OX40L and a transmembrane domain of OX40L. In other embodiments, the mRNA comprises an open reading frame encoding an extracellular domain of OX40L and a cytoplasmic domain of OX40L. In yet other embodiments, the mRNA comprises an open reading frame encoding an extracellular domain of OX40L, a transmembrane of OX40L, and a cytoplasmic domain of OX40L.

A person of skill in the art would understand that in addition to the native signal sequences and propeptide sequences implicitly disclosed in SEQ ID NOs: 1-10 (sequences present in the precursor form and absent in the mature corresponding form) and non-native signal peptides, other signal sequences can be used. Accordingly, references to OX40L polypeptide or mRNA according to SEQ ID NOs: 1-10 encompass variants in which an alternative signal peptide (or encoding sequence) known in the art has been attached to said OX40L polypeptide (or mRNA). It is also understood that references to the sequences disclosed in SEQ ID NOs: 1-10 through the application are equally applicable and encompass orthologs and functional variants (for example polymorphic variants) and isoforms of those sequences known in the art at the time the application was filed.

In some embodiments, the OX40L encoding mRNA comprises an open reading frame encoding OX40L, a 3′UTR, and a 5′UTR. In some embodiments, the OX40L encoding mRNA comprises an open reading frame encoding OX40L, a 3′UTR, a 5′UTR, and a poly-A tail. In some embodiments, the OX40L encoding mRNA comprises an open reading frame encoding OX40L, a 3′UTR, a 5′UTR, a poly-A tail and a 5′cap.

In some embodiments, the OX40L encoding mRNA comprises (i) a 5′UTR comprising the nucleotide sequence set forth in SEQ ID NO: 15; (ii) an open reading frame encoding OX40L comprising the nucleotide sequence set forth in SEQ ID NO: 4; and (iii) a 3′UTR comprising the nucleotide sequence set forth in SEQ ID NO: 17.

In some embodiments, the OX40L encoding mRNA comprises the nucleotide sequence set forth in SEQ ID NO: 5. In some embodiments, the OX40L encoding mRNA comprises a nucleotide sequence having at least 80%, 85%, 90%, 95%, 96%, 97%, 98% or 99% identity to the nucleotide sequence set forth in SEQ ID NO: 5.

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 15. Another exemplary 5′ UTR for use in the constructs is shown in SEQ ID NO: 16. Another exemplary 5′ UTR for use in the constructs is shown in SEQ ID NO: 12. An exemplary 3′ UTR for use in the constructs is shown in SEQ ID NO: 17. An exemplary 3′ UTR comprising miR-122 and miR-142.3p binding sites for use in the constructs is shown in SEQ ID NO: 18. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3′}GpppG, m₂^{7,O3′}GppppG, m₂^{7,O2′}GppppG, m⁷Gpppm⁷G, m⁷3′dGpppG, m₂^{7,O3′}GpppG, m₂^{7,O3′}GppppG, and m₂^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

### MicroRNA Binding Sites

In some embodiments, the OX40L encoding mRNA comprises one or more microRNA binding sites. microRNAs (or miRNA) are 19-25 nucleotides long noncoding RNAs that bind to the 3′UTR of nucleic acid molecules and down-regulate gene expression either by reducing nucleic acid molecule stability or by inhibiting translation.

By engineering microRNA target sequences into an mRNA, one can target the molecule for degradation or reduced translation, provided the microRNA in question is available. In some embodiments, the miRNA binding site (e.g., miR-122 binding site) binds to the corresponding mature miRNA that is part of an active RNA-induced silencing complex (RISC) containing Dicer. In some embodiments, binding of the miRNA binding site to the corresponding miRNA in RISC degrades the mRNA containing the miRNA binding site or prevents the mRNA from being translated.

Some microRNAs, e.g., miR-122, are abundant in normal tissue but are present in much lower levels in cancer or tumor tissue. Thus, engineering microRNA target sequences (i.e., microRNA binding site) into the OX40L encoding mRNA (e.g., in a 3′UTR like region or other region) can effectively target the molecule for degradation or reduced translation in normal tissue (where the microRNA is abundant) while providing high levels of translation in the cancer or tumor tissue (where the microRNA is present in much lower levels). This provides a tumor-targeting approach for the methods and compositions of the disclosure.

In some embodiments, the microRNA binding site (e.g., miR-122 binding site) is fully complementary to miRNA (e.g., miR-122), thereby degrading the mRNA fused to the miRNA binding site. In other embodiments, the miRNA binding site is not fully complementary to the corresponding miRNA. In certain embodiments, the miRNA binding site (e.g., miR-122 binding site) is the same length as the corresponding miRNA (e.g., miR-122). In other embodiments, the microRNA binding site (e.g., miR-122 binding site) is one nucleotide shorter than the corresponding microRNA (e.g., miR-122, which has 22 nts) at the 5′ terminus, the 3′ terminus, or both. In still other embodiments, the microRNA binding site (e.g., miR-122 binding site) is two nucleotides shorter than the corresponding microRNA (e.g., miR-122) at the 5′ terminus, the 3′ terminus, or both. In yet other embodiments, the microRNA binding site (e.g., miR-122 binding site) is three nucleotides shorter than the corresponding microRNA (e.g., miR-122) at the 5′ terminus, the 3′ terminus, or both. In some embodiments, the microRNA binding site (e.g., miR-122 binding site) is four nucleotides shorter than the corresponding microRNA (e.g., miR-122) at the 5′ terminus, the 3′ terminus, or both. In other embodiments, the microRNA binding site (e.g., miR-122 binding site) is five nucleotides shorter than the corresponding microRNA (e.g., miR-122) at the 5′ terminus, the 3′ terminus, or both. In some embodiments, the microRNA binding site (e.g., miR-122 binding site) is six nucleotides shorter than the corresponding microRNA (e.g., miR-122) at the 5′ terminus, the 3′ terminus, or both. In other embodiments, the microRNA binding site (e.g., miR-122 binding site) is seven nucleotides shorter than the corresponding microRNA (e.g., miR-122) at the 5′ terminus or the 3′ terminus. In other embodiments, the microRNA binding site (e.g., miR-122 binding site) is eight nucleotides shorter than the corresponding microRNA (e.g., miR-122) at the 5′ terminus or the 3′ terminus. In other embodiments, the microRNA binding site (e.g., miR-122 binding site) is nine nucleotides shorter than the corresponding microRNA (e.g., miR-122) at the 5′ terminus or the 3′ terminus. In other embodiments, the microRNA binding site (e.g., miR-122 binding site) is ten nucleotides shorter than the corresponding microRNA (e.g., miR-122) at the 5′ terminus or the 3′ terminus. In other embodiments, the microRNA binding site (e.g., miR-122 binding site) is eleven nucleotides shorter than the corresponding microRNA (e.g., miR-122) at the 5′ terminus or the 3′ terminus. In other embodiments, the microRNA binding site (e.g., miR-122 binding site) is twelve nucleotides shorter than the corresponding microRNA (e.g., miR-122) at the 5′ terminus or the 3′ terminus. The miRNA binding sites that are shorter than the corresponding miRNAs are still capable of degrading the mRNA incorporating one or more of the miRNA binding sites or preventing the mRNA from translation.

In some embodiments, the microRNA binding site (e.g., miR-122 binding site) has sufficient complementarity to miRNA (e.g., miR-122) so that a RISC complex comprising the miRNA (e.g., miR-122) cleaves the polynucleotide comprising the microRNA binding site. In other embodiments, the microRNA binding site (e.g., miR-122 binding site) has imperfect complementarity so that a RISC complex comprising the miRNA (e.g., miR-122) induces instability in the polynucleotide comprising the microRNA binding site. In another embodiment, the microRNA binding site (e.g., miR-122 binding site) has imperfect complementarity so that a RISC complex comprising the miRNA (e.g., miR-122) represses transcription of the polynucleotide comprising the microRNA binding site. In one embodiment, the miRNA binding site (e.g., miR-122 binding site) has one mismatch from the corresponding miRNA (e.g., miR-122). In another embodiment, the miRNA binding site has two mismatches from the corresponding miRNA. In other embodiments, the miRNA binding site has three mismatches from the corresponding miRNA. In other embodiments, the miRNA binding site has four mismatches from the corresponding miRNA. In some embodiments, the miRNA binding site has five mismatches from the corresponding miRNA. In other embodiments, the miRNA binding site has six mismatches from the corresponding miRNA. In certain embodiments, the miRNA binding site has seven mismatches from the corresponding miRNA. In other embodiments, the miRNA binding site has eight mismatches from the corresponding miRNA. In other embodiments, the miRNA binding site has nine mismatches from the corresponding miRNA. In other embodiments, the miRNA binding site has ten mismatches from the corresponding miRNA. In other embodiments, the miRNA binding site has eleven mismatches from the corresponding miRNA. In other embodiments, the miRNA binding site has twelve mismatches from the corresponding miRNA.

In certain embodiments, the miRNA binding site (e.g., miR-122 binding site) has at least about ten contiguous nucleotides complementary to at least about ten contiguous nucleotides of the corresponding miRNA (e.g., miR-122), at least about eleven contiguous nucleotides complementary to at least about eleven contiguous nucleotides of the corresponding miRNA, at least about twelve contiguous nucleotides complementary to at least about twelve contiguous nucleotides of the corresponding miRNA, at least about thirteen contiguous nucleotides complementary to at least about thirteen contiguous nucleotides of the corresponding miRNA, or at least about fourteen contiguous nucleotides complementary to at least about fourteen contiguous nucleotides of the corresponding miRNA. In some embodiments, the miRNA binding sites have at least about fifteen contiguous nucleotides complementary to at least about fifteen contiguous nucleotides of the corresponding miRNA, at least about sixteen contiguous nucleotides complementary to at least about sixteen contiguous nucleotides of the corresponding miRNA, at least about seventeen contiguous nucleotides complementary to at least about seventeen contiguous nucleotides of the corresponding miRNA, at least about eighteen contiguous nucleotides complementary to at least about eighteen contiguous nucleotides of the corresponding miRNA, at least about nineteen contiguous nucleotides complementary to at least about nineteen contiguous nucleotides of the corresponding miRNA, at least about twenty contiguous nucleotides complementary to at least about twenty contiguous nucleotides of the corresponding miRNA, or at least about twenty one contiguous nucleotides complementary to at least about twenty one contiguous nucleotides of the corresponding miRNA.

In some embodiments, an OX40L encoding mRNA comprises at least one miR-122 binding site, at least two miR-122 binding sites, at least three miR-122 binding sites, at least four miR-122 binding sites, or at least five miR-122 binding sites. In some embodiments, the miRNA binding site binds miR-122 or is complementary to miR-122. In some embodiments, the miRNA binding site binds to miR-122-3p or miR-122-5p. In some embodiments, the miRNA binding site comprises a nucleotide sequence at least 80%, at least 85%, at least 90%, at least 95%, or 100% identical to SEQ ID NO: 14, wherein the miRNA binding site binds to miR-122. In another particular aspect, the miRNA binding site comprises a nucleotide sequence at least 80%, at least 85%, at least 90%, at least 95%, or 100% identical to SEQ ID NO: 20, wherein the miRNA binding site binds to miR-122. These sequences are shown below in Table 2.

TABLE 2
miR-122 and miR-122 binding sites

| SEQ ID NO. | Description | Sequence |
|---|---|---|
| SEQ ID NO: 12 | miR-122 | CCUUAGCAGAGCUGUGGAGUGU |
| | | GACAAUGGUGUUUGUGUCUAAA |
| | | CUAUCAAACGCCAUUAUCACAC |
| | | UAAAUAGCUACUGCUAGGC |
| SEQ ID NO: 13 | miR-122-3p | AACGCCAUUAUCACACUAAAUA |
| SEQ ID NO: 14 | miR-122-3p | UAUUUAGUGUGAUAAUGGCGUU |
| | binding site | |
| SEQ ID NO: 19 | miR-122-5p | UGGAGUGUGACAAUGGUGUUUG |
| SEQ ID NO: 20 | miR-122-5p | CAAACACCAUUGUCACACUCCA |
| | binding site | |

In some embodiments, a miRNA binding site (e.g., miR-122 binding site) is inserted in the mRNA in any position (e.g., 3′ UTR); the insertion site in the mRNA can be anywhere in the mRNA as long as the insertion of the miRNA binding site in the mRNA does not interfere with the translation of the functional OX40L polypeptide in the absence of the corresponding miRNA (e.g., miR122); and in the presence of the miRNA (e.g., miR122), the insertion of the miRNA binding site in the mRNA and the binding of the miRNA binding site to the corresponding miRNA are capable of degrading the mRNA or preventing the translation of the mRNA. In one embodiment, a miRNA binding site is inserted in a 3′UTR of the mRNA.

In certain embodiments, a miRNA binding site is inserted in at least about 30 nucleotides downstream from the stop codon of the OX40L encoding mRNA. In other embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of the OX40L encoding mRNA. In other embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of the OX40L encoding mRNA.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (w), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-0H-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-0-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-0H-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂ G), N2,7-dimethyl-guanosine (m^{2,7}G), N2,N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²2 Gm), 1-methyl-2′-O-methyl-guanosine (m¹Gm), N2,7-dimethyl-2′-O-methyl-guanosine (m²′⁷Gm), 2′-O-methyl-inosine (Im), 1,2′-O-dimethyl-inosine (m¹Im), 2′-O-ribosylguanosine (phosphate) (Gr(p)), 1-thio-guanosine, O6-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (w), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (m¹1), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ω). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. In some embodiments, an mRNA of the disclosure is modified wherein at least 95%, at least 96%, at least 97%, at least 98%, at least 99% or 100% of a specified nucleotide or nucleobase is modified. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above. In some embodiments, an mRNA of the disclosure is uniformly modified with 1-methyl pseudouridine (m¹ψ), meaning that all uridine residues in the mRNA sequence are replaced with 1-methyl pseudouridine (m¹ψ). In some embodiments, at least 95%, at least 96%, at least 97%, at least 98%, at least 99% or 100% of uridines are 1-methyl pseudouridine (m¹ψ).

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mRNAs of the disclosure can include a combination of modifications to the sugar, the nucleobase, and/or the internucleoside linkage. These combinations can include any one or more modifications described herein.

Examples of modified nucleosides and modified nucleoside combinations are provided below in Table 3 and Table 4. These combinations of modified nucleotides can be used to form the mmRNAs of the disclosure. In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

TABLE 3
Combinations of Nucleoside Modifications

| Modified | |
|---|---|
| Nucleotide | Modified Nucleotide Combination |
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2- |
| | thio-uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 4
Modified Nucleosides and Combinations Thereof

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75% UTP
25% 5-Aminoallyl-CTP + 75% CTP/75% 5-Methoxy-UTP + 25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP + 75%
UTP
25% 5-Hydroxymethyl-CTP + 75% CTP/75% 5-Methoxy-UTP + 25%
UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75% 1-Methyl-
pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50% 1-Methyl-
pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25% 1-Methyl-
pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP + 75%
UTP
25% 5-Trifluoromethyl-CTP + 75% CTP/75% 5-Methoxy-UTP + 25%
UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75% 1-Methyl-
pseudo-UTP
50% 5-Methyl-CTP + 50% CTP/25% 5-Methoxy-UTP + 75% UTP
50% 5-Methyl-CTP + 50% CTP/50% 5-Methoxy-UTP + 50% 1-Methyl-
pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25% 1-Methyl-
pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy- methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro- methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75% UTP
75% 5-Aminoallyl-CTP + 25% CTP/75% 5-Methoxy-UTP + 25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP + 75%
UTP
75% 5-Hydroxymethyl-CTP + 25% CTP/75% 5-Methoxy-UTP + 25%
UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75% 1-Methyl-
pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50% 1-Methyl-
pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25% 1-Methyl-
pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP + 75%
UTP
75% 5-Trifluoromethyl-CTP + 25% CTP/75% 5-Methoxy-UTP + 25%
UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the disclosure, polynucleotides of the disclosure may be synthesized to comprise the combinations or single modifications of Table 3 or Table 4.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Functional RNA Elements

In some embodiments, the disclosure provides polynucleotides comprising a modification (e.g., an RNA element), wherein the modification provides a desired translational regulatory activity. Such modifications are described in PCT Application No. PCT/US2018/033519, herein incorporated by reference in its entirety.

In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some embodiments, the disclosure provides an mRNA having one or more structural modifications that inhibits leaky scanning and/or promotes the translational fidelity of mRNA translation, wherein at least one of the structural modifications is a GC-rich RNA element. In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In some embodiments, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In some embodiments, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In some embodiments, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4 (SEQ ID NO: 23). In some embodiments, the sequence comprises a repeating GC-motif [CCG]n, wherein n=5 (SEQ ID NO: 24).

In some embodiments, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In some embodiments, the disclosure provides an mRNA comprising at least one modification, wherein at least one modification is a GC-rich RNA element comprising the sequence set forth in SEQ ID NO: 25, or derivatives or analogs thereof, preceding a Kozak consensus sequence in the 5′ UTR of the mRNA. In some embodiments, the GC-rich element comprises the sequence as set forth in SEQ ID NO: 25 located immediately adjacent to and upstream of the Kozak consensus sequence in the 5′ UTR of the mRNA. In some embodiments, the GC-rich element comprises the sequence as set forth in SEQ ID NO: 25 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth in SEQ ID NO: 25 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence as set forth SEQ ID NO: 26, or derivatives or analogs thereof, preceding a Kozak consensus sequence in the 5′ UTR of the mRNA. In some embodiments, the GC-rich element comprises the sequence as set forth SEQ ID NO: 26 located immediately adjacent to and upstream of the Kozak consensus sequence in the 5′ UTR of the mRNA. In some embodiments, the GC-rich element comprises the sequence as set forth SEQ ID NO: 26 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth SEQ ID NO: 26 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence as set forth in SEQ ID NO: 27, or derivatives or analogs thereof, preceding a Kozak consensus sequence in the 5′ UTR of the mRNA. In some embodiments, the GC-rich element comprises the sequence as set forth in SEQ ID NO: 27 located immediately adjacent to and upstream of the Kozak consensus sequence in the 5′ UTR of the mRNA. In some embodiments, the GC-rich element comprises the sequence as set forth in SEQ ID NO: 27 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth in SEQ ID NO: 27 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence set forth in SEQ ID NO: 25, or derivatives or analogs thereof, preceding a Kozak consensus sequence in the 5′ UTR of the mRNA, wherein the 5′ UTR comprises the sequence set forth in SEQ ID NO: 28.

In some embodiments, the GC-rich element comprises the sequence set forth in SEQ ID NO: 25 located immediately adjacent to and upstream of the Kozak consensus sequence in a 5′ UTR sequence described herein. In some embodiments, the GC-rich element comprises the sequence set forth in SEQ ID NO: 25 located 1, 2, 3, 4, 5, 6, 7, 8, 9 or 10 bases upstream of the Kozak consensus sequence in the 5′ UTR of the mRNA, wherein the 5′ UTR comprises the sequence shown in SEQ ID NO: 28.

In other embodiments, the GC-rich element comprises the sequence set forth in SEQ ID NO: 25 located 1-3, 3-5, 5-7, 7-9, 9-12, or 12-15 bases upstream of the Kozak consensus sequence in the 5′ UTR of the mRNA, wherein the 5′ UTR comprises the sequence set forth in SEQ ID NO: 28.

In some embodiments, the 5′ UTR comprises the sequence set forth in SEQ ID NO: 29.

In some embodiments, the 5′ UTR comprises the sequence set forth in SEQ ID NO: 30.

In some embodiments, the disclosure provides an mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream of the Kozak consensus sequence. In another embodiment, the stable RNA secondary structure is located about 30, about 25, about 20, about 15, about 10, or about 5 nucleotides upstream of the Kozak consensus sequence. In another embodiment, the stable RNA secondary structure is located about 20, about 15, about 10 or about 5 nucleotides upstream of the Kozak consensus sequence. In another embodiment, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to ˜10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these position would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of a the PIC or ribosome at a discrete position or location along an polynucleotide comprising any one or more of the RNA elements described herein is determined by ribosome profiling.

### Delivery Agents

a. Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:

(a) an mRNA comprising a nucleotide sequence encoding OX40L; and

(b) a delivery agent.

Lipid Nanoparticle Formulations

In some embodiments, nucleic acids of the invention (e.g. mRNA) are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g. mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

Ionizable Lipids

In some aspects, the ionizable lipids of the present disclosure may be one or more of compounds of Formula (I):

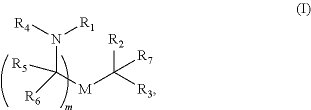

or their N-oxides, or salts or isomers thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R2 and R3, together with the atom to which they are attached, form a heterocycle or carbocycle;

R₄ is selected from the group consisting of hydrogen, a C₃₋₆ carbocycle, —(CH₂)ₙQ, —(CH₂)ₙCHQR,

—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected

from —C(O)O—, —OC(O)—, —OC(O)-M″-C(O)O—, —C(O)N(R′)—,

—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO2, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, C₁, Br, and I; and

m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

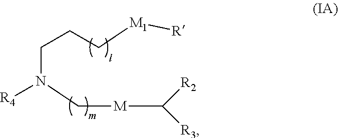

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is

OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

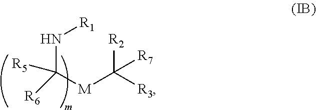

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is
OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (II):

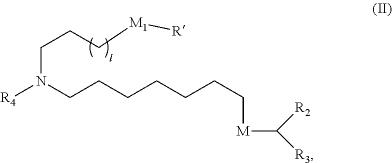

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (I) are of Formula (IIa),

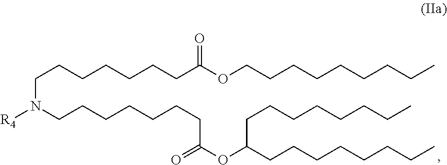

or their N-oxides, or salts or isomers thereof, wherein R₄ is as described herein.

In another embodiment, the compounds of Formula (I) are of Formula (IIb),

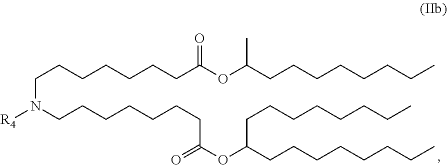

or their N-oxides, or salts or isomers thereof, wherein R₄ is as described herein.

In another embodiment, the compounds of Formula (I) are of Formula (IIc) or (He):

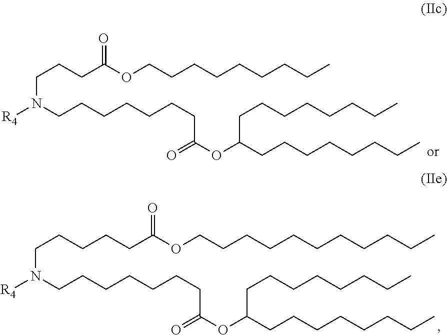

or their N-oxides, or salts or isomers thereof, wherein R₄ is as described herein.

In another embodiment, the compounds of Formula (I) are of Formula (IIf):

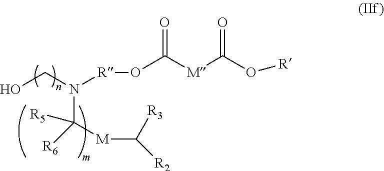

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

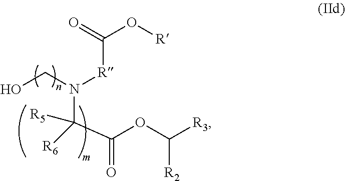

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (I) are of Formula (IIg),

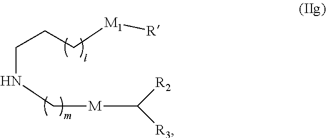

or their N-oxides, or salts or isomers thereof, wherein l is selected from 1, 2, 3, 4, and 5; m is selected from 5, 6, 7, 8, and 9; M₁ is a bond or M′; M and M′ are independently selected from

—C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the ionizable lipids are selected from Compounds 1-280 described in U.S. Application No. 62/475,166.

In some embodiments, the ionizable lipid is

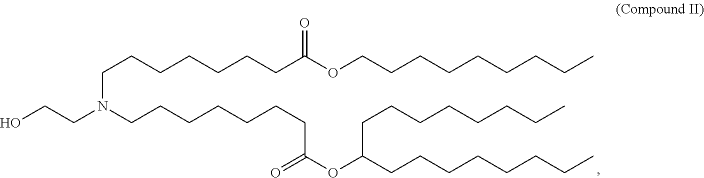 [SMILES: CCCCCCCCCOC(=O)CCCCCCCN(CCO)CCCCCCCC(=O)OC(CCCCCCCC)CCCCCCCC]

or a salt thereof.

In some embodiments, the ionizable lipid is

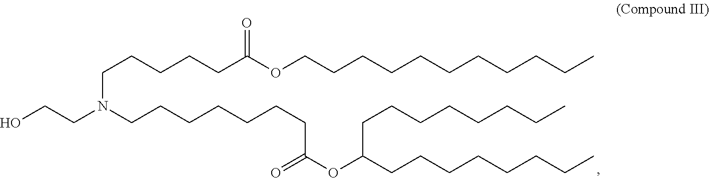 [SMILES: CCCCCCCCCCCOC(=O)CCCCCN(CCO)CCCCCCCC(=O)OC(CCCCCCCC)CCCCCCCC]

or a salt thereof.

In some embodiments, the ionizable lipid is

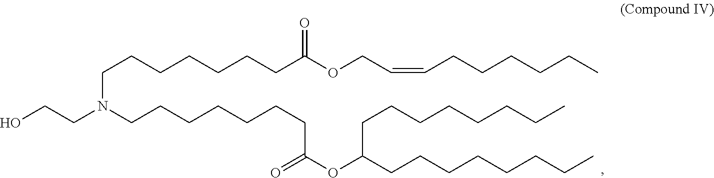 [SMILES: CCCCCC/C=C\COC(=O)CCCCCCCN(CCO)CCCCCCCC(=O)OC(CCCCCCCC)CCCCCCCC]

or a salt thereof.

In some embodiments, the ionizable lipid is

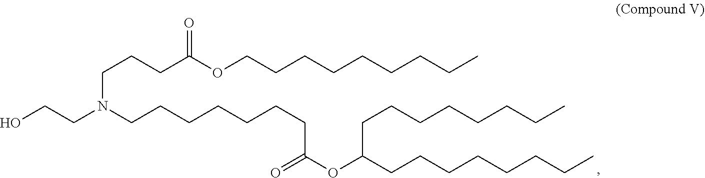 [SMILES: CCCCCCCCCOC(=O)CCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such lipids may be referred to as cationic or ionizable (amino) lipids. Lipids may also be zwitterionic, i.e., neutral molecules having both a positive and a negative charge.

In some aspects, the ionizable lipids of the present disclosure may be one or more of compounds of formula (III),

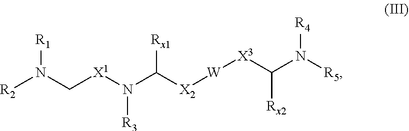

or salts or isomers thereof, wherein

W is

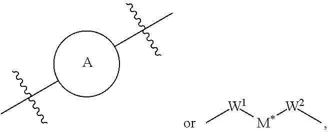

ring A is

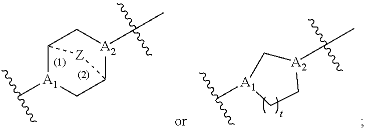

t is 1 or 2;

A₁ and A2 are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting

of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

when ring A is

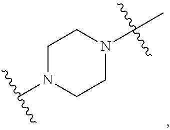 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (IIIa1)-(IIIa8):

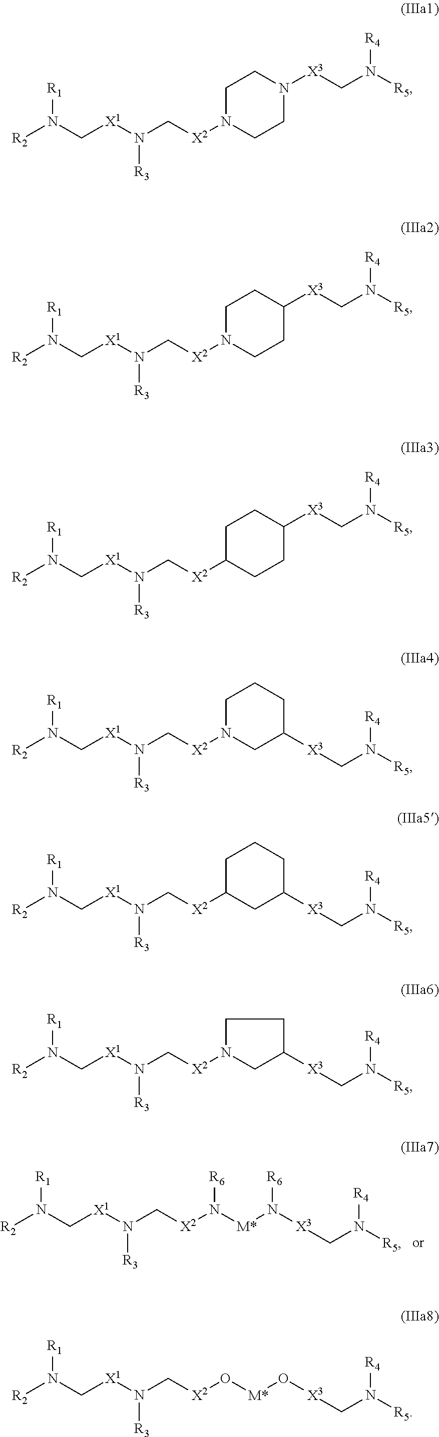

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

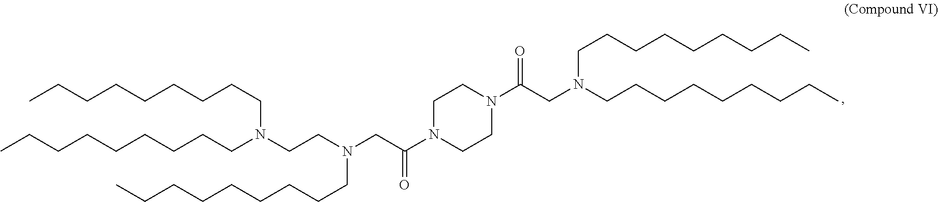 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

In some embodiments, the ionizable lipid is (Compound VII), or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

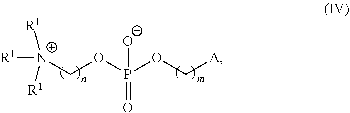

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

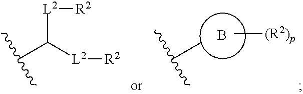

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

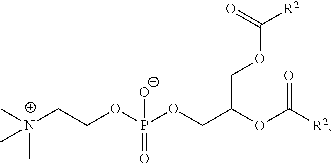

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

(i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

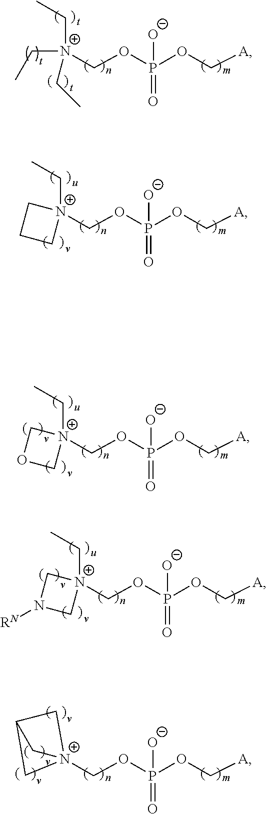

or a salt thereof, wherein:

each t is independently 1, 2, 3, 4, 5, 6, 7, 8, 9, or 10;

each u is independently 0, 1, 2, 3, 4, 5, 6, 7, 8, 9, or 10; and

each v is independently 1, 2, or 3.

In certain embodiments, a compound of Formula (IV) is of Formula (IV-a):

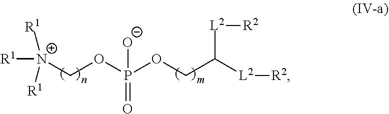

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

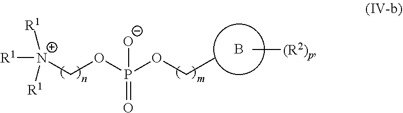

or a salt thereof.

(ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O.

In certain embodiments, the compound of Formula (IV) is of Formula (IV-c):

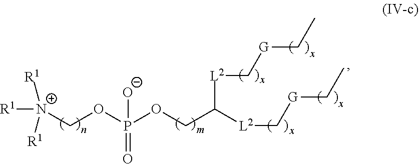

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following formulae:

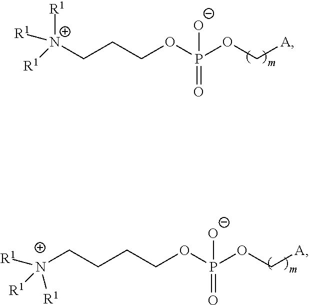

or a salt thereof.

Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following:

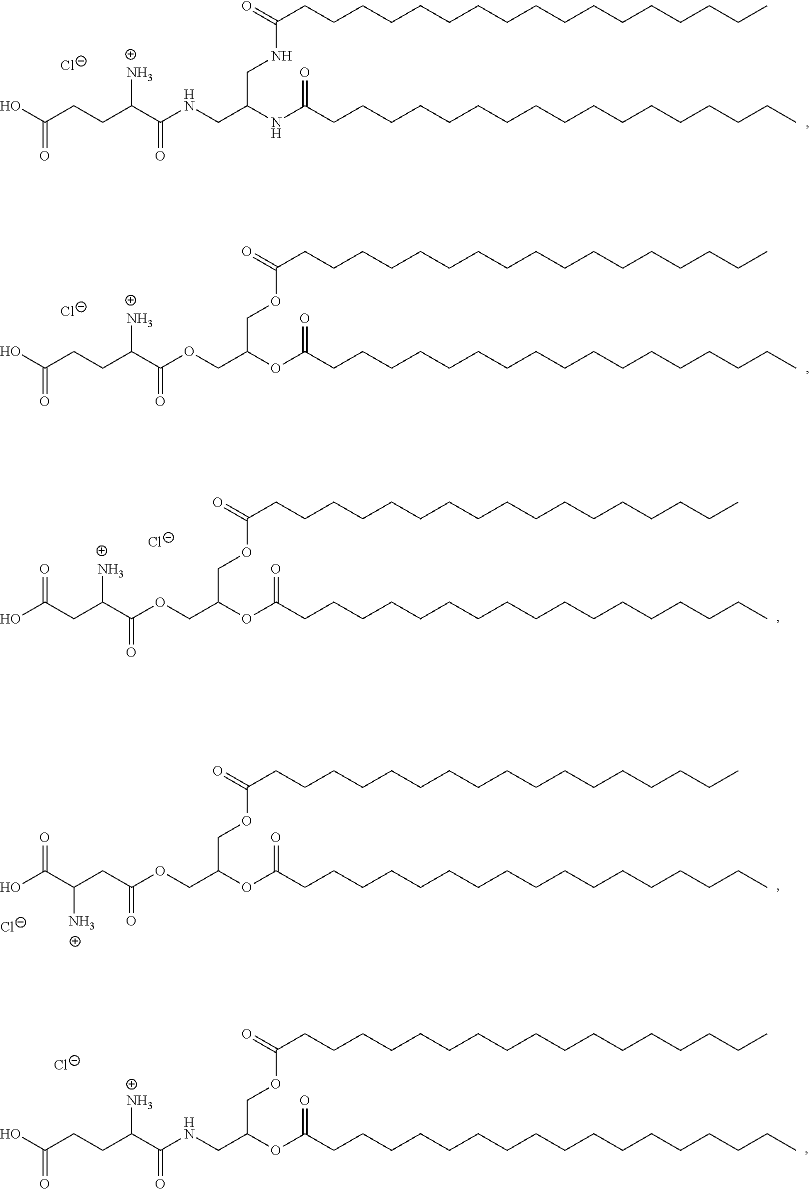

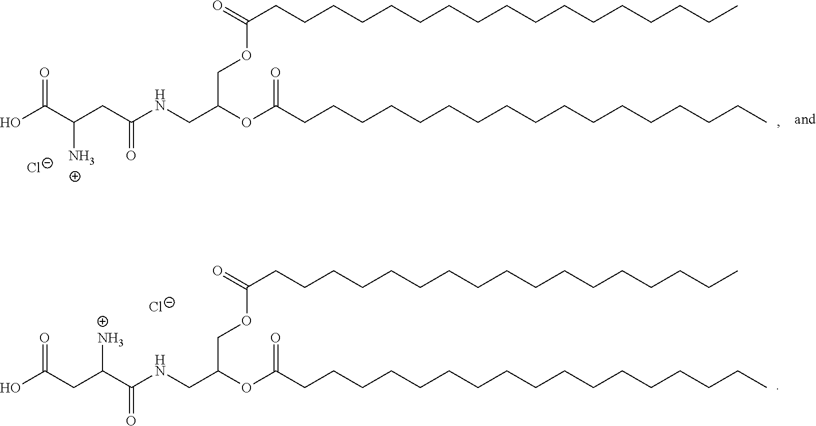 [SMILES: CCCCCCCCCCCCCCCCCC(=O)OCC(CNC(=O)CC([NH3+])C(=O)O)OC(=O)CCCCCCCCCCCCCCCCC ; [Cl-] ; CCCCCCCCCCCCCCCCCC(=O)OCC(CNC(=O)C([NH3+])CC(=O)O)OC(=O)CCCCCCCCCCCCCCCCC ; [Cl-]]

Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DS G), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

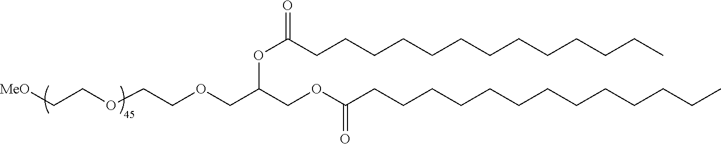

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

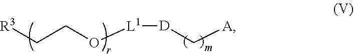

or salts thereof, wherein:

R³ is —OR^{O};

R^{O} is hydrogen, optionally substituted alkyl, or an oxygen protecting group;
r is an integer between 1 and 100, inclusive; L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

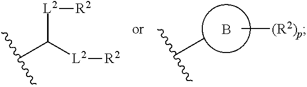

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

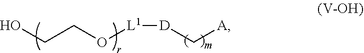

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

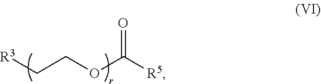

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (VI) is of Formula (VI—OH):

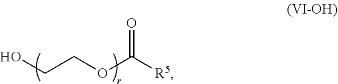

or a salt thereof. In some embodiments, r is 45.

In yet other embodiments the compound of Formula (VI) is:

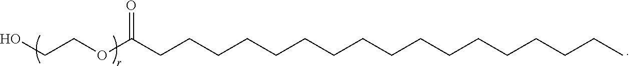

or a salt thereof.

In one embodiment, the compound of Formula (VI) is

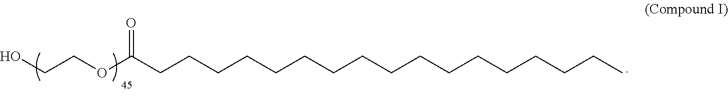

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the invention comprises an ionizable cationic lipid of any of Formula I, II or III, a phospholipid comprising DSPC, a structural lipid, and a PEG lipid comprising PEG-DMG.

In some embodiments, a LNP of the invention comprises an ionizable cationic lipid of any of Formula I, II or III, a phospholipid comprising DSPC, a structural lipid, and a PEG lipid comprising a compound having Formula VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid having Formula IV, a structural lipid, and a PEG lipid comprising a compound having Formula VI.

In some embodiments, a LNP of the invention comprises an ionizable cationic lipid of

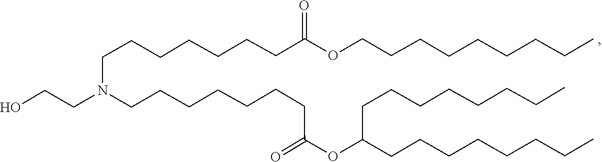 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP of the invention comprises an ionizable cationic lipid of

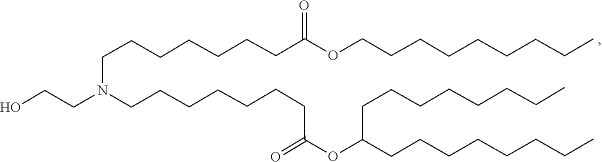 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the invention comprises an ionizable cationic lipid of

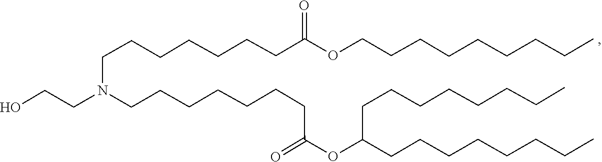 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP of the invention comprises an ionizable cationic lipid of

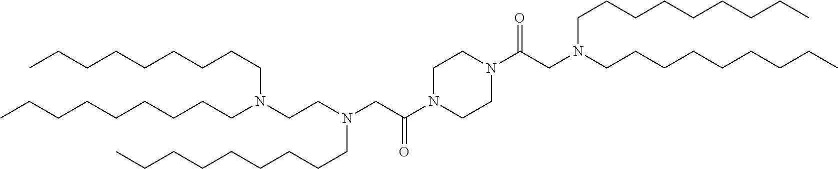 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an ionizable cationic lipid of a phospholipid comprising DOPE, a structural lipid comprising cholesterol, and a PEG lipid comprising a compound having Formula VII.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1.

In some embodiments, a LNP of the invention comprises a wt/wt ratio of the ionizable cationic lipid component to the RNA of from about 10:1 to about 100:1.

In some embodiments, a LNP of the invention comprises a wt/wt ratio of the ionizable cationic lipid component to the RNA of about 20:1.

In some embodiments, a LNP of the invention comprises a wt/wt ratio of the ionizable cationic lipid component to the RNA of about 10:1.

In some embodiments, a LNP of the invention has a mean diameter from about 50 nm to about 150 nm.

In some embodiments, a LNP of the invention has a mean diameter from about 70 nm to about 120 nm.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1 14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C2-14 alkenyl” means an optionally substituted linear or branched hydrocarbon including 2 14 carbon atoms and at least one carbon-carbon double bond. An alkenyl group may include one, two, three, four, or more carbon-carbon double bonds. For example, C18 alkenyl may include one or more double bonds. A C18 alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C2-14 alkynyl” means an optionally substituted linear or branched hydrocarbon including 2 14 carbon atoms and at least one carbon-carbon triple bond. An alkynyl group may include one, two, three, four, or more carbon-carbon triple bonds. For example, C18 alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C3-6 carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)2-, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)2R″″, in which each OR are alkoxy groups that can be the same or different and R″″ is an alkyl or alkenyl group), a phosphate (e.g., P(O)43−), a thiol (e.g., SH), a sulfoxide (e.g., S(O)R), a sulfinic acid (e.g., S(O)OH), a sulfonic acid (e.g., S(O)2OH), a thial (e.g., C(S)H), a sulfate (e.g., S(O)42−), a sulfonyl (e.g., S(O)2), an amide (e.g., C(O)NR2, or N(R)C(O)R), an azido (e.g., N3), a nitro (e.g., NO2), a cyano (e.g., CN), an isocyano (e.g., NC), an acyloxy (e.g., OC(O)R), an amino (e.g., NR2, NRH, or NH2), a carbamoyl (e.g., OC(O)NR2, OC(O)NRH, or OC(O)NH2), a sulfonamide (e.g., S(O)2NR2, S(O)2NRH, S(O)2NH2, N(R)S(O)2R, N(H)S(O)2R, N(R)S(O)2H, or N(H)S(O)2H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C1 6 alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N□O or N+—O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C1-C6 alkyl, C1-C6 alkenyl, C1-C6 alkynyl, 3-14-membered carbocycle or 3-14-membered heterocycle) derivatives.

Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

Nanoparticle Compositions

In some embodiments, the pharmaceutical compositions disclosed herein are formulated as lipid nanoparticles (LNP). Accordingly, the present disclosure also provides nanoparticle compositions comprising (i) a lipid composition comprising a delivery agent such as compound as described herein, and (ii) at least one mRNA encoding OX40L. In such nanoparticle composition, the lipid composition disclosed herein can encapsulate the at least one mRNA encoding OX40L.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid: about 5-25% structural lipid: about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group. In one embodiment, the ionizable lipid may be selected from, but not limited to, a ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide is formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

Other Delivery Agents

a. Liposomes, Lipoplexes, and Lipid Nanoparticles

In some embodiments, the compositions or formulations of the present disclosure comprise a delivery agent, e.g., a liposome, a lioplexes, a lipid nanoparticle, or any combination thereof. The polynucleotides described herein (e.g., a polynucleotide comprising a nucleotide sequence encoding a polypeptide) can be formulated using one or more liposomes, lipoplexes, or lipid nanoparticles. Liposomes, lipoplexes, or lipid nanoparticles can be used to improve the efficacy of the mRNAs directed protein production as these formulations can increase cell transfection by the mRNA; and/or increase the translation of encoded protein. The liposomes, lipoplexes, or lipid nanoparticles can also be used to increase the stability of the mRNAs.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the mRNAs described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the mRNAs described herein can be formulated in a cationic oil-in-water emulsion where the emulsion particle comprises an oil core and a cationic lipid that can interact with the mRNA anchoring the molecule to the emulsion particle. In some embodiments, the mRNAs described herein can be formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the mRNAs described herein can be formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the mRNAs described herein can be formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)-N,N-dimethyl-3-nonyldocosa-13,16-dien-1-amine (L608), (20Z,23Z)-N,N-dimethylnonacosa-20,23-dien-10-amine, (17Z,20Z)-N,N-dimemylhexacosa-17,20-dien-9-amine, (16Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyleptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)-N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, mRNAs, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the mRNA described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the mRNAs described herein are formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the mRNAs described herein can be formulated for controlled release and/or targeted delivery. As used herein, “controlled release” refers to a pharmaceutical composition or compound release profile that conforms to a particular pattern of release to effect a therapeutic outcome. In one embodiment, the mRNAs can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the mRNAs described herein can be encapsulated in a therapeutic nanoparticle, referred to herein as “therapeutic nanoparticle mRNAs.” Therapeutic nanoparticles can be formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle mRNA can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the mRNAs described herein can be formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle mRNA can be formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the mRNAs described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the mRNAs can be formulated in lipid nanoparticles using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the mRNAs described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the mRNAs can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the mRNAs described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

b. Lipidoids

In some embodiments, the compositions or formulations of the present disclosure comprise a delivery agent, e.g., a lipidoid. The mRNAs described herein (e.g., an mRNA comprising a nucleotide sequence encoding a polypeptide) can be formulated with lipidoids. Complexes, micelles, liposomes or particles can be prepared containing these lipidoids and therefore to achieve an effective delivery of the mRNA, as judged by the production of an encoded protein, following the injection of a lipidoid formulation via localized and/or systemic routes of administration. Lipidoid complexes of mRNAs can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C12-200 (including derivatives and variants), and MD1, can be tested for in vivo activity. The lipidoid “98N12-5” is disclosed by Akinc et al., Mol Ther. 2009 17:872-879. The lipidoid “C12-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the mRNAs described herein can be formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to mRNAs. Lipidoids and mRNA formulations comprising lipidoids are described in Intl. Pub. No. WO 2015051214 (herein incorporated by reference in its entirety.

### Pharmaceutical Compositions

The present disclosure includes pharmaceutical compositions comprising an OX40L encoding mRNA or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of an mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of mRNA. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

The present disclosure also includes kits comprising a container comprising a mRNA encoding a polypeptide that enhances an immune response. In another embodiment, the kit comprises a container comprising a mRNA encoding a polypeptide that enhances an immune response, as well as one or more additional mRNAs encoding one or more antigens or interest. In other embodiments, the kit comprises a first container comprising the mRNA encoding a polypeptide that enhances an immune response and a second container comprising one or more mRNAs encoding one or more antigens of interest. In particular embodiments, the mRNAs for enhancing an immune response and the mRNA(s) encoding an antigen(s) are present in the same or different nanoparticles and/or pharmaceutical compositions. In particular embodiments, the mRNAs are lyophilized, dried, or freeze-dried.

### Kits

In some embodiments, the disclosure provides a kit comprising an OX40L encoding mRNA, or composition (e.g. lipid nanoparticle) comprising an OX40L encoding mRNA, as described herein. In some embodiments, a kit comprises a container comprising a pharmaceutical composition comprising a lipid nanoparticle comprising an mRNA encoding a human OX40L polypeptide; and a pharmaceutically acceptable carrier, wherein the pharmaceutical composition comprises 2 mg/ml of the mRNA, and a package insert comprising instructions for administration of the mRNA by intratumoral injection to treat or delay progression of ovarian cancer, or other cancers such as solid tumors, lymphomas or epithelial origin cancers (e.g., an epithelial cancer of ovary, fallopian tube or peritoneum), in a human patient

In some embodiments, a kit comprises a container comprising a pharmaceutical composition comprising a lipid nanoparticle comprising an mRNA encoding a human OX40L polypeptide; and a pharmaceutically acceptable carrier, wherein the pharmaceutical composition comprises 2 mg/ml of the mRNA, and a package insert comprising instructions for administration of the mRNA by intratumoral injection and instruction for use in combination with a second composition comprising a PD-1 antagonist, a PD-L1 antagonist or a CTLA-4 antagonist, for use in treating or delaying progression of ovarian cancer, or other cancers such as solid tumors, lymphomas or epithelial origin cancers (e.g., an epithelial cancer of ovary, fallopian tube or peritoneum), in a human patient.

In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition. In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition for treating or delaying progression of an ovarian cancer, or other cancers such as solid tumors, lymphomas or epithelial origin cancers (e.g., an epithelial cancer of ovary, fallopian tube or peritoneum), in an individual. In some aspects, the package insert further comprises instructions for administration of the lipid nanoparticle or pharmaceutical composition in combination with a composition comprising a checkpoint inhibitor polypeptide and an optional pharmaceutically acceptable carrier for treating or delaying progression of an ovarian cancer, or other cancers such as solid tumors, lymphomas or epithelial origin cancers (e.g., an epithelial cancer of ovary, fallopian tube or peritoneum), in an individual.

In some embodiments, a kit comprises a medicament comprising a lipid nanoparticle encapsulating the an OX40L encoding mRNA described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier. In some embodiments, a kit comprises a medicament comprising a lipid nanoparticle encapsulating an OX40L encoding mRNA described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier for treating or delaying progression of ovarian cancer in an individual. In some aspects, the kit further comprises a package insert comprising instructions for administration of the first medicament prior to, current with, or subsequent to administration of the second medicament for treating or delaying progression of ovarian cancer, or other cancers such as solid tumors, lymphomas or epithelial origin cancers (e.g., an epithelial cancer of ovary, fallopian tube or peritoneum), in an individual.

In some embodiments, the kit comprises a lipid nanoparticle comprising an mRNA encoding a human OX40L polypeptide and a PD-L1 antagonist. In some embodiments, the PD-L1 antagonist is selected from the group consisting of durvalumab, avelumab, and atezolizumab. In some embodiments, the PD-L1 antagonist is durvalumab. In some embodiments, the instructions provide for administration of mRNA at a dose of 1.0-8.0 mg. In some embodiments, the instructions provide for administration of mRNA at a dose of 8.0 mg. In some embodiments, the instructions provide for administration of the PD-L1 antagonist (e.g., durvalumab) at a dose of 1500 mg.

### Definitions

Abscopal effect: As used herein, “abscopal effect” refers to a phenomenon in the treatment of cancer, including metastatic cancer, where localized administration of a treatment (e.g., mRNA encoding OX40L) to a tumor causes not only a reduction in size of the treated tumor but also a reduction in size of tumors outside the treated area. In some embodiments, the abscopal effect is a local, regional abscopal effect, wherein a proximal or nearby tumor relative to the treated tumor is affected. In some embodiments, the absocpal effect occurs in a distal tumor relative to the treated tumor. In some embodiments, treatment (e.g., mRNA encoding OX40L) is administered via intratumoral injection, resulting in a reduction in tumor size of the injected tumor and a proximal or distal uninjected tumor.

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, intradermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Cleavable Linker: As used herein, the term “cleavable linker” refers to a linker, typically a peptide linker (e.g., about 5-30 amino acids in length, typically about 10-20 amino acids in length) that can be incorporated into multicistronic mRNA constructs such that equimolar levels of multiple genes can be produced from the same mRNA. Non-limiting examples of cleavable linkers include the 2A family of peptides, including F2A, P2A, T2A and E2A, first discovered in picornaviruses, that when incorporated into an mRNA construct (e.g., between two polypeptide domains) function by making the ribosome skip the synthesis of a peptide bond at C-terminus of the 2A element, thereby leading to separation between the end of the 2A sequence and the next peptide downstream.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Dosing interval: As used herein, the term “dosing interval”, “dosage interval” or “dosing regimen” refers to a discrete amount of time, expressed in units of time, (e.g., 14 days) that transpires between individual administrations (plural) of a dose of a therapeutic composition (e.g., a composition comprising an mRNA). For example, in some embodiments, a dosing interval starts on the day a first dose is administered (e.g., initial dose), and ends on the day a second dose (e.g., a subsequent dose) is administered. In some embodiments, there are multiple dosing intervals during treatment.

Encapsulate: As used herein, the term “encapsulate” means to enclose, surround, or encase. In some embodiments, a compound, an mRNA, or other composition may be fully encapsulated, partially encapsulated, or substantially encapsulated. For example, in some embodiments, an mRNA of the disclosure may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agent or prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Epithelial origin cancer: As used herein, the phrase “epithelial origin cancer” is intended to encompass cancers originating from the epithelial tissue, such as of the ovary or other tissue in the vicinity of the ovary including the fallopian tube and/or the peritoneum.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Fragment: A “fragment,” as used herein, refers to a portion. For example, fragments of proteins may include polypeptides obtained by digesting full-length protein isolated from cultured cells or obtained through recombinant DNA techniques.

Heterologous: As used herein, “heterologous” indicates that a sequence (e.g., an amino acid sequence or the nucleic acid that encodes an amino acid sequence) is not normally present in a given polypeptide or nucleic acid. For example, an amino acid sequence that corresponds to a domain or motif of one protein may be heterologous to a second protein.

Hydrophobic amino acid: As used herein, a “hydrophobic amino acid” is an amino acid having an uncharged, nonpolar side chain. Examples of naturally occurring hydrophobic amino acids are alanine (Ala), valine (Val), leucine (Leu), isoleucine (Ile), proline (Pro), phenylalanine (Phe), methionine (Met), and tryptophan (Trp).

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two mRNA sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387, 1984, BLASTP, BLASTN, and FASTA, Altschul, S. F. et al., J. Molec. Biol., 215, 403, 1990.

Immune checkpoint inhibitor: An “immune checkpoint inhibitor” or simply “checkpoint inhibitor” refers to a molecule that prevents immune cells from being turned off by cancer cells. As used herein, the term checkpoint inhibitor refers to polypeptides (e.g., antibodies) or polynucleotides encoding such polypeptides (e.g., mRNAs) that neutralize or inhibit inhibitory checkpoint molecules such as cytotoxic T-lymphocyte-associated protein 4 (CTLA-4), programmed death 1 receptor (PD-1), or PD-1 ligand 1 (PD-L1).

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In some cases, the administration of a nanoparticle comprising a lipid component and an encapsulated therapeutic agent can trigger an immune response, which can be caused by (i) the encapsulated therapeutic agent (e.g., an mRNA), (ii) the expression product of such encapsulated therapeutic agent (e.g., a polypeptide encoded by the mRNA), (iii) the lipid component of the nanoparticle, or (iv) a combination thereof.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule. For example, an amino acid sequence of a heterologous polypeptide (e.g., a BH3 domain) may be inserted into a scaffold polypeptide (e.g. a SteA scaffold polypeptide) at a site that is amenable to insertion. In some embodiments, an insertion may be a replacement, for example, if an amino acid sequence that forms a loop of a scaffold polypeptide (e.g., loop 1 or loop 2 of SteA or a SteA derivative) is replaced by an amino acid sequence of a heterologous polypeptide.

Insertion Site: As used herein, an “insertion site” is a position or region of a scaffold polypeptide that is amenable to insertion of an amino acid sequence of a heterologous polypeptide. It is to be understood that an insertion site also may refer to the position or region of the mRNA that encodes the polypeptide (e.g., a codon of an mRNA that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Linker: As used herein, a “linker” (including a subunit linker, and a heterologous polypeptide linker as referred to herein) refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Lymphoma: As used herein, the term “lymphoma” refers to a malignancy that originates in a lymphocyte, including malignancies that originate in a B lymphocyte and malignancies that originate in a T lymphocyte.

Metastasis: As used herein, the term “metastasis” means the process by which cancer spreads from the place at which it first arose as a primary tumor to distant locations in the body. A secondary tumor that arose as a result of this process may be referred to as “a metastasis.”

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

microRNA (miRNA): As used herein, a “microRNA (miRNA)” is a small non-coding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22 nucleotides long.

microRNA-122 (miR-122): As used herein, “microRNA-122 (miR-122)” refers to any native miR-122 from any vertebrate source, including, for example, humans, unless otherwise indicated. miR-122 is typically highly expressed in the liver, where it may regulate fatty-acid metabolism. miR-122 levels are reduced in liver cancer, for example, hepatocellular carcinoma. miR-122 is one of the most highly-expressed miRNAs in the liver, where it regulates targets including but not limited to CAT-1, CD320, AldoA, Hjv, Hfe, ADAM10, IGFR1, CCNG1, and ADAM17. Mature human miR-122 may have a sequence of AACGCCAUUAUCACACUAAAUA (SEQ ID NO: 13, corresponding to hsa-miR-122-3p) or UGGAGUGUGACAAUGGUGUUUG (SEQ ID NO: 19, corresponding to hsa-miR-122-5p).

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of an mRNA to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70420 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Operably linked: As used herein, the phrase “operably linked” refers to a functional connection between two or more molecules, constructs, transcripts, entities, moieties or the like.

Ovarian cancer: As used herein, the phrase “ovarian cancer” refers to cancers originating in any ovarian tissue, including tumors of ovarian epithelial origin, ovarian stromal origin and ovarian germ cell origin and at any stage, including stage I, IA, IB, IC, II, IIA, IIB, IIIA1, IIIA2, IIIB, IIIC, IVA and IVB ovarian cancers.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a human patient having ovarian cancer.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as an mRNA) into a cell.

Treating: As used herein, the term “treating” refers to partially or completely alleviating, ameliorating, improving, relieving, delaying onset of, inhibiting progression of, reducing severity of, and/or reducing incidence of one or more symptoms or features of ovarian cancer. For example, “treating” cancer may refer to inhibiting survival, growth, and/or spread of a tumor. Treatment may be measured by reduction in numbers of tumors or reduction in size of a particular tumor and/or reduction in metastasis. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### OTHER EMBODIMENTS

The disclosure relates to the following embodiments. Throughout this section, the term embodiment is abbreviated as ‘E’ followed by an ordinal. For example, E1 is equivalent to Embodiment 1.

E1. A method for treating ovarian cancer, or a solid tumor, lymphoma or epithelial origin cancer, in a human patient by inducing or enhancing an anti-tumor immune response, comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating ovarian cancer, or a solid tumor, lymphoma or epithelial origin cancer, in the patient by inducing or enhancing an anti-tumor immune response.
E2. The method of embodiment 1, wherein treatment results in a reduction in tumor size or inhibition in tumor growth in the injected tumor in the patient.
E3. The method of any one of embodiment 1 or 2, wherein treatment results in a reduction in size or inhibition of growth in an uninjected tumor in the patient.
E4. The method of embodiment 3, wherein the uninjected tumor is at a location proximal to the injected tumor in the patient.
E5. The method of embodiment 3, wherein the uninjected tumor is at a location distal to the injected tumor in the patient.
E6. The method of any one of embodiments 3-5, wherein treatment results in a reduction in size or inhibition of growth of an uninjected tumor through an abscopal effect in the patient.
E7. The method of any one of the preceding embodiments, wherein treatment results in increased expression of human OX40L polypeptide in the tumor.
E8. The method of any one of the preceding embodiments, wherein treatment results in increased expression of human OX40L polypeptide in immune cells in the tumor microenvironment.
E9. The method of any one of the preceding embodiments, wherein the anti-tumor immune response in the patient comprises T cell activation, T cell proliferation, and/or T cell expansion.
E10. The method of embodiment 9, wherein the T cells are CD4+ T cells.
E11. The method of embodiment 9, wherein the T cells are CD8+ T cells.
E12. The method of embodiment 9, wherein the T cells are CD4+ T cells and CD8+ T cells.
E13. The method of any one of embodiments 9-12, wherein the anti-tumor immune response results in a reduction in size or inhibition of growth of the injected tumor.
E14. The method of any one of embodiments 9-12, wherein the anti-tumor immune response results in a reduction in size or inhibition of growth of an uninjected tumor through an abscopal effect in the patient.
E15. The method of any one of the preceding embodiments, wherein the patient is administered a dose of mRNA selected from 1.0-8.0 mg, 1.0-6.0 mg, 1.0-4.0 mg, and 1.0-2.0 mg of mRNA.
E16. The method of any one of the preceding embodiments, wherein the mRNA is administered in a dosing regimen selected from 7 to 28 days, 7 to 21 days, 7 to 14 days, 28 days, 21 days, 14 days and 7 days.
E17. The method of any one of embodiments 1-15, wherein the mRNA is administered every 2 weeks in a 28-day cycle.
E18. The method of embodiment 16 or 17, wherein the mRNA is administered at a dose of 8.0 mg.
E19. A method for treating ovarian cancer, or a solid tumor, lymphoma or epithelial origin cancer, in a human patient by inducing or enhancing an anti-tumor immune response, comprising administering to the patient by intratumoral injection an effective amount of a pharmaceutical composition comprising: an LNP comprising an mRNA encoding a human OX40L polypeptide; and a pharmaceutically acceptable carrier, wherein the patient is administered a dose of 1.0-8.0 mg of mRNA in a dosing regimen from 7 to 21 days, thereby treating ovarian cancer, or a solid tumor, lymphoma or epithelial origin cancer, in the patient by inducing or enhancing an anti-tumor immune response.
E20. The method of embodiment 19, wherein the patient is administered a dose of 1.0-6.0 mg mRNA.
E21. The method of embodiment 19, wherein the patient is administered a dose of 1.0-4.0 mg mRNA.
E22. The method of embodiment 19, wherein the patient is administered a dose of 1.0-2.0 mg mRNA.
E23. The method of embodiment 19, wherein the patient is administered a dose of 1.0 mg mRNA.
E24. The method of embodiment 19, wherein the patient is administered a dose of 2.0 mg mRNA.
E25. The method of embodiment 19, wherein the patient is administered a dose of 4.0 mg mRNA.
E26. The method of embodiment 19, wherein the patient is administered a dose of 8.0 mg mRNA.
E27. The method of any one of embodiments 19-26, wherein the dose is administered every 14 days.
E28. The method of any one of embodiments 19-26, wherein the mRNA is administered every 2 weeks in a 28-day cycle.
E29. The method of any one of embodiments 19-26, wherein the mRNA is administered every 2 weeks for 1-6 months.
E30. The method of any one of embodiments 19-26, wherein the mRNA is administered on day 1 and day 15 (±2 days) of a 28-day cycle until the tumor lesion resolves.
E31. The method of any one of embodiments 19-30, wherein treatment results in a reduction in tumor size or inhibition in tumor growth in the injected tumor in the patient.
E32. The method of any one of embodiments 19-31, wherein treatment results in a reduction in size or inhibition of growth in an uninjected tumor in the patient.
E33. The method of embodiment 32, wherein the uninjected tumor is at a location proximal to the injected tumor in the patient.
E34. The method of embodiment 32, wherein the uninjected tumor is at a location distal to the injected tumor in the patient.
E35. The method of any one of embodiments 32-34, wherein treatment results in a reduction in size or inhibition of growth of an uninjected tumor through an abscopal effect in the patient.
E36. The method of any one of embodiments 19-35, wherein treatment results in increased expression of human OX40L polypeptide in the tumor.
E37. The method of any one of embodiments 19-36, wherein treatment results in increased expression of human OX40L polypeptide in immune cells in the tumor microenvironment.
E38. The method of any one of embodiments 19-37, wherein the anti-tumor immune response in the patient comprises T cell activation, T cell proliferation, and/or T cell expansion.
E39. The method of embodiment 38, wherein the T cells are CD4+ T cells.
E40. The method of embodiment 38, wherein the T cells are CD8+ T cells.
E41. The method of embodiment 38, wherein the T cells are CD4+ T cells and CD8+ T cells.
E42. The method of any one of embodiments 35-38, wherein the anti-tumor immune response results in a reduction in size or inhibition of growth of the injected tumor.
E43. The method of any one of embodiments 35-39, wherein the anti-tumor immune response results in a reduction in size or inhibition of growth of an uninjected tumor through an abscopal effect in the patient.
E44. The method of any one of the preceding embodiments, wherein the patient has a superficial tumor lesion amenable to injection.
E45. The method of any one of the preceding embodiments, wherein the patient has a visceral tumor lesion and intratumor injection is facilitated by imaging guidance.
E46. The method of any one of the preceding embodiments, wherein the mRNA is administered by a single injection.
E47. The method of any one embodiments 1-46, wherein the mRNA is administered by multiple injections into one or more different sites within the same tumor lesion or divided across several tumor lesions.
E48. The method of any one of the preceding embodiments, wherein the pharmaceutically acceptable carrier is a solution suitable for intratumoral injection.
E49. The method of embodiment 48, wherein the solution comprises a buffer.
E50. The method of any one of the preceding embodiments, wherein the human OX40L polypeptide comprises the amino acid sequence set forth in SEQ ID NO: 1.
E51. The method of any one of the preceding embodiments, wherein the mRNA comprises an open reading frame comprising a nucleotide sequence at least 90% identical to the nucleotide sequence set forth in SEQ ID NO: 4.
E52. The method of any one of the preceding embodiments, wherein the mRNA comprises an open reading frame comprising the nucleotide sequence set forth in SEQ ID NO: 4.
E53. The method of any one of the preceding embodiments, wherein the mRNA comprises a 3′ untranslated region (UTR) comprising at least one microRNA-122 (miR-122) binding site.
E54. The method of embodiment 53, wherein the miR-122 binding site is a miR-122-3p binding site.
E55. The method of embodiment 53, wherein the miR-122 binding site is a miR-122-5p binding site.
E56. The method of embodiment 55, wherein the miR-122-5p binding site comprises the nucleotide sequence set forth in SEQ ID NO: 20.
E57. The method of embodiment 55, wherein the 3′UTR comprises the nucleotide sequence set forth in SEQ ID NO: 17.
E58. The method of embodiment 52, wherein the mRNA comprises a 5′ untranslated region (UTR) comprising the nucleotide sequence set forth in SEQ ID NO: 15.
E59. The method of embodiment 52, wherein the mRNA comprises a 5′ cap.
E60. The method of embodiment 52, wherein the mRNA comprises a poly-A tail of about 100 nucleotides in length.
E61. The method of any one of embodiments 1-60, wherein the mRNA comprises a nucleotide sequence at least 90% identical to the nucleotide sequence set forth in SEQ ID NO: 5.
E62. The method of any one of embodiments 1-60, wherein the mRNA comprises the nucleotide sequence set forth in SEQ ID NO: 5.
E63. The method of any one of the preceding embodiments, wherein the mRNA is chemically modified.
E64. The method of embodiment 63, wherein the mRNA is fully modified with chemically-modified uridines.
E65. The method of embodiment 64, wherein the chemically-modified uridines are N1-methylpseudouridines (m1ψ).
E66. The method of embodiment 64, wherein the mRNA is fully modified with 5-methylcytosine or is fully modified with N1-methylpseudouridines (m1ψ) and 5-methylcytosine.
E67. The method of any one of the preceding embodiments, wherein the LNP comprises a compound having the formula:

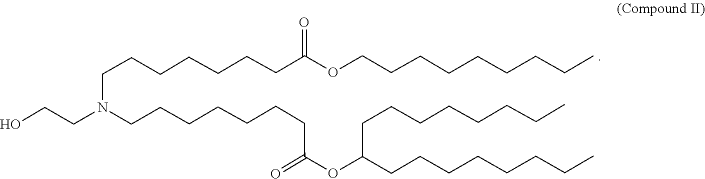 [SMILES: CCCCCCCCCOC(=O)CCCCCCCN(CCO)CCCCCCCC(=O)OC(CCCCCCCC)CCCCCCCC]

E68. The method of embodiment 67, wherein the LNP further comprising a phospholipid, a structural lipid, and a PEG lipid.
E69. The method of any one of embodiments 1-66, wherein the LNP comprises a molar ratio of about 20-60% ionizable amino lipid, about 5-25% phospholipid, about 25-55% structural lipid, and about 0.5-1.5% PEG lipid.
E70. The method of embodiment 69, wherein the LNP comprises a molar ratio of about 50% ionizable amino lipid, about 10% phospholipid, about 38.5% structural lipid, and about 1.5% PEG lipid.
E71. The method of embodiment 69, wherein the LNP comprises a molar ratio of about 50% ionizable amino lipid, about 10% phospholipid, about 38.5% cholesterol, and about 1.5% PEG-DMG.
E72. The method of any one of embodiments 69-71, wherein the ionizable amino lipid comprises a compound having the formula:

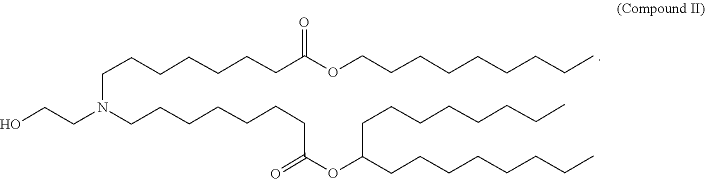 [SMILES: CCCCCCCCCOC(=O)CCCCCCCN(CCO)CCCCCCCC(=O)OC(CCCCCCCC)CCCCCCCC]

E73. The method of any one of the preceding embodiments, further comprising administering an effective amount of a PD-1 antagonist, a PD-L1 antagonist or a CTLA-4 antagonist.
E74. The method of embodiment 73, wherein the PD-1 antagonist is an antibody or antigen binding portion thereof that specifically binds to PD-1.
E75. The method of embodiment 73, wherein the PD-L1 antagonist is an antibody or antigen binding portion thereof that specifically binds to PD-L1.
E76. The method of embodiment 73, wherein the CTLA-4 antagonist is an antibody or antigen binding portion thereof that specifically binds to CTLA-4.
E77. The method of embodiment 74, wherein the PD-1 antagonist is selected from the group consisting of nivolumab, pembrolizumab, and pidilizumab.
E78. The method of embodiment 75, wherein the PD-L1 antagonist is selected from the group consisting of durvalumab, avelumab, and atezolizumab.
E79. The method of embodiment 78, wherein the PD-L1 antagonist is durvalumab.
E80. The method of embodiment 76, wherein the CTLA-4 antagonist is selected from the group consisting of ipilimumab and tremelimumab.
E81. A method for treating ovarian cancer, or a solid tumor, lymphoma or epithelial origin cancer, in a human patient by inducing or enhancing an anti-tumor immune response, comprising administering to the patient (i) by intratumoral injection an effective amount of a pharmaceutical composition comprising: an LNP comprising an mRNA encoding a human OX40L polypeptide; and a pharmaceutically acceptable carrier; and (ii) by intravenous injection an effective amount of a PD-L1 antagonist; thereby treating ovarian cancer, or solid tumor, lymphoma or epithelial origin cancer, in the patient by inducing or enhancing an anti-tumor immune response.
E82. The method of embodiment 81, wherein the mRNA is administered at a dose of 1.0-8.0 mg in a dosing regimen from 7 to 21 days.
E83. The method of embodiment 82, wherein the mRNA is administered at a dose of 8.0 mg in a dosing regimen of once every two weeks or once every four weeks.
E84. The method of embodiment 81, wherein the PD-L1 antagonist is selected from the group consisting of durvalumab, avelumab, and atezolizumab.
E85. The method of embodiment 84, wherein the PD-L1 antagonist is durvalumab.
E86. The method of embodiment 85, wherein durvalumab is administered at a dose of 1500 mg in a dosing regimen of once every four weeks.
E87. The method of any one of embodiments 1-86, wherein the epithelial origin cancer is an epithelial cancer of ovary, fallopian tube or peritoneum.
E88. The method of any one of embodiments 1-87, wherein the patient has not responded to at least one prior anti-cancer treatment or at least one prior anti-cancer treatment has become ineffective.
E89. The method of embodiment 88, wherein the prior anti-cancer treatment is a chemotherapy treatment.
E90. The method of embodiment 88, wherein the prior anti-cancer treatment is a radiotherapy treatment.
E91. The method of embodiment 88, wherein the prior anti-cancer treatment is an immunotherapy treatment.
E92. A method of treating ovarian cancer in a human patient, the method comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating ovarian cancer in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg; and

(iv) the mRNA is administered in a dosing regimen selected from once every 7 to 28 days, once every 7 to 21 days, once every 7 to 14 days, once every 28 days, once every 21 days, once every 14 days and once every 7 days.

E93. A method of treating ovarian cancer in a human patient, the method comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating ovarian cancer in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E94. A method of treating a solid tumor in a human patient, the method comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating the solid tumor in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg; and

(iv) the mRNA is administered in a dosing regimen selected from once every 7 to 28 days, once every 7 to 21 days, once every 7 to 14 days, once every 28 days, once every 21 days, once every 14 days and once every 7 days.

E95. A method of treating a solid tumor in a human patient, the method comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating a solid tumor in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E96. A method of treating a lymphoma in a human patient, the method comprising administering to the patient an effective amount of a pharmaceutical composition comprising: a lipid nanoparticle (LNP) comprising a messenger RNA (mRNA) encoding a human OX40L polypeptide; and a pharmaceutically acceptable carrier, thereby treating the lymphoma in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg, optionally by intratumoral injection; and

(iv) the mRNA is administered in a dosing regimen selected from once every 7 to 28 days, once every 7 to 21 days, once every 7 to 14 days, once every 28 days, once every 21 days, once every 14 days and once every 7 days.

E97. A method of treating a lymphoma in a human patient, the method comprising administering to the patient an effective amount of a pharmaceutical composition comprising: a lipid nanoparticle (LNP) comprising a messenger RNA (mRNA) encoding a human OX40L polypeptide; and a pharmaceutically acceptable carrier, thereby treating the lymphoma in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg, optionally by intratumoral injection;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E98. A method of treating an epithelial origin cancer in a human patient, the method comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating the epithelial origin cancer in the patient by inducing or enhancing an anti-tumor immune response, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the mRNA is administered at a dose of 1.0 mg-8.0 mg; and

(iv) the mRNA is administered in a dosing regimen selected from once every 7 to 28 days, once every 7 to 21 days, once every 7 to 14 days, once every 28 days, once every 21 days, once every 14 days and once every 7 days.

E99. A method of treating an epithelial origin cancer in a human patient, the method comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating the epithelial origin cancer in the patient by inducing or enhancing an anti-tumor immune response, wherein:

(i) the mRNA comprises a nucleotide sequence at least 90% identical to the nucleotide sequence set forth in SEQ ID NO: 5;

(ii) the mRNA is administered at a dose of 1.0 mg-8.0 mg;

(iii) the mRNA is administered in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E100. The method of embodiment 98 or 99, wherein the epithelial origin cancer is an epithelial cancer of ovary, fallopian tube or peritoneum.
E101. The method of any one of embodiments 92-100, wherein the mRNA comprises an open reading frame at least 95% identical the nucleotide sequence set forth in SEQ ID NO: 4.
E102. The method of any one of embodiments 92-100, wherein the mRNA comprises an open reading frame comprising the nucleotide sequence set forth in SEQ ID NO: 4.
E103. The method of any one of embodiments 92-100, wherein the mRNA comprises a nucleotide sequence at least 95% identical the nucleotide sequence set forth in SEQ ID NO: 5.
E104. The method of any one of embodiments 92-100, wherein the mRNA comprises the nucleotide sequence set forth in SEQ ID NO: 5.
E105. The method of any one of embodiments 92-104, wherein the mRNA is administered at a dose of 8.0 mg.
E106. The method of any one of embodiments 92-105, wherein the mRNA is administered on day 1 and day 15 (±2 days) of a 28-day cycle for multiple cycles until the tumor lesion resolves or is administered on day 1 and day 15 (±2 days) of a 28-day cycle for one cycle and on day 1 of a 28-day cycle for multiple subsequent cycles until the tumor lesion resolves.
E107. The method of any one of embodiments 92-106, wherein the patient is also administered an immune checkpoint inhibitor.
E108. The method of embodiment 107, wherein the immune checkpoint inhibitor is an antagonist of PD-1/PD-L1 interaction.
E109. The method of embodiment 108, wherein the immune checkpoint inhibitor is a PD-1 antagonist.
E110. The method of embodiment 109, wherein the PD-1 antagonist is selected from the group consisting of nivolumab, pembrolizumab, and pidilizumab.
E111. The method of embodiment 108, wherein the immune check point inhibitor is a PD-L1 antagonist.
E112. The method of embodiment 111, wherein the PD-L1 antagonist is selected from the group consisting of durvalumab, avelumab, and atezolizumab.
E113. The method of embodiment 112, wherein the PD-L1 antagonist is durvalumab.
E114. The method of embodiment 113, wherein durvalumab is administered at a dose of 1500 mg in a dosing regimen of once every four weeks.
E115. A kit comprising a container comprising a pharmaceutical composition comprising: a lipid nanoparticle comprising an mRNA encoding a human OX40L polypeptide; and a pharmaceutically acceptable carrier, wherein the pharmaceutical composition comprises 2 mg/ml of the mRNA, and a package insert comprising instructions for administration of the mRNA by intratumoral injection to treat or delay progression of ovarian cancer, or a solid tumor, lymphoma or epithelial origin cancer, in a human patient
E116. A kit comprising a container comprising a pharmaceutical composition comprising: a lipid nanoparticle comprising an mRNA encoding a human OX40L polypeptide; and a pharmaceutically acceptable carrier, wherein the pharmaceutical composition comprises 2 mg/ml of the mRNA, and a package insert comprising instructions for administration of the mRNA by intratumoral injection and instruction for use in combination with a second composition comprising a PD-1 antagonist, a PD-L1 antagonist or a CTLA-4 antagonist, for use in treating or delaying progression of ovarian cancer, or a solid tumor, lymphoma or epithelial origin cancer, in a human patient.
E117. The kit of any one of embodiments 115-116, wherein the instructions provide administration of the lipid nanoparticle in a dosing regimen selected from 7 to 28 days, 7 to 21 days, 7 to 14 days, 28 days, 21 days, 14 days and 7 days.
E118. The kit of any one of embodiments 115-116, wherein the instructions provide administration of the lipid nanoparticle every 2 weeks in a 28-day cycle.
E119. The kit of any one of embodiments 115-118, wherein the human OX40L polypeptide comprises the amino acid sequence set forth in SEQ ID NO: 1.
E120. The kit of any one of embodiments 115-119, wherein the mRNA comprises an open reading frame comprising a nucleotide sequence at least 90% identical to the nucleotide sequence set forth in SEQ ID NO: 4.
E121. The kit of any one of embodiments 115-119, wherein the mRNA comprises an open reading frame comprising the nucleotide sequence set forth in SEQ ID NO: 4.
E122. The kit of any one of embodiments 115-121, wherein the mRNA comprises a 3′ untranslated region (UTR) comprising at least one microRNA-122 (miR-122) binding site.
E123. The kit of embodiment 122, wherein the miR-122 binding site is a miR-122-3p binding site.
E124. The kit of embodiment 122, wherein the miR-122 binding site is a miR-122-5p binding site.
E125. The kit of embodiment 124, wherein the miR-122-5p binding site comprises the nucleotide sequence set forth in SEQ ID NO: 20.
E126. The kit of embodiment 124, wherein the 3′UTR comprises the nucleotide sequence set forth in SEQ ID NO: 17.
E127. The kit of embodiment 122, wherein the mRNA comprises a 5′ untranslated region (UTR) comprising the nucleotide sequence set forth in SEQ ID NO: 15.
E128. The kit of embodiment 122, wherein the mRNA comprises a 5′ cap.
E129. The kit of embodiment 122, wherein the mRNA comprises a poly-A tail of about 100 nucleotides in length.
E130. The kit of any one of embodiments 115-129, wherein the mRNA comprises a nucleotide sequence at least 90% identical to the nucleotide sequence set forth in SEQ ID NO: 5.
E131. The kit of any one of embodiments 115-129, wherein the mRNA comprises the nucleotide sequence set forth in SEQ ID NO: 5.
E132. The kit of any one of embodiments 115-131, wherein the mRNA is chemically modified.
E133. The kit of embodiment 132, wherein the mRNA is fully modified with chemically-modified uridines.
E134. The kit of embodiment 133, wherein the chemically-modified uridines are N1-methylpseudouridines (m1ψ).
E135. The kit of embodiment 133, wherein the mRNA is fully modified with 5-methylcytosine or is fully modified with N1-methylpseudouridines (m1ψ) and 5-methylcytosine.
E136. The kit of any one of embodiments 115-135, wherein the LNP comprises a compound having the formula:

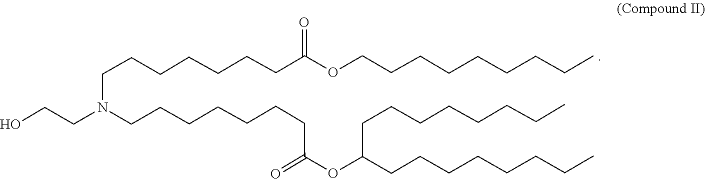 [SMILES: CCCCCCCCCOC(=O)CCCCCCCN(CCO)CCCCCCCC(=O)OC(CCCCCCCC)CCCCCCCC]

E137. The kit of embodiment 136, wherein the LNP further comprising a phospholipid, a structural lipid, and a PEG lipid.
E138. The kit of any one of embodiments 115-135, wherein the LNP comprises a molar ratio of about 20-60% ionizable amino lipid, about 5-25% phospholipid, about 25-55% structural lipid, and about 0.5-1.5% PEG lipid.
E139. The kit of embodiment 138, wherein the LNP comprises a molar ratio of about 50% ionizable amino lipid, about 10% phospholipid, about 38.5% structural lipid, and about 1.5% PEG lipid.
E140. The kit of embodiment 138, wherein the LNP comprises a molar ratio of about 50% ionizable amino lipid, about 10% phospholipid, about 38.5% cholesterol, and about 1.5% PEG-DMG.
E141. The kit of any one of embodiments 138-140, wherein the ionizable amino lipid comprises a compound having the formula:

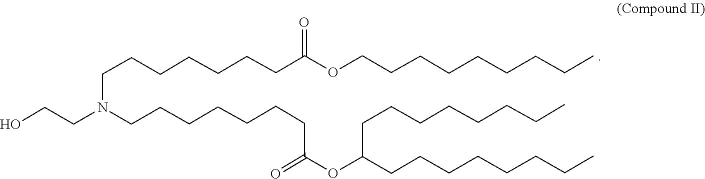 [SMILES: CCCCCCCCCOC(=O)CCCCCCCN(CCO)CCCCCCCC(=O)OC(CCCCCCCC)CCCCCCCC]

E142. The kit of embodiment 116, wherein the PD-1 antagonist is an antibody or antigen binding portion thereof that specifically binds to PD-1.
E143. The kit of embodiment 116, wherein the PD-L1 antagonist is an antibody or antigen binding portion thereof that specifically binds to PD-L1.
E144. The kit of embodiment 116, wherein the CTLA-4 antagonist is an antibody or antigen binding portion thereof that specifically binds to CTLA-4.
E145. The kit of embodiment 142, wherein the PD-1 antagonist is selected from the group consisting of nivolumab, pembrolizumab, and pidilizumab.
E146. The kit of embodiment 143, wherein the PD-L1 antagonist is selected from the group consisting of durvalumab, avelumab, and atezolizumab.
E147. The kit of embodiment 146, wherein the PD-L1 antagonist is durvalumab.
E148. The kit of embodiment 147, wherein instructions provide for administration of durvalumab at a dose of 1500 mg.
E149. The kit of embodiment 144, wherein the CTLA-4 antagonist is selected from the group consisting of ipilimumab and tremelimumab.
E150. The kit of any one of embodiments 115-149, wherein instructions provide for administration of mRNA at a dose of 1.0-8.0 mg.
E151. The kit of embodiment 150, wherein instructions provide for administration of mRNA at a dose of 8.0 mg.
E152. The kit of any one of embodiments 115-151, wherein the epithelial origin cancer is an epithelial cancer of ovary, fallopian tube or peritoneum.
E153. A kit for the treatment of ovarian cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of ovarian cancer in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

E154. A kit for the treatment of ovarian cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of ovarian cancer in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E155. A kit for the treatment of a solid tumor in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the solid tumor in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

E156. A kit for the treatment of a solid tumor in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the solid tumor in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E157. A kit for the treatment of a lymphoma in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA to treat or delay progression of the lymphoma in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg, optionally by intratumoral injection; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

E158. A kit for the treatment of a lymphoma in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA to treat or delay progression of the lymphoma in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg, optionally by intratumoral injection;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E159. A kit for the treatment of an epithelial origin cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the epithelial origin cancer in a human patient, wherein:

(i) the mRNA comprises an open reading frame comprising a nucleotide sequence at least 90% identical to the nucleotide sequence set forth in SEQ ID NO: 4;

(ii) the LNP comprises an ionizable amino lipid, a phospholipid, a structural lipid and a PEG lipid, wherein the ionizable amino lipid is Compound II and optionally wherein the LNP comprises a molar ratio of about 20-60% ionizable amino lipid, about 5-25% phospholipid, about 25-55% structural lipid, and about 0.5-1.5% PEG lipid;

(iii) the package insert instructs administration of the mRNA at a dose of 1.0 mg-8.0 mg; and

(iv) the package insert instructs administration of the mRNA in a dosing regimen selected from once every 7 to 28 days, once every 7 to 21 days, once every 7 to 14 days, once every 28 days, once every 21 days, once every 14 days and once every 7 days.

E160. A kit for the treatment of an epithelial origin cancer in a human patient, the kit comprising a pharmaceutical composition comprising: a lipid nanoparticle (LNP) comprising a messenger RNA (mRNA) encoding a human OX40L polypeptide; and a pharmaceutically acceptable carrier, and a package insert comprising instructions for administration of the mRNA by intratumoral injection to treat or delay progression of the epithelial origin cancer in a human patient, wherein:

(i) the mRNA comprises a nucleotide sequence at least 90% identical to the nucleotide sequence set forth in SEQ ID NO: 5;

(ii) the package insert instructs administration of the mRNA at a dose of 1.0 mg-8.0 mg;

(iii) the package insert instructs administration of the mRNA in a dosing regimen selected from once every 7 to 28 days, once every 7 to 21 days, once every 7 to 14 days, once every 28 days, once every 21 days, once every 14 days and once every 7 days; and

(iv) optionally, the LNP comprises an ionizable amino lipid, a phospholipid, a structural lipid and a PEG lipid, wherein optionally the ionizable amino lipid is Compound II and wherein optionally the LNP comprises a molar ratio of about 20-60% ionizable amino lipid, about 5-25% phospholipid, about 25-55% structural lipid, and about 0.5-1.5% PEG lipid.

E161. The kit of embodiment 159 or 160, wherein the epithelial origin cancer is an epithelial cancer of ovary, fallopian tube or peritoneum.
E162. The kit of any one of embodiments 153-160, wherein the mRNA comprises an open reading frame at least 95% identical the nucleotide sequence set forth in SEQ ID NO: 4.
E163. The kit of any one of embodiments 153-160, wherein the mRNA comprises an open reading frame comprising the nucleotide sequence set forth in SEQ ID NO: 4.
E164. The kit of any one of embodiments 153-160, wherein the mRNA comprises a nucleotide sequence at least 95% identical the nucleotide sequence set forth in SEQ ID NO: 5.
E165. The kit of any one of embodiments 153-160, wherein the mRNA comprises the nucleotide sequence set forth in SEQ ID NO: 5.
E166. The kit of any one of embodiments 153-165, wherein the package insert instructs administration of the mRNA at a dose of 8.0 mg.
E167. The kit of any one of embodiments 153-166, wherein the package insert instructs administration of the mRNA on day 1 and day 15 (±2 days) of a 28-day cycle for multiple cycles until the tumor lesion resolves or on day 1 and day 15 (±2 days) of a 28-day cycle for one cycle and on day 1 of a 28-day cycle for multiple subsequent cycles until the tumor lesion resolves.
E168. The kit of any one of embodiments 153-167, wherein the package insert instructs administration of an immune checkpoint inhibitor in combination with the mRNA.
E169. The kit of embodiment 168, wherein the immune checkpoint inhibitor is an antagonist of PD-1/PD-L1 interaction.
E170. The kit of embodiment 169, wherein the immune checkpoint inhibitor is a PD-1 antagonist.
E171. The kit of embodiment 170, wherein the PD-1 antagonist is selected from the group consisting of nivolumab, pembrolizumab, and pidilizumab.
E172. The kit of embodiment 169, wherein the immune check point inhibitor is a PD-L1 antagonist.
E173. The kit of embodiment 172, wherein the PD-L1 antagonist is selected from the group consisting of durvalumab, avelumab, and atezolizumab.
E174. The kit of embodiment 173, wherein the PD-L1 antagonist is durvalumab.
E175. The kit of embodiment 174, wherein the package insert instructs administration of durvalumab at a dose of 1500 mg in a dosing regimen of once every four weeks.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1: Clinical Study Design to Evaluate Anti-Tumor Efficacy of mRNA Encoding Human OX40L in Human Cancer Patients

Inhibition or blockade of co-inhibitory immune checkpoints has become a standard of treatment for diverse solid and hematologic malignancies. However, pharmacological checkpoint inhibition is often not sufficient to induce robust and durable tumor regressions in patients. Generation of optimal anti-tumor T cell responses requires T cell receptor activation and T cell co-stimulation, the latter of which is induced via ligation of tumor necrosis factor (TNF) receptor family members, such as OX40.

As described further herein, the OX40 receptor (alternatively known as TNFRSF4, cluster of differentiation [CD]134) is expressed on activated immune effector cells such as T cells and natural killer (NK) cells (Compaan and Hymowitz (2006) Structure 14(8):1321-1330). The ligand of OX40 (OX40L) is a homo-trimeric transmembrane protein normally expressed on antigen-presenting cells upon immune stimulation (Mallett et al., (1990) EMBO J 9(4):1063-1068). Binding of OX40 and OX40L in the presence of a recognized antigen (e.g., a tumor antigen) promotes the expansion of CD4+ and CD8+ T cells and enhances memory responses while inhibiting regulatory T cells. Expression of OX40L by tumor cells, or other cells presenting tumor antigens is known to induce cell-mediated immune responses with systemic anti-tumor effects.

In preclinical tumor models, durable tumor regression was observed following intratumoral (i.tu.) administration of an mRNA encoding OX40L (data not shown). To evaluate the anti-tumor efficacy of an mRNA encoding human OX40L in human cancer patients, a lipid nanoparticle-encapsulated mRNA encoding human OX40L was administered intratumorally to patients with advanced relapsed/refractory solid tumor malignancies or lymphoma, including two patients with ovarian carcinoma, in a clinical (phase 1) dose-escalation clinical study. Briefly, the mRNA encoding human OX40L was formulated in a lipid nanoparticle comprising Compound II, and the lipid nanoparticles were formulated in a buffer solution suitable for injection.

Specifically, mRNA encoding human OX40L comprised an open reading frame having the nucleotide sequence set forth in SEQ ID NO: 4, a 5′UTR having the nucleotide sequence set forth in SEQ ID NO: 16, and a 3′UTR comprising a miR-122 binding site having the nucleotide sequence set forth in SEQ ID NO: 17. The mRNA was formulated in an lipid nanoparticle comprising a molar ratio of 50% Compound II, 10% DSPC, 38.5% cholesterol, and 1.5% PEG-DMG.

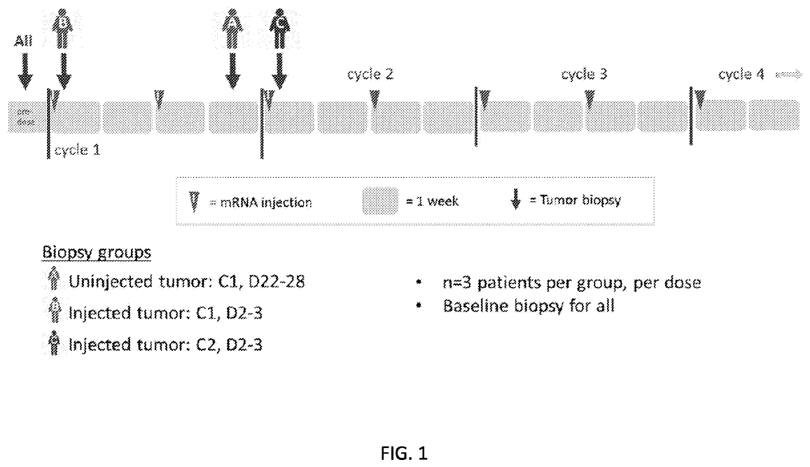

The lipid nanoparticle was administered to 26 patients via intratumoral injection on days 1 and 15 (±2 days) of multiple 28-day cycles (FIG. 1). On cycle 1, day 1 (C₁D1) an initial dose of 1.0 mg of the formulated OX40L mRNA was administered to patients. A subsequent dose was administered on day 15, then again on day 1 and day 15 of subsequent cycles, as indicated (‘mRNA injection’) in FIG. 1. Depending on the patient, subsequent dosages of 2.0, 4.0, and 8.0 mg dose levels were administered on subsequent scheduled days. A subset of patients treated with intratumoral injection of formulated OX40L mRNA, as well as control patients with untreated tumors, were enrolled into one of three biopsy cohorts, as indicated in FIG. 1:

Cohort A: Patients underwent a first (baseline) biopsy of the untreated tumor followed by second biopsy of the untreated tumor between day 22 and day 28 within cycle 1 (C1D22-28).

Cohort B: Patients underwent a first (baseline) biopsy of the primary tumor to be treated followed by a second biopsy of the treated tumor occurring 24 to 48 hours post-injection after day 1 within cycle 1 (C1D1).

Cohort C: Patients underwent a first (baseline) biopsy of the primary tumor to be treated followed by a second biopsy of the treated tumor occurring 24 to 48 hours post-injection after day 1 within cycle 2 (C2D1).

As described further in the Examples below, biopsy material from each cohort was used to determine an expression level of biomarkers via multiplexed Quantitative Immunofluorescence (mQIF) analysis and to determine an extent of tumor T cell infiltration. Clinical observations were generated using computerized tomography (CT) scans as well as visual and palpable evaluations of cutaneous/subcutaneous tumor lesions. Further patient data, including demographics, disease and treatment history, and any adverse events following administration of OX40L mRNA was collected during the course of the study (data not shown).

### Example 2: Anti-Tumor Efficacy of mRNA Encoding Human OX40L in Humans with Ovarian Carcinoma

The anti-tumor efficacy of mRNA encoding human OX40L was evaluated in two patients (referred to as 009-001 and 007-002) with ovarian carcinoma.

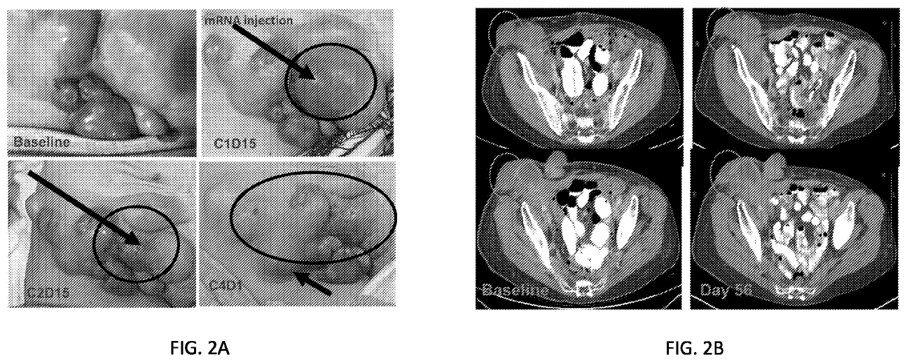

Patient 009-001, a 63 year old female diagnosed in 2003 with Stage 3 serous ovarian carcinoma previously treated with multiple surgeries of curative intent and debulking intermixed with 13 rounds of chemotherapy and hormone therapy, was administered an initial dose (C₁D1) of formulated OX40L mRNA at 2 mg/dose followed by 6 subsequent doses formulated OX40L at 2 mg/dose. At baseline, patient 009-001 presented with a complex subcutaneous tumor nest comprising multiple tumors (FIG. 2A, top left panel). At the time of the 4th dose (C₂D15), patient 009-001 presented with an obvious reduction in the size of the injected tumor lesion as well as visual and palpable improvement in local regional non-injected tumor lesions (FIG. 2A, bottom left panel). Further improvement in injected and non-injected tumor lesions at the time of the 7th dose (C4D1) is shown in FIG. 2A, bottom right panel). An adjacent uninjected lesion was biopsied at C1D27 showed signs of clinical improvement, as indicated by softening and flatting against the abdominal wall upon physical exam. Transverse abdominal/pelvis CT scan images of the tumor lesions presented by patient 009-001 (FIG. 2B), shows a reduction in tumor size at day 56 post-initial dose (FIG. 2B, right panels) relative to baseline (FIG. 2B, left panels). Although the responses of patient 009-001 to treatment do not meet RECIST criteria for PR (partial response), primarily due to the complex nature of the tumor nest, the initial injection lesion visually resolved requiring movement of the injection site to an adjacent lesion (FIG. 2A, bottom right panel).

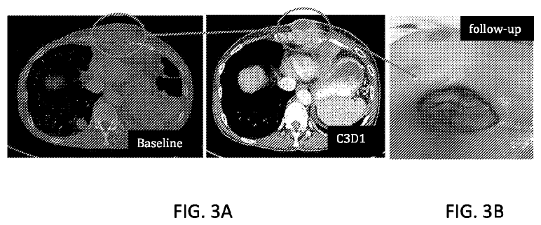

Patient 007-002, a 60 year old female diagnosed in 2012 with high-grade serous ovarian carcinoma previously treated with multiple surgeries of curative intent with 7 rounds of chemotherapy and palliative chest wall radiation, was administered an initial dose (C1D1) of formulated OX40L mRNA at 1 mg/dose followed by 4 subsequent doses formulated OX40L at 1 mg/dose. Patient 007-002 had a large sternal lesion which had eroded through the sternum measuring 6.3 cm at baseline (FIG. 3A, right panel). At the time of first restaging scan (˜56 days post-initial dose) (FIG. 3A, left panel), the sternal lesion had decreased in length to 4.6 cm, after that the patient withdrew from study for personal reasons. During the time after the patient withdrew from study, it was recorded that the injected tumor had begun to decrease in size as she was followed by wound clinic for a small ulcer <0.5 cm at the injection site which had virtually resolved before going on salvage chemotherapy with cyclophosphamide & bevacizumab. The ulcer, which had been noted to be with the wound base not visible prior to salvage chemotherapy, rapidly enlarged after she was treated with salvage chemotherapy and showed complete resolution of the injected tumor (FIG. 3B).

These results show that intratumoral injection of ovarian cancer tumor with an mRNA encoding human OX40L provides direct and systemic anti-tumor effects, as indicated by a reduction in the size of injected tumors, indicating a direct anti-tumor response, and a reduction in the size of distal uninjected tumors, indicating a local regional abscopal effect.

### Example 3: Expression of OX40L in Ovarian Tumors Following Intratumoral Administration of mRNA Encoding Human OX40L

To determine if intratumoral administration of mRNA encoding human OX40L increases expression of OX40L in the tumor or tumor microenvironment, biopsies of the ovarian tumor from patient 007-002 were evaluated for OX40L expression prior to and then after injection by quantitative immunofluorescence analysis (QIF). Briefly, pre- and post-treatment biopsy samples were formalin-fixed and paraffin embedded (FFPE), and five-micron thick sections were immunofluorescently stained with multiplex panels of antibodies and analyzed by QIF (via AQUA scoring, essentially as described in McCabe et al., (2005) J Natl Cancer Inst 97(24):1808-1815) to evaluate the spatial patterns of OX40L protein expressed by the OX40L mRNA and to quantify OX40L expression levels.

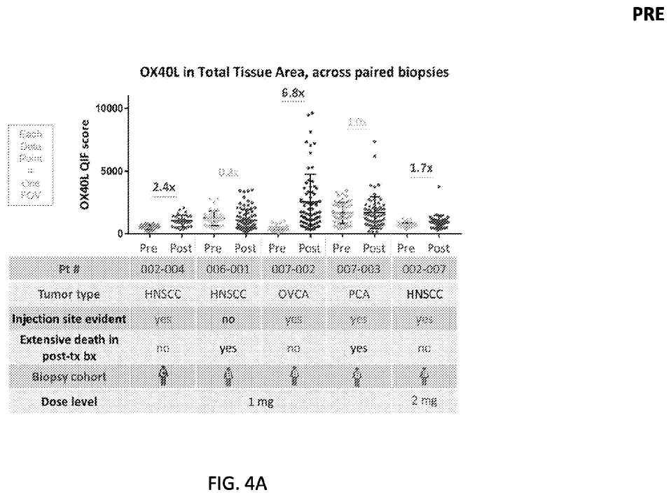

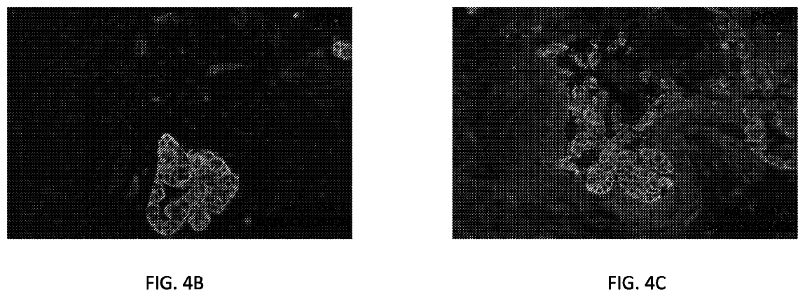

A summary of OX40L scores across five paired biopsies collected from patients in biopsy cohorts B and C is shown in FIG. 4A. Increases in OX40L expression following administration of OX40L mRNA was observed for patients 002-004, 007-002, and 002-007. FIG. 4B shows a representative immunofluorescence image showing localized, focal increases in OX40L expression at one day post-C1D1 dose in injected tumor relative to baseline in patient 007-002. (AQ=OX40L AQUA score; DAPI=DNA nuclear stain 4′,6-diamidino-2-phenylindole; CK=cytokeratin)

These results demonstrate that patient 007-002 has elevated OX40L expression in the post-treatment biopsy collected from two days after injection of 1 mg of formulated OX40L mRNA as compared to baseline as determined by QIF.

Collectively, the results shown in Examples 2 and 3 show that intratumoral administration of an mRNA encoding human OX40L into ovarian tumors results in direct and local regional abscopal anti-tumor efficacy, as indicated by a reduction in size in the injected tumor and non-injected tumors. Further these results demonstrate that intratumoral administration of an mRNA encoding human OX40L results in increased expression of human OX40L polypeptide in the tumor and tumor microenvironment, as indicated by QIF analysis of tumor biopsies taken pre- and post-treatment.

### Example 4: Clinical Study Design to Evaluate Anti-Tumor Efficacy of mRNA Encoding Human OX40L in Human Cancer Patients Alone or in Combination with an Immune Checkpoint Inhibitor

The combination of OX40L mRNA and an immune checkpoint inhibitor has been demonstrated to be effective in inhibiting tumor growth in an animal model. In particular, intratumoral injection of an LNP-encapsulated OX40L mRNA in combination with intravenous anti-PD-1 antibody treatment has been shown to inhibit tumor growth in an MC38 colon adenocarcinoma model in mice as reported in PCT Publication WO 2017/112943 (see Examples 18-19 and FIGS. 20-22).

A clinical study was designed to compare the effect of intratumoral injection of mRNA encoding human OX40L (hOX40L) alone or with treatment in combination with an immune checkpoint inhibitor in human patients. The immune checkpoint inhibitor used is the anti-PD-L1 antibody durvalumab. Patients to be treated include those with solid tumors, lymphomas or ovarian cancer, including cancers of epithelial origin of the ovary, the fallopian tube or the peritoneum. Solid malignancies include but are not limited to melanoma, breast cancer, head and neck cancer squamous cell carcinoma. Lymphomas include but are not limited to diffuse large B cell lymphoma. Malignancies include but are not limited to locally advanced, recurrent or metastatic tumors.

For mRNA treatment, LNP encapsulated hOX40L mRNA is injected directly into the tumor. For visible or palpable tumors, the tumors are easily injected without the use of imaging guidance. For visceral lesions, intratumor injection is achieved using ultrasound or computer tomography (CT) guidance. Preferably, the mRNA is administered in a single injection; however, multiple injections into different sites within the same lesion or split across several lesions are used when no single lesion is available that is large enough to receive the entire dose in the maximum injection volume per lesion size. LNP-encapsulated hOX40L typically is formulated at 2.0 mg/ml. Thus, injection doses of 1 mg, 2 mg, 4 mg or 8 mg correspond to injection volumes of 0.5 ml, 1.0 ml, 2.0 ml or 4.0 ml, respectively.

For patients treated with mRNA alone, mRNA encoding human OX40L is formulated in a lipid nanoparticle comprising Compound II, and the lipid nanoparticles are formulated in a buffer solution suitable for injection. For all patients, the lipid nanoparticles are administered via intratumoral injection on days 1 and 15 (±2 days) of cycle 1 of multiple 28-day cycles. For patients with superficial lesions, mRNA also is injected on days 1 and 15 for subsequent cycles (cycles 2-6). For patients with visceral lesions, mRNA is only injected on day 1 of subsequent cycles (2-6). A dose escalation study is performed using doses of 1.0, 2.0, 4.0, or 8.0 mg of the formulated OX40L mRNA.

For the combination treatment cohort, mRNA treatment is as described above for mRNA treatment alone except that for all patients, mRNA treatment is on days 1 and 15 for cycle 1 and only on day 1 for subsequent cycles (cycles 2-6). Anti-PD-L1 combination therapy comprises administration of durvalumab (Astrazeneca/MedImmune) intravenously at a dose of 1500 mg on day 1 of each 28 day cycle. mRNA treatment is at an initial dose of 4.0 mg, although lower (2.0 mg) or higher (8.0 mg) doses are chosen for subsequent cycles based on patient evaluation.

Patient evaluation includes tumor assessments (e.g., CT, MRI or PET-CT imaging) and pharmacokinetic/pharmacodynamic assessments including cytokine profile sampling and serum immunogenicity sampling.

### Example 5: Gene Expression in Ovarian Tumors Following Intratumoral Administration of mRNA Encoding Human OX40L

mRNA encoding human OX40L is being tested in an open-label, multicenter, Phase 1/2 study of repeated intratumoral injections in patients with advanced relapsed/refractory solid tumor malignancies or lymphoma. The study includes 3 dosing periods: a dose escalation in non-visceral lesions followed by a dose confirmation in visceral lesions and an expansion at the MTD (maximum tolerated dose)/RDE (recommended dose for expansion) in ovarian cancer of epithelial origin.

The mRNA encoding human OX40L was formulated in a lipid nanoparticle comprising Compound II, and the lipid nanoparticles were formulated in a buffer solution suitable for injection. The LNP-encapsulated mRNA was administered to patients as intratumoral injections in accessible lesions on Days 1 and 15 of each 28-day cycle (±2 days). The starting dose is 1.0 mg mRNA, and 2.0, 4.0, and 8.0 mg dose levels (corresponding to cohorts 1, 2, 3, and 4, respectively) are also being evaluated during this trial.

Two independent, non-overlapping immune signature scores, gene expression profiling (GEP) score and cytolytic activity (CYT) score, were utilized to assess baseline tumor microenvironment (TME) status, and the effect of human OX40L treatment on the TME. The T cell-inflamed GEP score, largely consisting of IFN-γ-responsive genes (Ayers et al., 2017; Cristescu et al., 2018). IFN-γ is the only member of the type II interferon (IFN-II) class and is produced by activated lymphocytes. The GEP score was developed using expression data from 18 specific genes as measured using the NanoString platform; expression measurements of these genes in the analyses are from RNA-seq data. The CYT score measures immune cytolytic activity based on transcript levels of two key cytolytic effectors, granzyme A (GZMA) and perforin (PRF1), which are upregulated upon CD8⁺ cytotoxic T, NK, and/or NKT cell activation, and associate with productive anti-tumor responses (Rooney et al., 2015; Herbst et al, 2014). An additional panel of genes was evaluated for a broader view of T cell and dendritic cell (DC) abundance and activation (Danaher et al, 2017; Hewitt et al, 2019), as well as interferon type I (IFN-I) responses (Yao et al, 2009). IFN-I's are activated by DAMPs and PAMPs (Damage Associated- and Pathogen Associated Molecular Patterns, respectively), and signal through distinct receptors from interferon type II (IFN-II). Enrichment for several different IFN-I gene sets relative to a comprehensive compendium of gene sets (GO: gene ontology resource comprised of 5,917 different gene sets related to a variety of biological systems; Ashburner et al 2000; The Gene Ontology Consortium 2019) was also assessed by single-sample Gene Set Enrichment Analysis (ssGSEA, Table 5). Changes in IFN-I signaling is of interest due to the potential for immunostimulatory components present in the OX40L mRNA to activate these pathways.

TABLE 5
Ranking of enrichment score changes of IFN-I gene sets in the Gene Ontology (GO) gene set collection.

| | | Patient | Patient | Patient | Patient | Patient | Patient | Patient | Patient | Patient |
|---|---|---|---|---|---|---|---|---|---|---|
| Gene set | Gene # | 006-001 | 007-002 | 007-003 | 002-005 | 002-004 | 008-006 | 002-007 | 009-001 | 009-004 |
| Pos Reg of IFN-I | 70 | 50.7% | 85.4% | 79.3% | 52.7% | 56.5% | 92.8% | 83.7% | 60.0% | 36.0% |
| Neg Reg of IFN-I | 40 | 52.8% | 87.8% | 90.9% | 59.1% | 47.5% | 98.3% | 90.8% | 16.7%* | 47.0% |
| Resp to IFN-I | 68 | 82.6% | 84.6% | 98.7% | 55.5% | 53.7% | 99.4% | 99.3% | 4.0%* | 9.4%* |
| Reg of IFN-I Signaling | 39 | 57.4% | 79.9% | 69.2% | 44.9% | 31.9% | 89.6% | 87.7% | 26.2%* | 55.2% |
| IFN-I Receptor Binding | 17 | 17.7%* | 14.1%* | 65.3% | 2.6%* | 40.5% | 96.6% | 41.5% | 8.3%* | 66.3% |
| Midpoint | | 50.8% | 42.8% | 31.9% | 36.5% | 27.2% | 36.0% | 35.6% | 58.4% | 41.1% |

Enrichment scores were calculated between all samples and all gene sets (n = 5,917) in the GO collection using ssGSEA. The score changes from pre- to post-treatment were calculated and ranked from smallest to largest of all gene sets for each patient with paired biopsies. The ranked percentiles from low to high indicate reduced to elevated enrichment score change from pre- to post- treatment. For example, a score of 99.4% for the ‘Response to IFN-I’ gene set indicates that the enrichment score increase for this gene set post- treatment was greater than 99.4% of the 5917 GO gene sets tested. The percentile of gene set enrichment score changes was calculated case by case. Midpoint = the percentile of midpoint gene sets for each patient; for each case, the percentile of the gene set with the enrichment score change closest to 0 was listed as midpoint. The cells with an asterisk and the cells underlined indicate the gene set enrichment score changes from decrease to increase, with the unmarked cells representing the ranking of gene sets with almost no change post treatment.

Cases with most pronounced levels of human OX40L in post-treatment biopsies collected from injected lesions, demonstrated elevated GEP and CYT scores, as well as broad increases of other transcripts indicative of T cell and DC cell activation. IFN-I activation was evident in most cases at both the single gene and at the gene set level by analysis of a 21-gene IFNα/β-induced gene set, and from evaluation of IFN-I associated gene sets in the GO resource (Table 5; IFN-I gene set enrichment rankings relative to other gene sets in GO, based on the differential of IFN-I enrichment scores in pre- versus post-treatment samples).

Major observations from this interim dataset include activation of a pro-inflammatory response post-treatment in multiple cases, including demonstration of IFN-I responses. Increased T-cell inflamed GEP scores and CYT scores were observed post-treatment in 5/9 and 6/9 paired biopsies evaluated, respectively. Many of the cases with evidence of a pro-inflammatory response by RNAseq also exhibited increased T cells by QIF. PD-L1 was also elevated in post-treatment biopsies at the transcript and/or modestly at the protein level in several cases. In summary, changes to the tumor microenvironment following treatment suggest an inflammatory effect of OX40L mRNA.

### Sequence Summary

| SEQ | |
|---|---|
| ID | |
| NO: | DESCRIPTION |
| 1 | MERVQPLEENVGNAARPRFERNKLLLVASVIQGLGLLLCFTYICLHFSALQVSHRYPRIQSIKVQFTE |
| | YKKEKGFILTSQKEDEIMKVQNNSVIINCDGFYLISLKGYFSQEVNISLHYQKDEEPLFQLKKVRSVN |
| | SLMVASLTYKDKVYLNVTTDNTSLDDFHVNGGELILIHQNPGEFCVL |
| | OX40L (TNSFR4)-Tumor necrosis factor ligand superfamily member 4 |
| | isoform 1 [Homo sapiens] NP_003317 (bold is intracellular domain, |
| | italics is transmembrane domain, and underline is extracellular |
| | domain) |
| 2 | MVSHRYPRIQSIKVQFTEYKKEKGFILTSQKEDEIMKVQNNSVIINCDGFYLISLKGYFSQEVNISLH |
| | YQKDEEPLFQLKKVRSVNSLMVASLTYKDKVYLNVTTDNTSLDDFHVNGGELILIHQNPGEFCVL |
| | OX40L (TNSFR4)-TNFSF4 isoform 2 [Homo sapiens] NP_001284491 |
| 3 | MEGEGVQPLDENLENGSRPRFKWKKTLRLVVSGIKGAGMLLCFIYVCLQLSSSPAKDPPIQRLRGAVT |
| | RCEDGQLFISSYKNEYQTMEVQNNSVVIKCDGLYIIYLKGSFFQEVKIDLHFREDHNPISIPMLNDGR |
| | RIVFTVVASLAFKDKVYLTVNAPDTLCEHLQINDGELIVVQLTPGYCAPEGSYHSTVNQVPL |
| | OX40L (TNSFR4)-TNFSF4 [Mus musculus] NP_033478 |
| 4 | AUGGAAAGGGUCCAACCCCUGGAAGAGAAUGUGGGAAAUGCAGCCAGGCCAAGAUUCGAGAGGAACAA |
| | GCUAUUGCUGGUGGCCUCUGUAAUUCAGGGACUGGGGCUGCUCCUGUGCUUCACCUACAUCUGCCUGC |
| | ACUUCUCUGCUCUUCAGGUAUCACAUCGGUAUCCUCGAAUUCAAAGUAUCAAAGUACAAUUUACCGAA |
| | UAUAAGAAGGAGAAAGGUUUCAUCCUCACUUCCCAAAAGGAGGAUGAAAUCAUGAAGGUGCAGAACAA |
| | CUCAGUCAUCAUCAACUGUGAUGGGUUUUAUCUCAUCUCCCUGAAGGGCUACUUCUCCCAGGAAGUCA |
| | ACAUUAGCCUUCAUUACCAGAAGGAUGAGGAGCCCCUCUUCCAACUGAAGAAGGUCAGGUCUGUCAAC |
| | UCCUUGAUGGUGGCCUCUCUGACUUACAAAGACAAAGUCUACUUGAAUGUGACCACUGACAAUACCUC |
| | CCUGGAUGACUUCCAUGUGAAUGGCGGAGAACUGAUUCUUAUCCAUCAAAAUCCUGGUGAAUUCUGUG |
| | UCCUU |
| | Human OX40L mRNA (ORF) |
| 5 | 5′^{7Me}GₚₚₚG_{2′Ome}GGAAAUAAGAGAGAAAAGAAGAGUAAGAAGAAAUAUAAGAGCCACCAUGGAAAGGGU |
| | CCAACCCCUGGAAGAGAAUGUGGGAAAUGCAGCCAGGCCAAGAUUCGAGAGGAACAAGCUAUUGCUGG |
| | UGGCCUCUGUAAUUCAGGGACUGGGGCUGCUCCUGUGCUUCACCUACAUCUGCCUGCACUUCUCUGCU |
| | CUUCAGGUAUCACAUCGGUAUCCUCGAAUUCAAAGUAUCAAAGUACAAUUUACCGAAUAUAAGAAGGA |
| | GAAAGGUUUCAUCCUCACUUCCCAAAAGGAGGAUGAAAUCAUGAAGGUGCAGAACAACUCAGUCAUCA |
| | UCAACUGUGAUGGGUUUUAUCUCAUCUCCCUGAAGGGCUACUUCUCCCAGGAAGUCAACAUUAGCCUU |
| | CAUUACCAGAAGGAUGAGGAGCCCCUCUUCCAACUGAAGAAGGUCAGGUCUGUCAACUCCUUGAUGGU |
| | GGCCUCUCUGACUUACAAAGACAAAGUCUACUUGAAUGUGACCACUGACAAUACCUCCCUGGAUGACU |
| | UCCAUGUGAAUGGCGGAGAACUGAUUCUUAUCCAUCAAAAUCCUGGUGAAUUCUGUGUCCUUUGAUAA |
| | UAGGCUGGAGCCUCGGUGGCCAUGCUUCUUGCCCCUUGGGCCUCCCCCCAGCCCCUCCUCCCCUUCCU |
| | GCACCCGUACCCCCCAAACACCAUUGUCACACUCCAGUGGUCUUUGAAUAAAGUCUGAGUGGGCGGCA |
| | AAAAAAAAAAAAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAUCUAG_{OH}3′ |
| | Where: A,C G & U = AMP, CMP, GMP & N1-ΨUMP, respectively; |
| | Me = methyl; p = inorganic phosphate |
| | Full-length mRNA Nucleotide sequence (5′ UTR, ORF, 3′ UTR, polyA |
| | tail) of human OX40L |
| 6 | GGCCCUGGGACCUUUGCCUAUUUUCUGAUUGAUAGGCUUUGUUUUGUCUUUACCUCCUUCUUUCUGGG |
| | GAAAACUUCAGUUUUAUCGCACGUUCCCCUUUUCCAUAUCUUCAUCUUCCCUCUACCCAGAUUGUGAA |
| | GAUGGAAAGGGUCCAACCCCUGGAAGAGAAUGUGGGAAAUGCAGCCAGGCCAAGAUUCGAGAGGAACA |
| | AGCUAUUGCUGGUGGCCUCUGUAAUUCAGGGACUGGGGCUGCUCCUGUGCUUCACCUACAUCUGCCUG |
| | CACUUCUCUGCUCUUCAGGUAUCACAUCGGUAUCCUCGAAUUCAAAGUAUCAAAGUACAAUUUACCGA |
| | AUAUAAGAAGGAGAAAGGUUUCAUCCUCACUUCCCAAAAGGAGGAUGAAAUCAUGAAGGUGCAGAACA |
| | ACUCAGUCAUCAUCAACUGUGAUGGGUUUUAUCUCAUCUCCCUGAAGGGCUACUUCUCCCAGGAAGUC |
| | AACAUUAGCCUUCAUUACCAGAAGGAUGAGGAGCCCCUCUUCCAACUGAAGAAGGUCAGGUCUGUCAA |
| | CUCCUUGAUGGUGGCCUCUCUGACUUACAAAGACAAAGUCUACUUGAAUGUGACCACUGACAAUACCU |
| | CCCUGGAUGACUUCCAUGUGAAUGGCGGAGAACUGAUUCUUAUCCAUCAAAAUCCUGGUGAAUUCUGU |
| | GUCCUUUGAGGGGCUGAUGGCAAUAUCUAAAACCAGGCACCAGCAUGAACACCAAGCUGGGGGUGGAC |
| | AGGGCAUGGAUUCUUCAUUGCAAGUGAAGGAGCCUCCCAGCUCAGCCACGUGGGAUGUGACAAGAAGC |
| | AGAUCCUGGCCCUCCCGCCCCCACCCCUCAGGGAUAUUUAAAACUUAUUUUAUAUACCAGUUAAUCUU |
| | AUUUAUCCUUAUAUUUUCUAAAUUGCCUAGCCGUCACACCCCAAGAUUGCCUUGAGCCUACUAGGCAC |
| | CUUUGUGAGAAAGAAAAAAUAGAUGCCUCUUCUUCAAGAUGCAUUGUUUCUAUUGGUCAGGCAAUUGU |
| | CAUAAUAAACUUAUGUCAUUGAAAACGGUACCUGACUACCAUUUGCUGGAAAUUUGACAUGUGUGUGG |
| | CAUUAUCAAAAUGAAGAGGAGCAAGGAGUGAAGGAGUGGGGUUAUGAAUCUGCCAAAGGUGGUAUGAA |
| | CCAACCCCUGGAAGCCAAAGCGGCCUCUCCAAGGUUAAAUUGAUUGCAGUUUGCAUAUUGCCUAAAUU |
| | UAAACUUUCUCAUUUGGUGGGGGUUCAAAAGAAGAAUCAGCUUGUGAAAAAUCAGGACUUGAAGAGAG |
| | CCGUCUAAGAAAUACCACGUGCUUUUUUUCUUUACCAUUUUGCUUUCCCAGCCUCCAAACAUAGUUAA |
| | UAGAAAUUUCCCUUCAAAGAACUGUCUGGGGAUGUGAUGCUUUGAAAAAUCUAAUCAGUGACUUAAGA |
| | GAGAUUUUCUUGUAUACAGGGAGAGUGAGAUAACUUAUUGUGAAGGGUUAGCUUUACUGUACAGGAUA |
| | GCAGGGAACUGGACAUCUCAGGGUAAAAGUCAGUACGGAUUUUAAUAGCCUGGGGAGGAAAACACAUU |
| | CUUUGCCACAGACAGGCAAAGCAACACAUGCUCAUCCUCCUGCCUAUGCUGAGAUACGCACUCAGCUC |
| | CAUGUCUUGUACACACAGAAACAUUGCUGGUUUCAAGAAAUGAGGUGAUCCUAUUAUCAAAUUCAAUC |
| | UGAUGUCAAAUAGCACUAAGAAGUUAUUGUGCCUUAUGAAAAAUAAUGAUCUCUGUCUAGAAAUACCA |
| | UAGACCAUAUAUAGUCUCACAUUGAUAAUUGAAACUAGAAGGGUCUAUAAUCAGCCUAUGCCAGGGCU |
| | UCAAUGGAAUAGUAUCCCCUUAUGUUUAGUUGAAAUGUCCCCUUAACUUGAUAUAAUGUGUUAUGCUU |
| | AUGGCGCUGUGGACAAUCUGAUUUUUCAUGUCAACUUUCCAGAUGAUUUGUAACUUCUCUGUGCCAAA |
| | CCUUUUAUAAACAUAAAUUUUUGAGAUAUGUAUUUUAAAAUUGUAGCACAUGUUUCCCUGACAUUUUC |
| | AAUAGAGGAUACAACAUCACAGAAUCUUUCUGGAUGAUUCUGUGUUAUCAAGGAAUUGUACUGUGCUA |
| | CAAUUAUCUCUAGAAUCUCCAGAAAGGUGGAGGGCUGUUCGCCCUUACACUAAAUGGUCUCAGUUGGA |
| | UUUUUUUUUCCUGUUUUCUAUUUCCUCUUAAGUACACCUUCAACUAUAUUCCCAUCCCUCUAUUUUAA |
| | UCUGUUAUGAAGGAAGGUAAAUAAAAAUGCUAAAUAGAAGAAAUUGUAGGUAAGGUAAGAGGAAUCAA |
| | GUUCUGAGUGGCUGCCAAGGCACUCACAGAAUCAUAAUCAUGGCUAAAUAUUUAUGGAGGGCCUACUG |
| | UGGACCAGGCACUGGGCUAAAUACUUACAUUUACAAGAAUCAUUCUGAGACAGAUAUUCAAUGAUAUC |
| | UGGCUUCACUACUCAGAAGAUUGUGUGUGUGUUUGUGUGUGUGUGUGUGUGUGUAUUUCACUUUUUGU |
| | UAUUGACCAUGUUCUGCAAAAUUGCAGUUACUCAGUGAGUGAUAUCCGAAAAAGUAAACGUUUAUGAC |
| | UAUAGGUAAUAUUUAAGAAAAUGCAUGGUUCAUUUUUAAGUUUGGAAUUUUUAUCUAUAUUUCUCACA |
| | GAUGUGCAGUGCACAUGCAGGCCUAAGUAUAUGUUGUGUGUGUUGUUUGUCUUUGAUGUCAUGGUCCC |
| | CUCUCUUAGGUGCUCACUCGCUUUGGGUGCACCUGGCCUGCUCUUCCCAUGUUGGCCUCUGCAACCAC |
| | ACAGGGAUAUUUCUGCUAUGCACCAGCCUCACUCCACCUUCCUUCCAUCAAAAAUAUGUGUGUGUGUC |
| | UCAGUCCCUGUAAGUCAUGUCCUUCACAGGGAGAAUUAACCCUUCGAUAUACAUGGCAGAGUUUUGUG |
| | GGAAAAGAAUUGAAUGAAAAGUCAGGAGAUCAGAAUUUUAAAUUUGACUUAGCCACUAACUAGCCAUG |
| | UAACCUUGGGAAAGUCAUUUCCCAUUUCUGGGUCUUGCUUUUCUUUCUGUUAAAUGAGAGGAAUGUUA |
| | AAUAUCUAACAGUUUAGAAUCUUAUGCUUACAGUGUUAUCUGUGAAUGCACAUAUUAAAUGUCUAUGU |
| | UCUUGUUGCUAUGAGUCAAGGAGUGUAACCUUCUCCUUUACUAUGUUGAAUGUAUUUUUUUCUGGACA |
| | AGCUUACAUCUUCCUCAGCCAUCUUUGUGAGUCCUUCAAGAGCAGUUAUCAAUUGUUAGUUAGAUAUU |
| | UUCUAUUUAGAGAAUGCUUAAGGGAUUCCAAUCCCGAUCCAAAUCAUAAUUUGUUCUUAAGUAUACUG |
| | GGCAGGUCCCCUAUUUUAAGUCAUAAUUUUGUAUUUAGUGCUUUCCUGGCUCUCAGAGAGUAUUAAUA |
| | UUGAUAUUAAUAAUAUAGUUAAUAGUAAUAUUGCUAUUUACAUGGAAACAAAUAAAAGAUCUCAGAAU |
| | UCACUAAAAAAAAAAA |
| | OX40L-TNFSF4, transcript variant 1, mRNA NM_003326 |
| 7 | GGGAAAUAAGAGAGAAAAGAAGAGUAAGAAGAAAUAUAAGAGCCACCAUGGAAAGGGUCCAACCCCUG |
| | GAAGAGAAUGUGGGAAAUGCAGCCAGGCCAAGAUUCGAGAGGAACAAGCUAUUGCUGGUGGCCUCUGU |
| | AAUUCAGGGACUGGGGCUGCUCCUGUGCUUCACCUACAUCUGCCUGCACUUCUCUGCUCUUCAGGUAU |
| | CACAUCGGUAUCCUCGAAUUCAAAGUAUCAAAGUACAAUUUACCGAAUAUAAGAAGGAGAAAGGUUUC |
| | AUCCUCACUUCCCAAAAGGAGGAUGAAAUCAUGAAGGUGCAGAACAACUCAGUCAUCAUCAACUGUGA |
| | UGGGUUUUAUCUCAUCUCCCUGAAGGGCUACUUCUCCCAGGAAGUCAACAUUAGCCUUCAUUACCAGA |
| | AGGAUGAGGAGCCCCUCUUCCAACUGAAGAAGGUCAGGUCUGUCAACUCCUUGAUGGUGGCCUCUCUG |
| | ACUUACAAAGACAAAGUCUACUUGAAUGUGACCACUGACAAUACCUCCCUGGAUGACUUCCAUGUGAA |
| | UGGCGGAGAACUGAUUCUUAUCCAUCAAAAUCCUGGUGAAUUCUGUGUCCUUUGAUAAUAGGCUGGAG |
| | CCUCGGUGGCCAUGCUUCUUGCCCCUUGGGCCUCCCCCCAGCCCCUCCUCCCCUUCCUGCACCCGUAC |
| | CCCCCAAACACCAUUGUCACACUCCAGUGGUCUUUGAAUAAAGUCUGAGUGGGCGGC |
| | mRNA sequence: Human OX40L with 5′-UTR, 3′-UTR, and miR-122 binding |
| | site |
| 8 | AUGGAGCGGGUGCAGCCCCUGGAGGAGAACGUGGGCAACGCCGCUCGGCCACGGUUCGAGCGGAACAA |
| | GCUGCUGCUGGUGGCUAGCGUGAUCCAGGGCCUGGGCCUGCUGCUGUGCUUCACCUACAUCUGCCUGC |
| | ACUUCAGCGCCCUGCAGGUGAGCCACCGGUAUCCCCGGAUCCAGAGCAUCAAGGUGCAGUUCACCGAG |
| | UACAAGAAGGAGAAGGGCUUCAUCCUGACCAGCCAGAAGGAGGACGAGAUCAUGAAGGUGCAGAACAA |
| | CAGCGUGAUCAUCAACUGCGACGGCUUCUACCUGAUCAGCCUGAAGGGCUACUUCAGCCAGGAGGUGA |
| | ACAUCAGCCUGCACUACCAGAAGGACGAGGAGCCCCUGUUCCAGCUGAAGAAGGUGCGGAGCGUGAAC |
| | AGCCUGAUGGUGGCCAGCCUGACCUACAAGGACAAGGUGUACCUGAACGUGACCACCGACAACACCAG |
| | CCUGGACGACUUCCACGUGAACGGCGGCGAGCUGAUCCUGAUCCACCAGAACCCCGGCGAGUUCUGCG |
| | UGCUG |
| | mRNA open reading frame sequence 1 for Human OX40L |
| 9 | AUGGAAAGGGUCCAACCCCUCGAAGAGAACGUGGGAAACGCAGCCAGGCCAAGAUUCGAGAGGAACAA |
| | GCUAUUGCUCGUGGCCUCAGUAAUUCAGGGACUCGGGUUACUCCUUUGCUUCACCUACAUCUGCUUGC |
| | ACUUCAGUGCUCUGCAGGUAUCACAUCGGUAUCCUCGAAUUCAAAGUAUCAAAGUACAAUUUACCGAA |
| | UAUAAGAAGGAGAAAGGUUUCAUCCUCACUUCCCAGAAGGAGGAUGAAAUCAUGAAGGUGCAGAACAA |
| | CUCAGUCAUCAUCAACUGUGAUGGGUUUUAUCUCAUCUCCCUGAAGGGCUACUUCUCCCAGGAAGUCA |
| | ACAUUAGCCUUCAUUACCAGAAGGAUGAGGAGCCCCUCUUCCAACUGAAGAAGGUCAGGUCUGUCAAC |
| | UCCUUGAUGGUAGCCUCUCUGACUUACAAAGACAAAGUCUACUUGAAUGUGACCACUGACAAUACCUC |
| | CCUGGAUGACUUCCAUGUGAAUGGCGGAGAACUGAUUCUUAUCCAUCAGAAUCCUGGUGAAUUCUGUG |
| | UCCUU |
| | mRNA open reading frame sequence 2 for Human OX40L |
| 10 | AUGGAGCGGGUGCAGCCCCUGGAGGAGAACGUGGGCAACGCCGCUCGGCCACGGUUCGAGCGGAACAA |
| | GCUGCUGCUGGUGGCUAGCGUGAUCCAGGGCCUGGGCCUGCUGCUGUGCUUCACCUACAUCUGCCUGC |
| | ACUUCAGCGCCCUGCAGGUGAGCCACCGGUAUCCCCGGAUCCAGAGCAUCAAGGUGCAGUUCACCGAG |
| | UACAAGAAGGAGAAGGGCUUCAUCCUGACCAGCCAGAAGGAGGACGAGAUCAUGAAGGUGCAGAACAA |
| | CAGCGUGAUCAUCAACUGCGACGGCUUCUACCUGAUCAGCCUGAAGGGCUACUUCAGCCAGGAGGUGA |
| | ACAUCAGCCUGCACUACCAGAAGGACGAGGAGCCCCUGUUCCAGCUGAAGAAGGUGCGGAGCGUGAAC |
| | AGCCUGAUGGUGGCCAGCCUGACCUACAAGGACAAGGUGUACCUGAACGUGACCACCGACAACACCAG |
| | CCUGGACGACUUCCACGUGAACGGCGGCGAGCUGAUCCUGAUCCACCAGAACCCCGGCGAGUUCUGCG |
| | UGCUG |
| | mRNA open reading frame sequence 3 for Human OX40L |
| 11 | AGGAAAUAAGAGAGAAAAGAAGAGUAAGAAGAAAUAUAAGACCCCGGCGCCGCCACC |
| | 5′ UTR |
| 12 | CCUUAGCAGAGCUGUGGAGUGUGACAAUGGUGUUUGUGUCUAAACUAUCAAACGCCAUUAUCACACUA |
| | AAUAGCUACUGCUAGGC |
| | (miR-122) |
| 13 | AACGCCAUUAUCACACUAAAUA |
| | (miR-122-3p) |
| 14 | UAUUUAGUGUGAUAAUGGCGUU (miR-122-3p binding site) |
| 15 | UCAAGCUUUUGGACCCUCGUACAGAAGCUAAUACGACUCACUAUAGGGAAAUAAGAGAGAAAAGAAGA |
| | GUAAGAAGAAAUAUAAGAGCCACC |
| | (5′ UTR) |
| 16 | GGGAAAUAAGAGAGAAAAGAAGAGUAAGAAGAAAUAUAAGAGCCACC |
| | (5′ UTR) |
| 17 | UGAUAAUAGGCUGGAGCCUCGGUGGCCAUGCUUCUUGCCCCUUGGGCCUCCCCCCAGCCCCUCCUCCC |
| | CUUCCUGCACCCGUACCCCCCAAACACCAUUGUCACACUCCAGUGGUCUUUGAAUAAAGUCUGAGUGG |
| | GCGGC (3′ UTR) |
| 18 | UGAUAAUAGGCUGGAGCCUCGGUGGCCAUGCUUCUUGCCCCUUGGGCCCAAACACCAUUGUCACACUC |
| | CAUCCCCCCAGCCCCUCCUCCCCUUCCUCCAUAAAGUAGGAAACACUACAUGCACCCGUACCCCCGUG |
| | GUCUUUGAAUAAAGUCUGAGUGGGCGGC |
| | (3′ UTR with mi-122 and mi-142.3p sites) |
| 19 | UGGAGUGUGACAAUGGUGUUUG |
| | (miR-122-5p) |
| 20 | CAAACACCAUUGUCACACUCCA (miR-122-5p binding site) |
| 21 | GCCA/GCC |
| | Kozak Consensus |
| 22 | UGAUAAUAGGCUGGAGCCUCGGUGGCCUAGCUUCUUGCCCCUUGGGCCUCCCCCCAGCCCCUCCUCCC |
| | CUUCCUGCACCCGUACCCCCCAAACACCAUUGUCACACUCCAGUGGUCUUUGAAUAAAGUCUGAGUGG |
| | GCGGC |
| | 3′ UTR with miR-122 |
| 23 | CCGCCGCCGCCG |
| | [CCG]₄ |
| 24 | CCGCCGCCGCCGCCG |
| | [CCG]₅ |
| 25 | CCCCGGCGCC |
| | V1 GC-rich RNA element |
| 26 | CCCCGGC |
| | V2 GC-rich RNA element |
| 27 | GCCGCC |
| | EK GC-rich RNA element |
| 28 | GGGAAATAAGAGAGAAAAGAAGAGTAAGAAGAAATATAAGA |
| | 5′ UTR |
| 29 | GGGAAATAAGAGAGAAAAGAAGAGTAAGAAGAAATATAAGACCCCGGCGCCGCCACC |
| | V1-5′ UTR |
| 30 | GGGAAATAAGAGAGAAAAGAAGAGTAAGAAGAAATATAAGACCCCGGCGCCACC |
| | V2-5′ UTR |
| 31 | GGGAAATAAGAGAGAAAAGAAGAGTAAGAAGAAATATAAGAGCCACC |
| | Standard UTR |

What is claimed is:

1. A method for treating ovarian cancer, or a solid tumor, lymphoma or epithelial origin cancer, in a human patient by inducing or enhancing an anti-tumor immune response, comprising administering to the patient by intratumoral injection an effective amount of a pharmaceutical composition comprising: a lipid nanoparticle (LNP) comprising a messenger RNA (mRNA) encoding a human OX40L polypeptide; and a pharmaceutically acceptable carrier, thereby treating ovarian cancer, or a solid tumor, lymphoma or epithelial origin cancer, in the patient by inducing or enhancing an anti-tumor immune response.

2. The method of claim 1, wherein treatment results in a reduction in tumor size or inhibition in tumor growth in the injected tumor in the patient.

3. The method of claim 1, wherein treatment results in a reduction in size or inhibition of growth in an uninjected tumor in the patient.

4. The method of claim 1, wherein the anti-tumor immune response in the patient comprises at least one of an inflammatory response, T cell activation, T cell proliferation, and T cell expansion.

5. The method of claim 4, wherein the anti-tumor immune response results in a reduction in size or inhibition of growth of the injected tumor.

6. The method of claim 4, wherein the anti-tumor immune response results in a reduction in size or inhibition of growth of an uninjected tumor through an abscopal effect in the patient.

7. The method of claim 1, wherein the patient is administered a dose of mRNA selected from 1.0-8.0 mg, 1.0-6.0 mg, 1.0-4.0 mg, and 1.0-2.0 mg of mRNA.

8. The method of claim 1, wherein the mRNA is administered in a dosing regimen selected from 7 to 28 days, 7 to 21 days, 7 to 14 days, 28 days, 21 days, 14 days and 7 days.

9. The method of claim 1, wherein the mRNA is administered every 2 weeks in a 28-day cycle.

10. The method of claim 1, wherein the mRNA is administered at a dose of 8.0 mg.

11. A method for treating ovarian cancer, or a solid tumor, lymphoma or epithelial origin cancer, in a human patient by inducing or enhancing an anti-tumor immune response, comprising administering to the patient by intratumoral injection an effective amount of a pharmaceutical composition comprising: an LNP comprising an mRNA encoding a human OX40L polypeptide; and a pharmaceutically acceptable carrier, wherein the patient is administered a dose of 1.0-8.0 mg of mRNA in a dosing regimen from 7 to 21 days, thereby treating ovarian cancer, or a solid tumor, lymphoma or epithelial origin cancer, in the patient by inducing or enhancing an anti-tumor immune response.

12. The method of claim 11, wherein the patient is administered a dose of 1.0-8.0 mg mRNA.

13. The method of any one of claim 11, wherein the dose is administered every 14 days.

14. The method of any one of claim 11, wherein the mRNA is administered every 2 weeks in a 28-day cycle.

15. The method of claim 11, wherein the mRNA is administered every 2 weeks for 1-6 months.

16. The method of claim 11, wherein the mRNA is administered on day 1 and day 15 (±2 days) of a 28-day cycle until the tumor lesion resolves.

17. The method of claim 11, wherein treatment results in a reduction in tumor size or inhibition in tumor growth in the injected tumor in the patient.

18. The method of claim 11, wherein the mRNA is administered by a single injection.

19. The method of claim 11, wherein the mRNA is administered by multiple injections into one or more different sites within the same tumor lesion or divided across several tumor lesions.

20. The method of claim 11, wherein the human OX40L polypeptide comprises the amino acid sequence set forth in SEQ ID NO: 1.

21. The method of claim 11, wherein the mRNA comprises an open reading frame comprising a nucleotide sequence at least 90% identical to the nucleotide sequence set forth in SEQ ID NO: 4.

22. The method of claim 11, wherein the mRNA comprises a 3′ untranslated region (UTR) comprising at least one microRNA-122 (miR-122) binding site.

23. The method of claim 11, wherein the mRNA comprises a nucleotide sequence at least 90% identical to the nucleotide sequence set forth in SEQ ID NO: 5.

24. The method of claim 11, wherein the mRNA is chemically modified.

25. The method of claim 11, wherein the LNP comprises a compound having the formula:

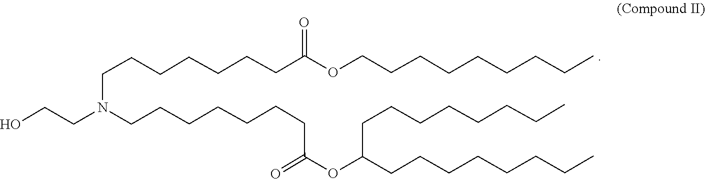 [SMILES: CCCCCCCCCOC(=O)CCCCCCCN(CCO)CCCCCCCC(=O)OC(CCCCCCCC)CCCCCCCC]

26. The method of claim 25, wherein the LNP further comprising a phospholipid, a structural lipid, and a PEG lipid.

27. The method of claim 26, wherein the LNP comprises a molar ratio of about 20-60% ionizable amino lipid, about 5-25% phospholipid, about 25-55% structural lipid, and about 0.5-1.5% PEG lipid.

28. The method of claim 26, wherein the LNP comprises a molar ratio of about 50% ionizable amino lipid, about 10% phospholipid, about 38.5% structural lipid, and about 1.5% PEG lipid.

29. The method of claim 11, further comprising administering an effective amount of a PD-1 antagonist, a PD-L1 antagonist or a CTLA-4 antagonist.


SEQUENCE LISTING (31 sequences)

SEQ ID NO: 1 - protein, 183 aa, Homo sapiens
  misc_feature (1)..(183): OX40L (TNSFR4) - Tumor necrosis factor ligand superfamily member 4 isoform 1
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - protein, 133 aa, Homo sapiens
  misc_feature (1)..(133): OX40L (TNSFR4) - TNFSF4 isoform 2
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - protein, 198 aa, Mus musculus
  misc_feature (1)..(198): OX40L (TNSFR4) - TNFSF4
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 4 - RNA, 549 nt, Homo sapiens
  misc_feature (1)..(549): Human OX40L mRNA (ORF)
auggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuu

SEQ ID NO: 5 - RNA, 841 nt, Homo sapiens
  misc_feature (1)..(841): A,C G and U = AMP, CMP, GMP and N1-psi-UMP, respectively
  misc_feature (1)..(1): 57MeGpppG2Ome (Me = methyl; p = inorganic phosphate)
  misc_feature (1)..(841): Full-length mRNA Nucleotide sequence (5 UTR, ORF, 3 UTR, polyA tail) of human OX40L
  misc_feature (841)..(841): OH3
ggaaauaagagagaaaagaagaguaagaagaaauauaagagccaccauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugauaauaggcuggagccucgguggccaugcuucuugccccuugggccuccccccagccccuccuccccuuccugcacccguaccccccaaacaccauugucacacuccaguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 6 - RNA, 3484 nt, Homo sapiens
  misc_feature (1)..(3484): OX40L - TNFSF4, transcript variant 1, mRNA
ggcccugggaccuuugccuauuuucugauugauaggcuuuguuuugucuuuaccuccuucuuucuggggaaaacuucaguuuuaucgcacguuccccuuuuccauaucuucaucuucccucuacccagauugugaagauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugaggggcugauggcaauaucuaaaaccaggcaccagcaugaacaccaagcuggggguggacagggcauggauucuucauugcaagugaaggagccucccagcucagccacgugggaugugacaagaagcagauccuggcccucccgcccccaccccucagggauauuuaaaacuuauuuuauauaccaguuaaucuuauuuauccuuauauuuucuaaauugccuagccgucacaccccaagauugccuugagccuacuaggcaccuuugugagaaagaaaaaauagaugccucuucuucaagaugcauuguuucuauuggucaggcaauugucauaauaaacuuaugucauugaaaacgguaccugacuaccauuugcuggaaauuugacauguguguggcauuaucaaaaugaagaggagcaaggagugaaggagugggguuaugaaucugccaaaggugguaugaaccaaccccuggaagccaaagcggccucuccaagguuaaauugauugcaguuugcauauugccuaaauuuaaacuuucucauuugguggggguucaaaagaagaaucagcuugugaaaaaucaggacuugaagagagccgucuaagaaauaccacgugcuuuuuuucuuuaccauuuugcuuucccagccuccaaacauaguuaauagaaauuucccuucaaagaacugucuggggaugugaugcuuugaaaaaucuaaucagugacuuaagagagauuuucuuguauacagggagagugagauaacuuauugugaaggguuagcuuuacuguacaggauagcagggaacuggacaucucaggguaaaagucaguacggauuuuaauagccuggggaggaaaacacauucuuugccacagacaggcaaagcaacacaugcucauccuccugccuaugcugagauacgcacucagcuccaugucuuguacacacagaaacauugcugguuucaagaaaugaggugauccuauuaucaaauucaaucugaugucaaauagcacuaagaaguuauugugccuuaugaaaaauaaugaucucugucuagaaauaccauagaccauauauagucucacauugauaauugaaacuagaagggucuauaaucagccuaugccagggcuucaauggaauaguauccccuuauguuuaguugaaauguccccuuaacuugauauaauguguuaugcuuauggcgcuguggacaaucugauuuuucaugucaacuuuccagaugauuuguaacuucucugugccaaaccuuuuauaaacauaaauuuuugagauauguauuuuaaaauuguagcacauguuucccugacauuuucaauagaggauacaacaucacagaaucuuucuggaugauucuguguuaucaaggaauuguacugugcuacaauuaucucuagaaucuccagaaagguggagggcuguucgcccuuacacuaaauggucucaguuggauuuuuuuuuccuguuuucuauuuccucuuaaguacaccuucaacuauauucccaucccucuauuuuaaucuguuaugaaggaagguaaauaaaaaugcuaaauagaagaaauuguagguaagguaagaggaaucaaguucugaguggcugccaaggcacucacagaaucauaaucauggcuaaauauuuauggagggccuacuguggaccaggcacugggcuaaauacuuacauuuacaagaaucauucugagacagauauucaaugauaucuggcuucacuacucagaagauuguguguguguuuguguguguguguguguguguauuucacuuuuuguuauugaccauguucugcaaaauugcaguuacucagugagugauauccgaaaaaguaaacguuuaugacuauagguaauauuuaagaaaaugcaugguucauuuuuaaguuuggaauuuuuaucuauauuucucacagaugugcagugcacaugcaggccuaaguauauguuguguguguuguuugucuuugaugucaugguccccucucuuaggugcucacucgcuuugggugcaccuggccugcucuucccauguuggccucugcaaccacacagggauauuucugcuaugcaccagccucacuccaccuuccuuccaucaaaaauaugugugugugucucagucccuguaagucauguccuucacagggagaauuaacccuucgauauacauggcagaguuuugugggaaaagaauugaaugaaaagucaggagaucagaauuuuaaauuugacuuagccacuaacuagccauguaaccuugggaaagucauuucccauuucugggucuugcuuuucuuucuguuaaaugagaggaauguuaaauaucuaacaguuuagaaucuuaugcuuacaguguuaucugugaaugcacauauuaaaugucuauguucuuguugcuaugagucaaggaguguaaccuucuccuuuacuauguugaauguauuuuuuucuggacaagcuuacaucuuccucagccaucuuugugaguccuucaagagcaguuaucaauuguuaguuagauauuuucuauuuagagaaugcuuaagggauuccaaucccgauccaaaucauaauuuguucuuaaguauacugggcagguccccuauuuuaagucauaauuuuguauuuagugcuuuccuggcucucagagaguauuaauauugauauuaauaauauaguuaauaguaauauugcuauuuacauggaaacaaauaaaagaucucagaauucacuaaaaaaaaaaa

SEQ ID NO: 7 - RNA, 737 nt, Homo sapiens
  misc_feature (1)..(737): mRNA sequence Human OX40L with 5′-UTR, 3′-UTR, and miR-122 binding site
gggaaauaagagagaaaagaagaguaagaagaaauauaagagccaccauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 8 - RNA, 549 nt, Homo sapiens
  misc_feature (1)..(549): mRNA open reading frame sequence 1 for Human OX40L
auggagcgggugcagccccuggaggagaacgugggcaacgccgcucggccacgguucgagcggaacaagcugcugcugguggcuagcgugauccagggccugggccugcugcugugcuucaccuacaucugccugcacuucagcgcccugcaggugagccaccgguauccccggauccagagcaucaaggugcaguucaccgaguacaagaaggagaagggcuucauccugaccagccagaaggaggacgagaucaugaaggugcagaacaacagcgugaucaucaacugcgacggcuucuaccugaucagccugaagggcuacuucagccaggaggugaacaucagccugcacuaccagaaggacgaggagccccuguuccagcugaagaaggugcggagcgugaacagccugaugguggccagccugaccuacaaggacaagguguaccugaacgugaccaccgacaacaccagccuggacgacuuccacgugaacggcggcgagcugauccugauccaccagaaccccggcgaguucugcgugcug

SEQ ID NO: 9 - RNA, 549 nt, Homo sapiens
  misc_feature (1)..(549): mRNA open reading frame sequence 2 for Human OX40L
auggaaaggguccaaccccucgaagagaacgugggaaacgcagccaggccaagauucgagaggaacaagcuauugcucguggccucaguaauucagggacucggguuacuccuuugcuucaccuacaucugcuugcacuucagugcucugcagguaucacaucgguauccucgaauucaaaguaucaaaguacaauuuaccgaauauaagaaggagaaagguuucauccucacuucccagaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguagccucucugacuuacaaagacaaagucuacuugaaugugaccacugacaauaccucccuggaugacuuccaugugaauggcggagaacugauucuuauccaucagaauccuggugaauucuguguccuu

SEQ ID NO: 10 - RNA, 549 nt, Homo sapiens; identical to SEQ ID NO: 8
  misc_feature (1)..(549): mRNA open reading frame sequence 3 for Human OX40L

SEQ ID NO: 11 - RNA, 57 nt, Artificial Sequence
  Synthetic 5UTR
aggaaauaagagagaaaagaagaguaagaagaaauauaagaccccggcgccgccacc

SEQ ID NO: 12 - RNA, 85 nt, Artificial Sequence
  Synthetic miR-122
ccuuagcagagcuguggagugugacaaugguguuugugucuaaacuaucaaacgccauuaucacacuaaauagcuacugcuaggc

SEQ ID NO: 13 - RNA, 22 nt, Artificial Sequence
  Synthetic miR-122-3p
aacgccauuaucacacuaaaua

SEQ ID NO: 14 - RNA, 22 nt, Artificial Sequence
  Synthetic miR-122-3p binding site
uauuuagugugauaauggcguu

SEQ ID NO: 15 - RNA, 92 nt, Artificial Sequence
  Synthetic 5 UTR
ucaagcuuuuggacccucguacagaagcuaauacgacucacuauagggaaauaagagagaaaagaagaguaagaagaaauauaagagccacc

SEQ ID NO: 16 - RNA, 47 nt, Artificial Sequence
  Synthetic 5 UTR
gggaaauaagagagaaaagaagaguaagaagaaauauaagagccacc

SEQ ID NO: 17 - RNA, 141 nt, Artificial Sequence
  Synthetic 3 UTR
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 18 - RNA, 164 nt, Artificial Sequence
  Synthetic 3 UTR with mi-122 and mi-142.3p sites
ugauaauaggcuggagccucgguggccaugcuucuugccccuugggcccaaacaccauugucacacuccauccccccagccccuccuccccuuccuccauaaaguaggaaacacuacaugcacccguacccccguggucuuugaauaaagucugagugggcggc

SEQ ID NO: 19 - RNA, 22 nt, Artificial Sequence
  Synthetic miR-122-5p
uggagugugacaaugguguuug

SEQ ID NO: 20 - RNA, 22 nt, Artificial Sequence
  Synthetic miR-122-5p binding site
caaacaccauugucacacucca

SEQ ID NO: 21 - RNA, 6 nt, Artificial Sequence
  Synthetic Kozak Consensus
gccrcc

SEQ ID NO: 22 - RNA, 141 nt, Artificial Sequence
  Synthetic 3UTR with miR-122
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 23 - RNA, 12 nt, Artificial Sequence
  Synthetic [CCG]4
ccgccgccgccg

SEQ ID NO: 24 - RNA, 15 nt, Artificial Sequence
  Synthetic [CCG]5
ccgccgccgccgccg

SEQ ID NO: 25 - RNA, 10 nt, Artificial Sequence
  Synthetic V1 GC-rich RNA element
ccccggcgcc

SEQ ID NO: 26 - RNA, 7 nt, Artificial Sequence
  Synthetic V2 GC-rich RNA element
ccccggc

SEQ ID NO: 27 - RNA, 6 nt, Artificial Sequence
  Synthetic EK GC-rich RNA element
gccgcc

SEQ ID NO: 28 - DNA, 41 bp, Artificial Sequence
  Synthetic 5UTR
gggaaataagagagaaaagaagagtaagaagaaatataaga

SEQ ID NO: 29 - DNA, 57 bp, Artificial Sequence
  Synthetic V1-5UTR
gggaaataagagagaaaagaagagtaagaagaaatataagaccccggcgccgccacc

SEQ ID NO: 30 - DNA, 54 bp, Artificial Sequence
  Synthetic V2-5UTR
gggaaataagagagaaaagaagagtaagaagaaatataagaccccggcgccacc

SEQ ID NO: 31 - DNA, 47 bp, Artificial Sequence
  Synthetic Standard UTR
gggaaataagagagaaaagaagagtaagaagaaatataagagccacc